SEQUENCE LISTING (73 sequences)

SEQ ID NO: 1 - DNA, 552 bp, Mycobacterium smegmatis
  source 1..552: mol_type = genomic DNA
ggcctggataacgaacttagcctggtggacggccaagatcgcacgctgacggtgcaacaatgggataccttcctgaatggtgtgtttccgctggatcgtaaccgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcgatgacggtgatattaccgcaccgccgtttggcctgaacagcgtgattacgccgaacctgtttccgggtgttagcatctctgccgatctgggcaacggtccgggcattcaagaagtggcaacctttagtgtggacgtttccggcgctgaaggcggtgtcgcggtgtctaatgcccacggtaccgttacgggcgcggccggcggtgtcctgctgcgtccgttcgcgcgcctgattgcgagcaccggcgactctgttacgacctatggcgaaccgtggaatatgaac

SEQ ID NO: 2 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMN

SEQ ID NO: 3 - DNA, 885 bp, synthetic construct
  misc_feature 1..885: note = Synthetic polynucleotide
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = Synthetic polypeptide
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - protein, 439 aa, unidentified
  source 1..439: note = Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - protein, 678 aa, Rhodothermus marinus
MEELSNEQQRVLDHVLAWLERNDAPPIFILTGSAGTGKTLLIRHLVRALQDRRIHYALAAPTGRAARILSERTGDHARTLHSLIYIFDRYQLVEEADRQTDEPLSLQLHFALRSAEHDARLIIVDEASMVSDTAGEEELYRFGSGRLLNDLLTFARLIPKRDRPPTTRLLFVGDPAQLPPVGQSVSPALSAQYLRDTFGLSAETAHLRSVYRQRKGHPILETATALRNALEKGHYHTFRLPEQPPDLRPVGLEEAIETTATDFRRQNPSVLLCRTNALARKLNAAVRARLWGREGLPPQPGDLLLVNRNAPLHGLFNGDLVLVETVGPLEHRRVGRRGRPPVDLYFRDVELLYPHEKPRNRIRCKLLENLLESPDGQLSPDIIQALLIDFYRRHPSLKHGSSEFRLMLANDAYFNALHVRYGYAMTVHKAQGGEWKRATVVFNDWRHFRHAEFFRWAYTAITRAREELLTIGAPSFEALSDMRWQPAPSVPAPEQAAENATRFPLKALETYHQRLSEALTAAGIETTGVELLQYAVRYHLARADRTTRIQYYYRGDGQISRIVTLGGADDPELTQQAYALFERILSEPPADSGELPENPLLREFLERAHLRLEGSGIRIVHWKEMPYALRLYFSADGENVTIDFYYNRRGVWTHAQEVGRSSSGALFARIQSLLQADS

SEQ ID NO: 10 - protein, 496 aa, unidentified
  source 1..496: note = Cyanothece ATCC51142
MSQSVVVPDELGEIITAVIEFYQDAVDKIEPKIVFLELRKNVVDWVSRTQLKIEEKEIQATGLTRQQQTAYKEMINFIENSSEQYFRLSGYAGTGKSFLMAKVIEWLKQEDYKYSVAAPTNKAAKNLTQIARSQGIKIEATTVAKLLKLQPTIDVDTGQQSFEFNSEKELELKDYDVIIIDEYSMLNKDNFRDLQQAVKGGESKFIFVGDSSQLPPVKEKEPIVANHPDIRKSANLTQIVRYDGEIVKVAESIRRNPRWNHQTYPFETVADGTIIKLNTEDWLQQALSHFEKEDWLSNPDYVRMITWRNKTADKYNQAIREALYGENVEQLVVGDRLIAKKPVFRSLPGGKKKEKKIILNNSEECKVIETPKINYNEKYKWEFYQVKVRTDEGGMIELRILTSESEEKRQKKLKELAKRAREEENYSEKKKQWAIYYELDELFDNMAYAYALTCHKAQGSSIDNVFLLVSDMHYCRDKTKMIYTGLTRAKKCCYVG

SEQ ID NO: 11 - protein, 421 aa, Salinibacter ruber
MSTFADAPFTEDQEEAYDHVYDRLAQGERFTGLRGYAGTGKTYLVSRLVEQLLDEDCTVTVCAPTHKAVQVLSDELGDAPVQMQTLHSFLGLRLQPKQDGEYELVAEEERNFAEGVVIVDEASMIGREEWSHIQDAPFWVQWLFVGDPAQLPPVNEDPSPALDVPGPTLETIHRQAADNPILELATKIRTGADGRFGSTFEDGKGVAVTRNREEFLDSILRAFDADAFAEDATHARVLAYRNKTVRRYNREIRAERYGADADRFVEGEWLVGTETWYYDGVQRLTNSEEVRVKKAQVETFEADDQSEWTVWELKIRTPGRGLTRTIHVLHEEERERYENALERRRGKAEDDPSKWDRFFELRERFARVDYAYATTVHRAQGSTYDTVFVDHRDLRVCRGEERGALLYVAVTRPSRRLALLV

SEQ ID NO: 12 - protein, 500 aa, Sulfurimonas gotlandica
  source 1..500: note = GD1
MKILNKETYKLSLHQEEVFTQIVSQLDTKVSSILKSTNIEDYLLSLTGPAGTGKTFLTTQIAKYLVEKRKESEYPMSSDFDFTITAPTHKAVGVLSKLLRENNIQSSCKTIHSFLGIKPFIDYTTGEEKFVVDKTNKRKDRTSILIVDESSMIGNTLYEYILEAIEDKRVNVVLFIGDPYQLLPIENSKNEIYDLPNRFFLSEVVRQAENSYIIRVATKLRERIKNQDFISLQQFFQENMEDEITFFHNKEAFLEDFYKEEEWYKENKILATYKNKDVDAFNKIIRNKFWEQKGNTTPSTLLAGDMIRFKDAYTVGDITIYHNGQELQLGSTEVKYHDSLHIEYWECKSIYALEQQVFRVVNPDSEAVFNQKLQSLATKAKQAKFPDNKKLWKLYYETRNMFANVQYIHASTIHKLQGSTYDVSYIDIFSLVHNHYMSDEEKYRLLYVAITRASKDIKIFMSAFDRTSDEKVIINNQNSETMNTLKQLHDIDIILKDLDL

SEQ ID NO: 13 - protein, 450 aa, unidentified
  source 1..450: note = Vibrio phage henriette 12B8
MADFELTLGQKTVLGEVISTILKPVNLNDTSRFHTMHGPAGSGKTTVLQRIISQIPAYKTIGFCSPTHKSVKVIRRMAREAGISHRVDIRTIHSALGLVMKPVRGDEVLVKEPFAEERIYDVLIIDEAGMLNDELIMYILESQSSKVIFVGDMCQIGPIQSNLPEEDGYTPTSTDDVSKVFTEVEMMSALTEVVRQAEGSPIIQLATEFRLAQDDIYADLPRIVTNTTPDGNGIITMPNGNWVDSAVARFQSDQFKEDPDHCRIVCYTNAMVDLCNDLVRKRLFGADVPEWLEDEILVAQEMGSTWNNADELRIVSIDDHFDQQYEVPCWRMQLESVEDHKLHNALVVKGDYIEDFKFRLNAIAERANTDKNMSGMHWKEFWGMRKKFNTFKNVYAGTAHKSQGSTFDYTYVFTPDFYKFGATMTIKRLLYTAITRSRYTTYFAMNTGAQ

SEQ ID NO: 14 - protein, 421 aa, unidentified
  source 1..421: note = Vibrio phage phi-pp2
MGLTNCQQGAMDAFLESDGHMTISGPAGSGKTFLMKSILEALESKGKNVTMVTPTHQAKNVLHKATGQEVSTIHSLLKIHPDTYEDQKHFTQSGEVEGLDEIDVLVVEEASMVDEELFQITGRTMPRKCRILAVGDKYQLQPVKHDPGVISPFFTKFTTFEMNEVVRQAKDNPLIQVATEVRNGQWLRTNWSKERRQGVLHVPNVNKMLDTYLSKVNSPEDLLDYRILAYTNDCVDTFNGIIREHVYNTSEPFIPGEYLVTQMPVMVSNGKYPVCVIENGEVVKILDVRQKTIDGMLPKVDNEAFDVAVLTVEKEDGNVYEFTVLWDDLQKERFARYLSVAAGTYKSMRGNTKRYWRAFWGLKEQMIETKSLGASTVHKSQGTTVKGVCLYTQDMGYAEPEILQQLVYVGLTRPTDWALYN

SEQ ID NO: 15 - protein, 434 aa, unidentified
  source 1..434: note = Aeromonas phage 65
MSESEITLTPSQNMAVNEVKNGTGHITISGPPGSGKTFLVKYLIKMLGDELGTVLAAPTHQAKIVLTEMSGIEACTIHSLMKIHPETLEDIQIFDQSKLPDLSNIRYLIVEEASMHSKTLFKITMKSIPPTCRIIAIGDKDQIQPEEHAQGELSPYFTDPRFSQIRLTDIMRQSLDNPIIQVATKIREGGWIEPNWNRDTKTGVYKVSGITDLVNSYLRAVKTPEDLTKYRFLAYTNKVVNKVNSIVREHVYKTKLPFIEGEKIVLQEPVMVEHEDDTIETIFTNGEVVTINEIEVFDRTIRIDGSPEFKVNAAKLSVSSDYSGIEHDFCVLYGSESRLEFEYQLSESAGNIKQMGKGGNQRSAWKSFWAAKKMFIETKSLGASTIHKSQGSTVKGVWLALHDIHYADEELKQQLVYVGVTRPTDFCLYFDGTK

SEQ ID NO: 16 - protein, 420 aa, unidentified
  source 1..420: note = Aeromonas phage CC2
MAVDAVQSGTGHITISGPPGSGKTFLVKYIIKMLGDELGTVLAAPTHQAKIVLTEMSGIEACTIHSLMKIHPETLEDIQIFDQSKMPDLSTVRYLIIEEASMHSKALFNITMKSIPPTCRIIAIGDKDQIQPVDHAPGELSPYFTDSRFTQIRMTDIMRQSLDNPIIQVATTIREGGWIYQNWNKEKKSGVYKVKSITDLINSYLRVVKTPEDLTKYRFLAFTNKVVDKVNSIVRKHVYKTDLPFIEGEKLVLQEPVMVEYDDDTIETIFTNGEVVTVDEIEVSDMNIRIDGSPAFSISVAKLKVTSDFSGVTHDIMSVYGEDSKAEFNYQLSEAAAVIKQMQRGQTKAAWASFWDAKKTFTETKSLGACTIHKSQGSTVKGVWLGLHDISYADTDLQQQLVYVGVTRPTDFCLYFDGSK

SEQ ID NO: 17 - protein, 443 aa, unidentified
  source 1..443: note = Cronobacter phage vB CsaM GAP161
MSELTFDDLSDDQKSAHDRVIHNIQNAIHTTITGGPGVGKTTLVKFVFNTLKGLGISGIWLTAPTHQAKNVLAAATGMDATTIHSALKISPVTNEELRVFEQQKGKKAPDLSTCRVFVVEEVSMVDMDLFRIIRRSIPSNAVILGLGDKDQIRPVNADGRVELSPFFDEEIFDVIRMDKIMRQAEGNPIIQVSRAVRDGKMLKPMSVGDLGVFQHANAVDFLRQYFRRVKTPDDLIENRMFAYTNDNVDKLNATIRKHLYKTTEPFILDEVIVMQEPLVQEMRLNGQIFTEIVYNNNEKIRVLEIIPRREVIKAEKCDEKIEIEFYLLKTVSLEEETEAQIQVVVDPVMKDRLGNYLAYVASTYKRIKQQTGYKAPWHSFWAIKNKFQDVKPLPVCTYHKSQGSTYDHAYMYTRDAYAFADYDLCKQLIYVGVTRARYTVDYV

SEQ ID NO: 18 - protein, 442 aa, unidentified
  source 1..442: note = Klebsiella phage KP15
MSELTFDDLSEDQKNAHDRVIKNIRNKIHTTITGGPGVGKTTLVKFVFETLKKLGISGIWLTAPTHQAKNVLSEAVGMDATTIHSALKISPVTNEELRVFEQQKGKKAADLSECRVFVVEEVSMVDKELFRIIKRTIPSCAVILGLGDKDQIRPVNTEGITELSPFFDEEIFDVIRMDKIMRQAEGNPIIQVSRAIRDGKPLMPLMNGELGVMKHENASDFLRRYFSRVKTPDDLNNNRMFAYTNANVDKLNAVIRKHLYKTDQPFIVGEVVVMQEPLVTEGRVNGVSFVEVIYNNNEQIKILEIIPRSDTIKADRCDPVQIDYFLMKTESMFEDTKADIQVIADPVMQERLGDYLNYVAFQYKKMKQETGYKAPWYSFWQIKNKFQTVKALPVCTYHKGQGSTYDHSYMYTRDAYAYADYELCKQLLYVGTTRARFTVDYV

SEQ ID NO: 19 - protein, 438 aa, unidentified
  source 1..438: note = Stenotrophomonas phage IME13
MVTYDDLTVGQKDAIEKALQAMRTKRHITIRGPAGSGKTTMTRFLLERLFQTGQQGIVLTAPTHQAKKELSKHALRKSYTIQSVLKINPSTLEENQIFEQKGTPDFSKTRVLICDEVSFYTRKLFDILMRNVPSHCVVIGIGDKAQIRGVSEDDTHELSPFFTDNRFEQVELTEVKRHQGPIIEVATDIRNGKWIYEKLDDSGNGVKQFHTVKDFLSKYFERTKTPNDLLENRIMAYTNNSVDKLNSVIRKQLYGANAAPFLPDEILVMQEPLMFDIDIGGQTLKEVIFNNGQNVRVINVKPSRKTLKAKGVGEIEVECTMLECESYEEDEDDYRRAWFTVVHDQNTQYAINEFLSIIAEKYRSREVFPNWKDFWAIRNTFVKVRPLGAMTFHKSQGSTFDNAYLFTPCLHQYCRDPDVAQELIYVGNTRARKNVCFV

SEQ ID NO: 20 - protein, 442 aa, unidentified
  source 1..442: note = Acinetobacter phage Ac42
MNFEDLTEGQKNAYTAAIKAIETVPSSSAEKRHLTINGPAGTGKTTLTKFLIAELIRRGERGVYLAAPTHQAKKVLSQHAGMEASTIHSLLKINPTTYEDSTTFEQKDVPDMSECRVLICDEASMYDLKLFQILMSSIPLCCTVIALGDIAQIRPVEPGAFEGQVSPFFTYEKFEQVSLTEVMRSNAPIIDVATSIRTGNWIYENVIDGAGVHNLTSERSVKSFMEKYFSIVKTPEDLFENRLLAFTNKSVDDLNKIVRKKIYNTLEPFIDGEVLVMQEPLIKSYTYEGKKVSEIVFNNGEMVKVLCCSQTSDEISVRGCSTKYMVRYWQLDLQSLDDPDLTGSINVIVDEAEINKLNLVLGKSAEQFKSGAVKAAWADWWKLKRNFHKVKALPCSTIHKSQGTSVDNVFLYTPCIHKADSQLAQQLLYVGATRARHNVYYI

SEQ ID NO: 21 - protein, 442 aa, unidentified
  source 1..442: note = Shigella phage SP18
MIKFEDLNTGQKEAFDYITEAIQRRSGECITLNGPAGTGKTTLTKFVIDHLVRNGVMGIVLAAPTHQAKKVLSKLSGQTANTIHSILKINPTTYEDQNIFEQREMPDMSKCNVLVCDEASMYDGSLFKIICNSVPEWCTILGIGDMHQLQPVDPGSTQQKISPFFTHPKFKQIHLTEVMRSNAPIIEVATEIRNGGWFRDCMYDGHGVQGFTSQTALKDFMVNYFGIVKDADMLMENRMYAYTNKSVEKLNNIIRRKLYETDKAFLPYEVLVMQEPHMKELEFEGKKFSETIFNNGQLVRIKDCKYTSTILRCKGESHQLVINYWDLEVESIDEDEEYQVDRIKVLPEDQQPKFQAYLAKVADTYKQMKAAGKRPEWKDFWKARRTFLKVRALPVSTIHKAQGVSVDKAFIYTPCIHMAEASLASQLAYVGITRARYDAYYV

SEQ ID NO: 22 - protein, 439 aa, unidentified
  source 1..439: note = Yersinia phage phiR1-RT
MITYDDLTDGQKSAFDNTMEAIKNKKGHITINGPAGTGKTTLTKFIIDHLIKTGEAGIILCAPTHQAKKVLSKLSGMDASTIHSVLKINPTTYEENQIFEQREVPDLAACRVLICDEASFYDRKLFGIILATVPSWCTVIALGDKDQLRPVTPGESEQQLSPFFSHAKFKQVHLTEIKRSNGPIIQVATDIRNGGWLSENIVDGEGVHAFNSNTALKDFMIRYFDVVKTADDLIESRMLAYTNKSVDKLNGIIRRKLYETDKPFINGEVLVMQEPLMKELEFDGKKFHEIVFNNGQLVKILYASETSTFISARNVPGEYMIRYWNLEVETADSDDDYATSQIQVICDPAEMTKFQMFLAKTADTYKNSGVKAYWKDFWSVKNKFKKVKALPVSTIHKSQGCTVNNTFLYTPCIHMADAQLAKQLLYVGATRARTNLYYI

SEQ ID NO: 23 - protein, 441 aa, unidentified
  source 1..441: note = Salmonella phage S16
MITFEQLTSGQKLAFDETIRAIKEKKNHVTINGPAGTGKTTLTKFIMEHLVSTGETGIILTAPTHAAKKVLTKLSGMEANTIHKILKINPTTYEESMLFEQKEVPDLASCRVLICDEASMWDRKLFKILMASIPKWCTIVAIGDVAQIRPVDPGETEAHISPFFIHKDFKQLNLTEVMRSNAPIIDVATDIRNGSWIYEKTVDGHGVHGFTSTTALKDFMMQYFSIVKSPEDLFENRMLAFTNKSVDKLNSIIRRRLYQTEEAFVVGEVIVMQEPLMRELVFEGKKFHETLFTNGQYVRILSADYTSSFLGAKGVSGEHLIRHWVLDVETYDDEEYAREKINVISDEQEMNKFQFFLAKTADTYKNWNKGGKAPWSEFWDAKRKFHKVKALPCSTFHKAQGISVDSSFIYTPCIHVSSDNKFKLELLYVGATRGRHDVFFV

SEQ ID NO: 24 - protein, 65 aa, synthetic construct
  REGION 1..65: note = Synthetic polypeptide
GTGSGAWKEWLERKVGEGRARRLIEYFGSAGEVGKLVENAEVSKLLEVPGIGDEAVARLVPGGSS

SEQ ID NO: 25 - protein, 299 aa, unidentified
  source 1..299: note = Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 26 - protein, 232 aa, unidentified
  source 1..232: note = Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 27 - protein, 324 aa, unidentified
  source 1..324: note = Herpes virus 1
MDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKHHHHHH

SEQ ID NO: 28 - protein, 251 aa, synthetic construct
  REGION 1..251: note = Synthetic polypeptide
MFKIVYPNAKDFFSFINSITNVTDSIILNFTEDGIFSRHLTEDKVLMAIMRIPKDVLSEYSIDSPTSVKLDVSSVKKILSKASSKKATIELTETDSGLKIIIRDEKSGAKSTIYIKAEKGQVEQLTEPKVNLAVNFTTDESVLNVIAADVTLVGEEMRISTEEDKIKIEAGEEGKRYVAFLMKDKPLKELSIDTSASSSYSAEMFKDAVKGLRGFSAPTMVSFGENLPMKIDVEAVSGGHMIFWIAPRLLE

SEQ ID NO: 29 - protein, 245 aa, synthetic construct
  REGION 1..245: note = Synthetic polypeptide
MKAKVIDAVSFSYILRTVGDFLSEANFIVTKEGIRVSGIDPSRVVFLDIFLPSSYFEGFEVSQEKEIIGFKLEDVNDILKRVLKDDTLILSSNESKLTLTFDGEFTRSFELPLIQVESTQPPSVNLEFPFKAQLLTITFADIIDELSDLGEVLNIHSKENKLYFEVIGDLSTAKVELSTDNGTLLEASGADVSSSYGMEYVANTTKMRRASDSMELYFGSQIPLKLRFKLPQEGYGDFYIAPRAD

SEQ ID NO: 30 - protein, 246 aa, synthetic construct
  REGION 1..246: note = Synthetic polypeptide
MKVVYDDVRVLKDIIQALARLVDEAVLKFKQDSVELVALDRAHISLISVNLPREMFKEYDVNDEFKFGFNTQYLMKILKVAKRKEAIEIASESPDSVIINIIGSTNREFNVRNLEVSEQEIPEINLQFDISATISSDGFKSAISEVSTVTDNVVVEGHEDRILIKAEGESEVEVEFSKDTGGLQDLEFSKESKNSYSAEYLDDVLSLTKLSDYVKISFGNQKPLQLFFNMEGGGKVTYLLAPKVLE

SEQ ID NO: 31 - protein, 608 aa, unidentified
  source 1..608: note = Bacillus subtilis phage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 32 - protein, 318 aa, unidentified
  source 1..318: note = Herpes virus 1
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 33 - protein, 233 aa, unidentified
  source 1..233: note = Bacteriophage RB69
KGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAAS

SEQ ID NO: 34 - protein, 210 aa, unidentified
  source 1..210: note = Bacteriophage T7
AKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPR

SEQ ID NO: 35 - protein, 99 aa, Halorubrum lacusprofundi
SGEELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 36 - protein, 102 aa, Haloferax volcanii
SGEELLDLAGVRGVGRKRARRLFEAGVETRADLREADKPRVLAALRGRRKTAENILEAAGRKDPSMDAVDEDDAPDDAVPDDAGFETAKERADQQASLGDFE

SEQ ID NO: 37 - protein, 55 aa, synthetic construct
  REGION 1..55: note = Synthetic polypeptide
WKEWLERKVGEGRARRLIEYFGSAGEVGKLVENAEVSKLLEVPGIGDEAVARLVP

SEQ ID NO: 38 - protein, 107 aa, synthetic construct
  REGION 1..107: note = Synthetic polypeptide
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 39 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 40 - protein, 123 aa, unidentified
  source 1..123: note = Bacillus subtilis phage phi29
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 41 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 42 - protein, 301 aa, unidentified
  source 1..301: note = Enterobacteria phage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 43 - protein, 177 aa, synthetic construct
  REGION 1..177: note = Synthetic polypeptide
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMAFAAAIPF

SEQ ID NO: 44 - protein, 177 aa, synthetic construct
  REGION 1..177: note = Synthetic polypeptide
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMNFGGNIPF

SEQ ID NO: 45 - protein, 152 aa, synthetic construct
  REGION 1..152: note = Synthetic polypeptide
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQ

SEQ ID NO: 46 - protein, 117 aa, synthetic construct
  REGION 1..117: note = Synthetic polypeptide
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQG

SEQ ID NO: 47 - protein, 984 aa, Methanopyrus kandleri
MALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 48 - protein, 299 aa, Methanopyrus kandleri
SGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 49 - protein, 853 aa, Escherichia coli
MSAIENFDAHTPMMQQYLRLKAQHPEILLFYRMGDFYELFYDDAKRASQLLDISLTKRGASAGEPIPMAGIPYHAVENYLAKLVNQGESVAICEQIGDPATSKGPVERKVVRIVTPGTISDEALLQERQDNLLAAIWQDSKGFGYATLDISSGRFRLSEPADRETMAAELQRTNPAELLYAEDFAEMSLIEGRRGLRRRPLWEFEIDTARQQLNLQFGTRDLVGFGVENAPRGLCAAGCLLQYAKDTQRTTLPHIRSITMEREQDSIIMDAATRRNLEITQNLAGGAENTLASVLDCTVTPMGSRMLKRWLHMPVRDTRVLLERQQTIGALQDFTAGLQPVLRQVGDLERILARLALRTARPRDLARMRHAFQQLPELRAQLETVDSAPVQALREKMGEFAELRDLLERAIIDTPPVLVRDGGVIASGYNEELDEWRALADGATDYLERLEVRERERTGLDTLKVGFNAVHGYYIQISRGQSHLAPINYMRRQTLKNAERYIIPELKEYEDKVLTSKGKALALEKQLYEELFDLLLPHLEALQQSASALAELDVLVNLAERAYTLNYTCPTFIDKPGIRITEGRHPVVEQVLNEPFIANPLNLSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWACAENLANKIKALTLFATHYFELTQLPEKMEGVANVHLDALEHGDTIAFMHSVQDGAASKSYGLAVAALAGVPKEVIKRARQKLRELESISPNAAATQVDGTQMSLLSVPEETSPAVEALENLDPDSLTPRQALEWIYRLKSLV

SEQ ID NO: 50 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 51 - protein, 99 aa, Sulfolobus solfataricus
  source 1..99: note = P2
EKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 52 - protein, 88 aa, Sulfolobus solfataricus
  source 1..88: note = P2
TEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 53 - protein, 107 aa, Escherichia coli
AQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 54 - protein, 237 aa, unidentified
  source 1..237: note = Enterobacteria phage lambda
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 55 - protein, 60 aa, unidentified
  source 1..60: note = Crenarchaea
MSSGKKPVKVKTPAGKEAELVPEKVWALAPKGRKGVKIGLFKDPETGKYFRHKLPDDYPI

SEQ ID NO: 56 - protein, 136 aa, Homo sapiens [sequence omitted]

SEQ ID NO: 57 - protein, 89 aa, unidentified
  source 1..89: note = Enterobacteria phage T4
MAKKEMVEFDEAIHGEDLAKFIKEASDHKLKISGYNELIKDIRIRAKDELGVDGKMFNRLLALYHKDNRDVFEAETEEVVELYDTVFSK

SEQ ID NO: 58 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 59 - protein, 375 aa, unidentified
  source 1..375: note = Citromicrobium bathyomarinum JL354
MKATIERATLLRCLSHVQSVVERRNTIPILSNVLIDADAGGGVKVMATDLDLQVVETMTAASVESAGAITVSAHLLFDIARKLPDGSQVSLETADNRMVVKAGRSRFQLPTLPRDDFPVIVEGELPTSFELPARELAEMIDRTRFAISTEETRYYLNGIFLHVSDEARPVLKAAATDGHRLARYTLDRPEGAEGMPDVIVPRKAVGELRKLLEEALDSNVQIDLSASKIRFALGGEGGVVLTSKLIDGTFPDYSRVIPTGNDKLLRLDPKAFFQGVDRVATIATEKTRAVKMGLDEDKVTLSVTSPDNGTAAEEIAAEYKAEGFEIGFNANYLKDILGQIDSDTVELHLADAGAPTLIRRDENSPALYVLMPMRV

SEQ ID NO: 60 - DNA, 12 bp, synthetic construct
  misc_feature 1..12: note = Synthetic polynucleotide
  source 1..12: mol_type = other DNA
tttttttttttt

SEQ ID NO: 61 - DNA, 3667 bp, synthetic construct
  misc_feature 1..3667: note = Synthetic polynucleotide
  source 1..3667: mol_type = other DNA
ggttgtttctgttggtgctgatattgcggcgtctgcttgggtgtttaacctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcatgtaggacgtcctgtcgctgtgtctcggacactgattgacacggtttagtagagc

SEQ ID NO: 62 - DNA, 3617 bp, synthetic construct
  misc_feature 1..3617: note = Synthetic polynucleotide
  source 1..3617: mol_type = other DNA
ttttttttttttttttttttttttttttcgagacacagcgacaggacgtcctacatgcttacggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggca

SEQ ID NO: 63 - DNA, 68 bp, synthetic construct
  misc_feature 1..68: note = Synthetic polynucleotide
  source 1..68: mol_type = other DNA
ggttaaacacccaagcagacgccgcaatatcagcaccaacagaaacaacctttgaggcgagcggtcaa

SEQ ID NO: 64 - DNA, 15 bp, synthetic construct
  misc_feature 1..15: note = Synthetic polynucleotide
  source 1..15: mol_type = other DNA
ttgaccgctcgcctc

SEQ ID NO: 65 - DNA, 786 bp, Escherichia coli
  source 1..786: mol_type = genomic DNA
tgtctgaccgcaccgccgaaagaagcggcacgtccgaccctgatgccgcgtgcacagtcttataaagatctgacccatctgccggctccgacgggcaaaatttttgttagcgtctataacatccaggacgaaaccggtcaatttaaaccgtacccggcgagtaatttctccacggccgttccgcagagtgcaaccgctatgctggtcacggcactgaaagattcccgttggttcattccgctggaacgccagggcctgcaaaacctgctgaatgaacgtaaaattatccgcgcagctcaggaaaacggtaccgtggccattaacaatcgtattccgctgcaaagcctgaccgccgcaaacatcatggttgaaggctctatcatcggttacgaatcaaacgtcaaatcgggcggtgtgggcgcacgttattttggcattggtgctgatacccagtaccaactggaccagatcgcagttaacctgcgcgtggttaatgtcagcaccggcgaaattctgagctctgtgaataccagcaaaacgatcctgtcttacgaagtgcaggctggtgtttttcgtttcattgattatcaacgcctgctggaaggcgaagtcggttacacctcaaacgaaccggtgatgctgtgtctgatgtcggcgattgaaacgggtgttattttcctgatcaatgatggcatcgaccgtggtctgtgggatctgcagaacaaagccgaacgtcaaaatgacattctggtgaaataccgccacatgagtgttccgccggaatcc

SEQ ID NO: 66 - protein, 262 aa, Escherichia coli
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 67 - protein, 10 aa, synthetic construct
  REGION 1..10: note = Synthetic polypeptide
SAWSHPQFEK

SEQ ID NO: 68 - RNA, 1653 nt, synthetic construct
  misc_feature 1..1653: note = Synthetic polynucleotide
  source 1..1653: mol_type = other RNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 69 - DNA, 10 bp, synthetic construct
  misc_feature 1..10: note = Synthetic polynucleotide
  source 1..10: mol_type = other DNA
ccccccccca

SEQ ID NO: 70 - DNA, 27 bp, synthetic construct
  misc_feature 1..27: note = Synthetic polynucleotide
  source 1..27: mol_type = other DNA
ggttgtttctgttggtgctgatattgc

SEQ ID NO: 71 - DNA, 28 bp, synthetic construct
  misc_feature 1..28: note = Synthetic polynucleotide
  source 1..28: mol_type = other DNA
gcaatatcagcaccaacagaaacaacct

SEQ ID NO: 72 - DNA, 48 bp, synthetic construct
  misc_feature 1..48: note = Synthetic polynucleotide
  source 1..48: mol_type = other DNA
cgttctgtttatgtttcttggacactgattgacacggtttagtagaac

SEQ ID NO: 73 - DNA, 51 bp, synthetic construct
  misc_feature 1..51: note = Synthetic polynucleotide
  source 1..51: mol_type = other DNA
cccccccccacccccccccacccccccccaagaaacataaacagaacgttt

(19) United States
(12) Patent Application Publication
(10) Pub. No.: US 2024/0287485 A1
(43) Pub. Date: Aug. 29, 2024

(54) MODIFIED ENZYMES

(71) Applicant: Oxford Nanopore Technologies PLC, Oxford (GB)

(72) Inventors: Andrew John Heron, Oxford (GB); Rebecca Victoria Bowen, Oxford (GB); Mark John Bruce, Oxford (GB); Lakmal Nishantha Jayasinghe, Oxford (GB); Joseph Hargreaves Lloyd, Oxford (GB); Szabolcs Soeroes, Oxford (GB); Elizabeth Jayne Wallace, Oxford (GB); Christopher Peter Youd, Oxford (GB)

(73) Assignee: Oxford Nanopore Technologies PLC, Oxford (GB)

(21) Appl. No.: 18/596,176

(22) Filed: Mar. 5, 2024

Related U.S. Application Data
(63) Continuation of application No. 17/500,831, filed on Oct. 13, 2021, now Pat. No. 11,965,183.
Application No. 17/500,831 is a division of application No. 15/517,592, filed on Apr. 7, 2017, now Pat. No. 11,180,741.
Application No. 15/517,592 is a continuation-in-part of application No. PCT/GB2015/051291, filed on May 1, 2015.

(30) Foreign Application Priority Data
Oct. 7, 2014 (GB) 1417712.5

(51) Int. Cl. C12N 9/14; C12Q 1/68; C12Q 1/6869

(52) U.S. Cl. CPC C12N 9/14; C12Q 1/68; C12Q 1/6869; C12Y 306/04012

(57) ABSTRACT

The invention relates to modified Dda helicases which can be used to control the movement of polynucleotides and are particularly useful for sequencing polynucleotides.

### RELATED APPLICATIONS

This application is a continuation of U.S. patent application U.S. Ser. No. 17/500,831, filed Oct. 13, 2021, which is a divisional of U.S. patent application U.S. Ser. No. 15/517,592, filed Apr. 7, 2017, which is a national stage filing under 35 U.S.C. § 371 of PCT International Application No. PCT/GB2015/052916, which has an international filing date of Oct. 6, 2015; is a continuation-in-part of PCT International Application No. PCT/GB2015/051291, which has an international filing date of May 1, 2015; and claims foreign priority benefits under 35 U.S.C. § 119(a)-(d) or 35 U.S.C. § 365(b) of British application number 1417712.5, filed Oct. 7, 2014, the contents of each of which are herein incorporated by reference in their entireties.

### REFERENCE TO AN ELECTRONIC SEQUENCE LISTING

The contents of the electronic sequence listing (O036670051US02-SUBSEQ-CBD.xml; Size: 106,716 bytes; and Date of Creation: May 6, 2024) is herein incorporated by reference in its entirety.

### FIELD OF THE INVENTION

The invention relates to modified Dda helicases which can be used to control the movement of polynucleotides and are particularly useful for sequencing polynucleotides.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the “strand sequencing” method, a single polynucleotide strand is passed through the pore and the identities of the nucleotides are derived. Strand sequencing can involve the use of a nucleotide handling protein, such as a helicase, to control the movement of the polynucleotide through the pore.

### SUMMARY OF THE INVENTION

The inventors have surprisingly identified specific Dda mutants which have an improved ability to control the movement of a polynucleotide through a pore. The mutants of the invention display reduced forward slipping. This is a phenomenon where the DNA moves forwards relative to the pore by at least 4 consecutive nucleotides and typically by more than 10 consecutive nucleotides. Slipping forward may involve movement forward of 100 consecutive nucleotides or more and this may happen more than once for each polynucleotide. Slipping forward can be problematic for polynucleotide sequencing. The mutants identified by the inventors typically comprise a combination of mutations, namely (1) one or more substitutions of the amino acids which interact with nucleotides in single stranded DNA (ssDNA) and (2) one or more modifications in the part of the mutant which interacts with a transmembrane pore.

Accordingly, the invention provides a DNA-dependent ATPase (Dda) helicase in which (a) at least one amino acid which interacts with one or more nucleotides in single stranded DNA (ssDNA) is substituted and (b) the part of the helicase which interacts with a transmembrane pore comprises one or more modifications, wherein the helicase has the ability to control the movement of a polynucleotide.

The invention also provides:
a construct comprising a helicase of the invention and an additional polynucleotide binding moiety, wherein the helicase is attached to the polynucleotide binding moiety and the construct has the ability to control the movement of a polynucleotide;
a polynucleotide comprising a sequence which encodes a helicase of the invention or a construct of the invention;
a vector which comprises a polynucleotide of the invention operably linked to a promoter;
a host cell comprising a vector of the invention;
a method of making a helicase of the invention or a construct of the invention, which comprises expressing a polynucleotide of the invention, transfecting a cell with a vector of the invention or culturing a host cell of the invention;
a method of controlling the movement of a polynucleotide, comprising contacting the polynucleotide with a helicase of the invention or a construct of the invention and thereby controlling the movement of the polynucleotide;
a method of characterising a target polynucleotide, comprising (a) contacting the target polynucleotide with a transmembrane pore and a helicase of the invention or a construct of the invention such that the helicase controls the movement of the target polynucleotide through the pore and (b) taking one or more measurements as the polynucleotide moves with respect to the pore wherein the measurements are indicative of one or more characteristics of the target polynucleotide and thereby characterising the target polynucleotide;
a method of forming a sensor for characterising a target polynucleotide, comprising forming a complex between (a) a pore and (b) a helicase of the invention or a construct of the invention and thereby forming a sensor for characterising the target polynucleotide;
sensor for characterising a target polynucleotide, comprising a complex between (a) a pore and (b) a helicase of the invention or a construct of the invention;
use of a helicase of the invention or a construct of the invention to control the movement of a target polynucleotide through a pore;
a kit for characterising a target polynucleotide comprising (a) a pore and (b) a helicase of the invention or a construct of the invention;
an apparatus for characterising target polynucleotides in a sample, comprising (a) a plurality of pores and (b) a plurality of helicases of the invention or a plurality of constructs of the invention; and
a series of two or more helicases attached to a polynucleotide, wherein at least one of the two or more helicases is a helicase of the invention.

### DESCRIPTION OF THE FIGURES

FIG. 1 shows the three different initial simulation orientations of T4 Dda—E94C/A360C/C109A/C136A (SEQ ID NO: 8 with mutations E94C/A360C/C109A/C136A) with respect to either MspA—(G75S/G77S/L88N/D90N/D91N/D118R/Q126R/D134R/E139K)8 (SEQ ID NO: 2 with mutations G75S/G77S/L88N/D90N/D91N/D118R/Q126R/D134R/E139K=MspA mutant 1) or MspA—((Del-L74/G75/D118/L119)D56N/E59R/L88N/D90N/D91N/Q126R/D134R/E139K)8 (SEQ ID NO: 2 with mutations D56N/E59R/L88N/D90N/D91N/Q126R/D134R/E139K and deletion of the amino acids L74/G75/D118/L119=MspA mutant 2). The difference between run 1 and run 2 was that both the enzyme and pore had different side chain conformations despite the pore and enzyme being in the same position. In run three the enzyme has been tilted slightly with respect to the nanopore.

FIG. 2 shows a plot (y-axis label=number of pore/enzyme contacts, x-axis label=pore amino acid residue number) of the interaction points of the nanopore MspA mutant 1 with enzyme mutant 1. Each row of the plot shows the interaction points for the different enzyme/nanopore orientations e.g. runs 1-3.

FIG. 3 shows a plot (y-axis label=number of pore/enzyme contacts, x-axis label=enzyme amino acid residue number) of the interaction points of the enzyme mutant 1 with MspA mutant 1. Each row of the plot shows the interaction points for the different enzyme/nanopore orientations e.g. runs 1-3.

FIG. 4 shows a plot (y-axis label=number of pore/enzyme contacts, x-axis label=pore amino acid residue number) of the interaction points of the nanopore MspA mutant 2 with enzyme mutant 1. Each row of the plots shows the interaction points for the different enzyme/nanopore orientations e.g. runs 1-3.

FIG. 5 shows a plot (y-axis label=number of pore/enzyme contacts, x-axis label=enzyme amino acid residue number) of the interaction points of the enzyme mutant 1 with MspA mutant 2. Each row of the plot shows the interaction points for the different enzyme/nanopore orientations e.g. runs 1-3.

FIG. 6 (A) shows two regions of a plot (y-axis label=pore amino acid residue number, x-axis label=enzyme amino acid residue number) which shows which amino acids in the pore (MspA mutant 2) interact with particular amino acids in the enzyme (enzyme mutant 1) from run 1. FIG. 6 (B) shows a region of a plot (y-axis label (a1)=pore amino acid residue number, y-axis label (a2)=number of pore/enzyme contacts, x-axis label=enzyme amino acid residue number) which shows which amino acids in the pore (MspA mutant 2) interact with particular amino acids in the enzyme (enzyme mutant 1) from run 3. The grey bands in the plots indicate an interaction between amino acids. The darkness of the grey band corresponds to the number of interactions between enzyme/pore, with dark grey=many interactions and light grey=fewer interactions. The first amino acid in each box corresponds to the interacting amino acid in the MspA mutant 2 and the second amino acid corresponds to the interacting amino acids in enzyme mutant 1.

FIG. 7 shows DNA construct X used in Example 3. Section A corresponded to thirty iSpC3 spacers. Section B corresponded to SEQ ID NO: 60. Label C corresponded to the enzyme mutant used in the experiment. Section D corresponded to four iSp18 spacers. Section E corresponded to SEQ ID NO: 61. Section F corresponded to four iSpC3 spacers. Section G corresponded to SEQ ID NO: 62. Section H corresponded to SEQ ID NO: 63. Section I corresponded to SEQ ID NO: 64. Section J corresponded to a 3′ cholesterol.

FIG. 8 shows example current traces (y-axis label=Current (pA), x-axis label=Time (s) for all three traces) of when a helicase (T4 Dda—E94C/F98W/C109A/C136A/K194L/A360C (SEQ ID NO: 8 with mutations E94C/F98W/C109A/C136A/K194L/A360C and then (ΔM1)G1)) controlled the translocation of the DNA construct X through the CsgG-Eco nanopore (CsgG-Eco-(Y51T/F56Q)-StrepII(C))9 (SEQ ID NO: 66 with mutations Y51T/F56Q where StepII(C) is SEQ ID NO: 67 and is attached at the C-terminus). Sections B and C show zoomed in regions of current trace A.

FIG. 9 shows an example current trace (y-axis label=Current (pA), x-axis label=Time (s)) of when a helicase (T4 Dda—E94C/F98W/C109A/C136A/K194L/A360C (SEQ ID NO: 8 with mutations E94C/F98W/C109A/C136A/K194L/A360C and then (ΔM1)G1)) controlled the translocation of the DNA/RNA construct Y through an MspA nanopore. The region labelled 1 corresponds to an RNA region and the region labelled 2 corresponds to a DNA region.

FIGS. 10A-10E show a sequence alignment of Dda helicases, SEQ ID NOs: 8-23.

FIG. 11A shows an example of complementary polynucleotides that may be used to bind helicase.

FIG. 11B shows an example of DNA that could be used for helicase-Phi29 constructs.

### DESCRIPTION OF THE SEQUENCE LISTING

SEQ ID NO: 1 shows the codon optimised polynucleotide sequence encoding the wild-type MspA monomer. This mutant lacks the signal sequence.

SEQ ID NO: 2 shows the amino acid sequence of the mature form of the wild-type MspA monomer. This mutant lacks the signal sequence.

SEQ ID NO: 3 shows the polynucleotide sequence encoding one monomer of α-hemolysin-E111N/K147N ((α-HL-NN; Stoddart et al., PNAS, 2009; 106(19): 7702-7707).

SEQ ID NO: 4 shows the amino acid sequence of one monomer of α-HL-NN.

SEQ ID NOs: 5 to 7 show the amino acid sequences of MspB, C and D.

SEQ ID NOs: 8 to 23 show the amino acid sequences of the Dda helicases shown in Tables 1 and 2.

SEQ ID NO: 24 shows the amino acid sequence of a preferred HhH domain.

SEQ ID NO: 25 shows the amino acid sequence of the ssb from the bacteriophage RB69, which is encoded by the gp32 gene.

SEQ ID NO: 26 shows the amino acid sequence of the ssb from the bacteriophage T7, which is encoded by the gp2.5 gene.

SEQ ID NO: 27 shows the amino acid sequence of the UL42 processivity factor from Herpes virus 1.

SEQ ID NO: 28 shows the amino acid sequence of subunit 1 of PCNA.

SEQ ID NO: 29 shows the amino acid sequence of subunit 2 of PCNA.

SEQ ID NO: 30 shows the amino acid sequence of subunit 3 of PCNA.

SEQ ID NO: 31 shows the amino acid sequence of Phi29 DNA polymerase.

SEQ ID NO: 32 shows the amino acid sequence (from 1 to 319) of the UL42 processivity factor from the Herpes virus 1.

SEQ ID NO: 33 shows the amino acid sequence of the ssb from the bacteriophage RB69, i.e. SEQ ID NO: 25, with its C terminus deleted (gp32RB69CD).

SEQ ID NO: 34 shows the amino acid sequence (from 1 to 210) of the ssb from the bacteriophage T7 (gp2.5T7-R211Del). The full length protein is shown in SEQ ID NO: 96.

SEQ ID NO: 35 shows the amino acid sequence of the 5^{th} domain of Hel308 Hla.

SEQ ID NO: 36 shows the amino acid sequence of the 5^{th} domain of Hel308 Hvo.

SEQ ID NO: 37 shows the amino acid sequence of the (HhH)2 domain.

SEQ ID NO: 38 shows the amino acid sequence of the (HhH)2-(HhH)2 domain.

SEQ ID NO: 39 shows the amino acid sequence of the human mitochondrial SSB (HsmtSSB).

SEQ ID NO: 40 shows the amino acid sequence of the p5 protein from Phi29 DNA polymerase.

SEQ ID NO: 41 shows the amino acid sequence of the wild-type SSB from E. coli.

SEQ ID NO: 42 shows the amino acid sequence of the ssb from the bacteriophage T4, which is encoded by the gp32 gene.

SEQ ID NO: 43 shows the amino acid sequence of EcoSSB-CterAla.

SEQ ID NO: 44 shows the amino acid sequence of EcoSSB-CterNGGN.

SEQ ID NO: 45 shows the amino acid sequence of EcoSSB-Q152del.

SEQ ID NO: 46 shows the amino acid sequence of EcoSSB-G117del.

SEQ ID NO: 47 shows the amino acid sequence of Topoisomerase V Mka (Methanopyrus Kandleri).

SEQ ID NO: 48 shows the amino acid sequence of domains H-L of Topoisomerase V Mka (Methanopyrus Kandleri).

SEQ ID NO: 49 shows the amino acid sequence of Mutant S (Escherichia coli).

SEQ ID NO: 50 shows the amino acid sequence of Sso7d (Sulfolobus solfataricus).

SEQ ID NO: 51 shows the amino acid sequence of Sso10b1 (Sulfolobus solfataricus P2).

SEQ ID NO: 52 shows the amino acid sequence of Sso10b2 (Sulfolobus solfataricus P2).

SEQ ID NO: 53 shows the amino acid sequence of Tryptophan repressor (Escherichia coli).

SEQ ID NO: 54 shows the amino acid sequence of Lambda repressor (Enterobacteria phage lambda).

SEQ ID NO: 55 shows the amino acid sequence of Cren7 (Histone crenarchaea Cren7 Sso).

SEQ ID NO: 56 shows the amino acid sequence of human histone (Homo sapiens).

SEQ ID NO: 57 shows the amino acid sequence of dsbA (Enterobacteria phage T4).

SEQ ID NO: 58 shows the amino acid sequence of Rad51 (Homo sapiens).

SEQ ID NO: 59 shows the amino acid sequence of PCNA sliding clamp (Citromicrobium bathyomarinum JL354).

SEQ ID NOs: 60 to 64 show a polynucleotide sequences used in Example 3.

SEQ ID NO: 65 shows the codon optimised polynucleotide sequence encoding the wild-type CsgG monomer from Escherichia coli Str. K-12 substr. MC4100. This monomer lacks the signal sequence.

SEQ ID NO: 66 shows the amino acid sequence of the mature form of the wild-type CsgG monomer from Escherichia coli Str. K-12 substr. MC4100. This monomer lacks the signal sequence. The abbreviation used for this CsgG=CsgG-Eco.

SEQ ID NO: 67 shows the amino acid sequence of StepII(C).

SEQ ID NOs: 68 to 73 shows the polynucleotide sequences used in Example 5.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a helicase” includes “helicases”, reference to “a modification” includes two or more such modifications, reference to “a transmembrane protein pore” includes two or more such pores, and the like.

All publications, patents and patent applications cited herein, whether supra or infra, are hereby incorporated by reference in their entirety.

### Modified Dda Helicases

The present invention provides a modified Dda helicase. The one or more specific modifications are discussed in more detail below. Modifications according to the invention include one or more substitutions as discussed below.

Characterisation, such as sequencing, of a polynucleotide using a transmembrane pore and helicase typically involves analysing polymer units made up of k nucleotides where k is a positive integer (i.e. ‘k-mers’). This is discussed in International Application No. PCT/GB2012/052343 (published as WO 2013/041878). As the target polynucleotide moves with respect to, or through the pore, different k-mers within the polynucleotide are analysed, typically by measuring the current flowing through the pore. The movement of the polynucleotide with respect to, such as through, the pore can be viewed as movement from one k-mer to another or from k-mer to k-mer.

The modified helicases of the invention provide more consistent movement of the target polynucleotide with respect to, such as through, the transmembrane pore. The helicases preferably provide more consistent movement from one k-mer to another or from k-mer to k-mer as the target polynucleotide moves with respect to, such as through, the pore. The helicases allow the target polynucleotide to move with respect to, such as through, the transmembrane pore more smoothly. The helicases preferably provide more regular or less irregular movement of the target polynucleotide with respect to, such as through, the transmembrane pore.

The modification(s), particularly the substitution of one or more amino acids which interact with one or more nucleotides in ssDNA), allow the modified helicase to display reduced forward slipping. This is a phenomenon where the DNA moves forwards relative to the pore by at least 4 consecutive nucleotides and typically by more than 10 consecutive nucleotides. Slipping forward may involve movement forward of 100 consecutive nucleotides or more and this may happen more than once for each polynucleotide. Slipping forward can be problematic for polynucleotide sequencing.

The modification(s) typically reduces the frequency of forward slipping displayed by the helicase by at least 10%, at least 20%, at least 30%, at least 40%, at least 50%, at least 60%, at least 70%, at least 80% or at least 90%. The modification(s) typically abolishes forward slipping, i.e. reduces the frequency of forward slipping displayed by the helicase by 100%. The modification(s) typically reduces the length of forward slipping displayed by the helicase to 10 nucleotides or fewer, such as 9 nucleotides or fewer, 8 nucleotides or fewer, 7 nucleotides or fewer, 6 nucleotides or fewer, 5 nucleotides or fewer, 4 nucleotides or fewer, 3 nucleotides or fewer, 2 nucleotides or fewer or 1 nucleotide. The modification(s) preferably reduce the frequency and length of forward slipping displayed by the helicase.

Forward slipping can be measured using any method known in the art. The ability of a helicase to control the movement of a polynucleotide and the incidence of forward slipping is typically assayed in a nanopore system, such as the ones described below. The ability of a helicase to control the movement of a polynucleotide and the incidence of forward slipping can be determined as described in the Examples.

The modifications (s), particularly the modification(s) in the part of the helicase which interacts with a transmembrane pore, typically reduce the noise associated with the movement of the target polynucleotide with respect to, such as through, the transmembrane pore. Unwanted movement of the target polynucleotide in any dimension as a k-mer is being analysed typically results in noise in the current signature or level for the k-mer. The helicases of the invention may reduce this noise by reducing unwanted movement associated with one or more k-mers, such as each k-mer, in the target polynucleotide. The helicases may reduce the noise associated with the current level or signature for one or more k-mers, such as each k-mer, in the target polynucleotide.

In a preferred embodiment, the target polynucleotide is double stranded and the helicase reduces the noise associated with the movement of the complement strand to a greater degree than it reduces the noise associated with the movement of the template strand and/or increases the consistency of the movement of the complement strand to a greater degree than it increases the consistency of the movement of the template strand. This is advantageous for strand sequencing of double stranded target polynucleotides. The two stands of the double stranded polynucleotide are preferably linked by a bridging moiety, such as a hairpin loop or hairpin loop adaptor. This is discussed in more detail below. In other words, the modified helicases of the invention are better at controlling the movement of a polynucleotide. The extent to which the helicases can control the movement of a polynucleotide is typically altered by the modifications as discussed in more detail below.

The helicase of the invention is modified. The modified helicase is typically modified compared with the corresponding wild-type helicase or natural helicase. The helicase of the invention is artificial or non-natural.

A modified helicase of the invention is a useful tool for controlling the movement of a polynucleotide during Strand Sequencing. The helicase can control the movement of DNA in at least two active modes of operation (when the helicase is provided with all the necessary components to facilitate movement e.g. ATP and Mg²⁺) and one inactive mode of operation (when the helicase is not provided with the necessary components to facilitate movement). When provided with all the necessary components to facilitate movement the helicase moves along the DNA in the 5′-3′ direction, but the orientation of the DNA in the nanopore (dependent on which end of the DNA is captured) means that the enzyme can be used to either move the DNA out of the nanopore against the applied field, or move the DNA into the nanopore with the applied field. When the 3′ end of the DNA is captured the helicase works against the direction of the field applied by the voltage, pulling the threaded DNA out of the nanopore and into the cis chamber. However, when the DNA is captured 5′-down in the nanopore, the helicase works with the direction of the field applied by the voltage, pushing the threaded DNA into the nanopore and into the trans chamber. When the helicase is not provided with the necessary components to facilitate movement it can bind to the DNA and act as a brake slowing the movement of the DNA when it is pulled into the pore by the applied field. In the inactive mode it does not matter whether the DNA is captured either 3′ or 5′ down, it is the applied field which pulls the DNA into the nanopore towards the trans side with the enzyme acting as a brake. When in the inactive mode the movement control of the DNA by the helicase can be described in a number of ways including ratcheting, sliding and braking.

A problem which occurs in sequencing polynucleotides, particularly those of 500 nucleotides or more, is that the molecular motor which is controlling the movement of the polynucleotide may disengage from the polynucleotide. This allows the polynucleotide to be pulled through the pore rapidly and in an uncontrolled manner in the direction of the applied field. A modified helicase of the invention is less likely to unbind or disengage from the polynucleotide being sequenced. The modified helicase can provide increased read lengths of the polynucleotide as they control the movement of the polynucleotide through a nanopore. The ability to move an entire polynucleotide through a nanopore under the control of a modified helicase of the invention allows characteristics of the polynucleotide, such as its sequence, to be estimated with improved accuracy and speed over known methods. This becomes more important as strand lengths increase and molecular motors are required with improved processivity. A modified helicase of the invention is particularly effective in controlling the movement of target polynucleotides of 500 nucleotides or more, for example 1000 nucleotides, 5000, 10000, 20000, 50000, 100000 or more.

A modified helicase of the invention is also a useful tool for isothermal polymerase chain reaction (PCR). In such methods, the strands of double stranded DNA are typically first separated by a helicase of the invention and coated by single stranded DNA (ssDNA)-binding proteins. In the second step, two sequence specific primers typically hybridise to each border of the DNA template. DNA polymerases may then be used to extend the primers annealed to the templates to produce a double stranded DNA and the two newly synthesized DNA products may then be used as substrates by the helicases of the invention, entering the next round of the reaction. Thus, a simultaneous chain reaction develops, resulting in exponential amplification of the selected target sequence.

The modified helicase has the ability to control the movement of a polynucleotide. The ability of a helicase to control the movement of a polynucleotide can be assayed using any method known in the art. For instance, the helicase may be contacted with a polynucleotide and the position of the polynucleotide may be determined using standard methods. The ability of a modified helicase to control the movement of a polynucleotide is typically assayed in a nanopore system, such as the ones described below and, in particular, as described in the Examples.

A modified helicase of the invention may be isolated, substantially isolated, purified or substantially purified. A helicase is isolated or purified if it is completely free of any other components, such as lipids, polynucleotides, pore monomers or other proteins. A helicase is substantially isolated if it is mixed with carriers or diluents which will not interfere with its intended use. For instance, a helicase is substantially isolated or substantially purified if it is present in a form that comprises less than 10%, less than 5%, less than 2% or less than 1% of other components, such as lipids, polynucleotides, pore monomers or other proteins.

Any Dda helicase may be modified in accordance with the invention. Preferred Dda helicases are discussed below.

Dda helicases typically comprises the following five domains: 1A (RecA-like motor) domain, 2A (RecA-like motor) domain, tower domain, pin domain and hook domain (Xiaoping He et al., 2012, Structure; 20: 1189-1200). The domains may be identified using protein modelling, x-ray diffraction measurement of the protein in a crystalline state (Rupp B (2009). Biomolecular Crystallography: Principles, Practice and Application to Structural Biology. New York: Garland Science.), nuclear magnetic resonance (NMR) spectroscopy of the protein in solution (Mark Rance; Cavanagh, John; Wayne J. Fairbrother; Arthur W. Hunt III; Skelton, NNicholas J. (2007). Protein NMR spectroscopy: principles and practice (2nd ed.). Boston: Academic Press.) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33: 307-69). Structural information of proteins determined by above mentioned methods are publicly available from the protein bank (PDB) database.

Protein modelling exploits the fact that protein structures are more conserved than protein sequences amongst homologues. Hence, producing atomic resolution models of proteins is dependent upon the identification of one or more protein structures that are likely to resemble the structure of the query sequence. In order to assess whether a suitable protein structure exists to use as a “template” to build a protein model, a search is performed on the protein data bank (PDB) database. A protein structure is considered a suitable template if it shares a reasonable level of sequence identity with the query sequence. If such a template exists, then the template sequence is “aligned” with the query sequence, i.e. residues in the query sequence are mapped onto the template residues. The sequence alignment and template structure are then used to produce a structural model of the query sequence. Hence, the quality of a protein model is dependent upon the quality of the sequence alignment and the template structure.

Simulations can be performed to assess which amino acids make contact with the nucleotides in ssDNA within the enzyme binding site. The simulations may be performed using the GROMACS package version 4.0.5, with the AMBER-99SB force field and the TIP3P water model. A preferred method is disclosed in the Examples.

### Modifications of the Invention

The helicase of the invention is one in which at least one amino acid which interacts with one or more nucleotides in single stranded DNA (ssDNA) is substituted. Any number of amino acids may substituted, such as 1 or more, 2 or more, 3 or more, 4 or more, 5 or more or 6 or more amino acids. As the helicase moves along ssDNA or as the ssDNA moves through the helicase, amino acids may sequentially interact with different nucleotides. Each amino which is substituted may interact with any number of nucleotides at a time, such as one, two, three or more nucleotides at a time. The amino acids which interact with one or more nucleotides in single stranded DNA can be identified using protein modelling as discussed above.

Base and/or Sugar Interactions

The helicase of the invention is preferably one in which at least one amino acid which interacts with the sugar and/or base of one or more nucleotides in single stranded DNA (ssDNA) is substituted with an amino acid which comprises a larger side chain (R group). Any number of amino acids may substituted, such as 1 or more, 2 or more, 3 or more, 4 or more, 5 or more or 6 or more amino acids. Each amino acid may interact with the base, the sugar or the base and the sugar. The amino acids which interact with the sugar and/or base of one or more nucleotides in single stranded DNA can be identified using protein modelling as discussed above.

Table 1 below summarises the preferred Dda helicases which may be modified in accordance with the invention.

TABLE 1

| | | | | | | Number | |
|---|---|---|---|---|---|---|---|
| | | | | | Sequence | of D/E | |
| | | | | | Identity | vs. K/R | |
| Dda Homologue | | | | | to 1993/ | amino | |
| (SEQ ID NO:) | | Habitat | Uniprot | Length | % | acids | # C |
| Rma- | Rhodothermus | Mild | D0MKQ2 | 678 | 21 | −84/+85 | 2 |
| DSM | marinus | halophile, | | | | | |
| (SEQ ID | | moderate | | | | | |
| NO: 9) | | thermophile | | | | | |
| | | >65° C. | | | | | |
| Csp | Cyanothece sp. | Marine | B1X365 | 496 | 24 | −76/+76 | 5 |
| (SEQ ID | (strain ATCC | bacterium | | | | | |
| NO: 10) | 51142) | | | | | | |
| Sru | Salinibacter | Extremely | Q2S429 | 421 | 26 | −78/+54 | 3 |
| (SEQ ID | ruber | halophilic, | | | | | |
| NO: 11) | | 35-45° C. | | | | | |
| Sgo | Sulfurimonas | Habitat: | B6BJ43 | 500 | 27 | −72/+64 | 2 |
| (SEQ ID | gotlandica GD1 | hydrothermal | | | | | |
| NO: 12) | | vents, coastal | | | | | |
| | | sediments | | | | | |
| Vph12B8 | Vibrio phage | Host found | M4MBC3 | 450 | 27 | −62 /+47 | 6 |
| (SEQ ID | henriette 12B8 | in saltwater, | | | | | |
| NO: 13) | | stomach bug | | | | | |
| Vph | Vibrio phage | Host found | I6XGX8 | 421 | 39 | −55/+45 | 5 |
| (SEQ ID | phi-pp2 | in saltwater, | | | | | |
| NO: 14) | | stomach bug | | | | | |
| Aph65 | Aeromonas | Host found | E5DRP6 | 434 | 40 | −57/+48 | 4 |
| (SEQ ID | phage 65 | in | | | | | |
| NO: 15) | | fresh/brackish | | | | | |
| | | water, | | | | | |
| | | stomach bug | | | | | |
| AphCC2 | Aeromonas | Host found | I6XH64 | 420 | 41 | −53/+44 | 4 |
| (SEQ ID | phage CC2 | in | | | | | |
| NO: 16) | | fresh/brackish | | | | | |
| | | water, | | | | | |
| | | stomach bug | | | | | |
| Cph | Cronobacter | Host | K4FBD0 | 443 | 42 | −59/+57 | 4 |
| (SEQ ID | phage vB CsaM | member of | | | | | |
| NO: 17) | GAP161 | enterobacteriaceae | | | | | |
| Kph | Klebsiella | Host | D5JF67 | 442 | 44 | −59/+58 | 5 |
| (SEQ ID | phage KP15 | member of | | | | | |
| NO: 18) | | enterobacteriaceae | | | | | |
| SphlME13 | Stenotrophomonas | Host found | J7HXT5 | 438 | 51 | −58/+59 | 7 |
| (SEQ ID | phage | in soil | | | | | |
| NO: 19) | IME13 | | | | | | |
| AphAc42 | Acinetobacter | Host found | E5EYE6 | 442 | 59 | −53/+49 | 9 |
| (SEQ ID | phage Ac42 | in soil | | | | | |
| NO: 20) | | | | | | | |
| SphSP18 | Shigella phage | Host | E3SFA5 | 442 | 59 | −55/+55 | 9 |
| (SEQ ID | SP18 | member of | | | | | |
| NO: 21) | | enterobacteriaceae | | | | | |
| Yph | Yersinia phage | Host | I7J3V8 | 439 | 64 | −52/+52 | 7 |
| (SEQ ID | phiR1-RT | member of | | | | | |
| NO: 22) | | enterobacteriaceae | | | | | |
| SphS16 | Salmonella | Host | M1EA88 | 441 | 72 | −56/+55 | 5 |
| (SEQ ID | phage S16 | member of | | | | | |
| NO: 23) | | enterobacteriaceae | | | | | |
| 1993 | Enterobateria | Host | P32270 | 439 | 100 | −57/+58 | 5 |
| (SEQ ID | phage T4 | member of | | | | | |
| NO: 8) | | enterobacteriaceae | | | | | |

The helicase of the invention preferably comprises a variant of SEQ ID NO: 8 wherein the at least one amino acid which interacts with the sugar and/or base of one or more nucleotides in ssDNA is at least one of H82, N88, P89, F98, D121, V150, P152, F240, F276, S287, H396 and Y415. These numbers correspond to the relevant positions in SEQ ID NO: 8 and may need to be altered in the case of variants where one or more amino acids have been inserted or deleted compared with SEQ ID NO: 8. A skilled person can determine the corresponding positions in a variant as discussed above. The helicase of the invention preferably comprises a variant of SEQ ID NO: 8 wherein the at least one amino acid which interacts with the sugar and/or base of one or more nucleotides in ssDNA is F98 and one or more H82, N88, P89, D121, V150, P152, F240, F276, S287, H396 and Y415, such as F98/H82, F98/N88, F98/P89, F98/D121, F98/V150, F98/P152, F98/F240, F98/F276, F98/S287 or F98/H396.

The helicase of the invention is preferably a variant of SEQ ID NO: 9, 10, 11, 12, 13, 14, 15, 16, 17, 18, 19, 20, 21, 22 or 23 wherein the at least one amino acid which interacts with the sugar and/or base of one or more nucleotides in ssDNA is at least one of the amino acids which correspond to H82, N88, P89, F98, D121, V150, P152, F240, F276, S287, H396 and Y415 in SEQ ID NO: 8. The helicase of the invention preferably comprises a variant of SEQ ID NO: 9, 10, 11, 12, 13, 14, 15, 16, 17, 18, 19, 20, 21, 22 or 23 wherein the at least one amino acid which interacts with the sugar and/or base of one or more nucleotides in ssDNA is the amino acid which corresponds to F98 in SEQ ID NO: 8 and one or more of the amino acids which correspond to H82, N88, P89, D121, V150, P152, F240, F276, S287, H396 and Y415 in SEQ ID NO: 8, such as the amino acids which correspond to F98/H82, F98/N88, F98/P89, F98/D121, F98/V150, F98/P152, F98/F240, F98/F276, F98/S287 or F98/H396.

Table 2 shows the amino acids in SEQ ID NOs: 9 to 23 which correspond to H82, N88, P89, F98, D121, V150, P152, F240, F276, S287, H396 and Y415 in SEQ ID NO: 8.

TABLE 2

| SEQ ID | | | | | | | | | | | | |
|---|---|---|---|---|---|---|---|---|---|---|---|---|
| NO: | | | | | | | | | | | | |
| 8 | H82 | N88 | P89 | F98 | D121 | V150 | P152 | F240 | F276 | S287 | H396 | Y415 |
| 9 | H81 | F87 | D88 | S105 | S131 | V181 | Q183 | R274 | H313 | G314 | H428 | H447 |
| 10 | A144 | Q150 | P151 | G158 | N187 | V217 | K218 | W307 | F344 | K355 | H455 | H473 |
| 11 | H87 | R93 | L94 | G100 | G126 | V154 | N155 | Y240 | Y277 | G280 | H377 | R395 |
| 12 | H112 | K118 | P119 | E128 | G154 | I185 | N187 | Y273 | F309 | K310 | H414 | H433 |
| 13 | H93 | V99 | M100 | D106 | N132 | I159 | Q160 | Y267 | M302 | G303 | H400 | K419 |
| 14 | H74 | H80 | P81 | F90 | D114 | V143 | H145 | Y230 | M266 | P273 | H378 | Y397 |
| 15 | H78 | H84 | P85 | F94 | S117 | E147 | A149 | Y235 | M271 | I279 | H387 | Y406 |
| 16 | H65 | H71 | P72 | F81 | S104 | V133 | H135 | F222 | M258 | I266 | H373 | Y392 |
| 17 | H84 | S90 | P91 | F100 | D126 | V155 | A157 | Y243 | V279 | T290 | H399 | A418 |
| 18 | H84 | S90 | P91 | F100 | D126 | V155 | T157 | Y243 | V279 | V290 | H398 | A417 |
| 19 | Q82 | N88 | P89 | F98 | T121 | V150 | E152 | Y237 | M274 | K285 | H393 | Q412 |
| 20 | H88 | N94 | P95 | F104 | D127 | V156 | P158 | F246 | I282 | S293 | H399 | K418 |
| 21 | H84 | N90 | P91 | F100 | D123 | V152 | P154 | Y242 | M278 | S289 | H399 | M418 |
| 22 | H83 | N89 | P90 | F99 | D122 | V151 | P153 | Y241 | M277 | H288 | H396 | M415 |
| 23 | H83 | N89 | P90 | F99 | D122 | V151 | P153 | F241 | M277 | H288 | H397 | M416 |

The at least one amino acid which interacts with the sugar and/or base of one or more nucleotides in ssDNA is preferably at least one amino acid which intercalates between the nucleotides in ssDNA. Amino acids which intercalate between nucleotides in ssDNA can be modeled as discussed above. The at least one amino acid which intercalates between the nucleotides in ssDNA is preferably at least one of P89, F98 and V150 in SEQ ID NO: 8, such as P89, F98, V150, P89/F98, P89/V150, F98/V150 or P89/F98/V150.

The at least one amino acid which intercalates between the nucleotides in ssDNA in SEQ ID NO: 9, 10, 11, 12, 13, 14, 15, 16, 17, 18, 19, 20, 21, 22 or 23 is preferably at least one of the amino acids which correspond to P89, F98 and V150 in SEQ ID NO: 8, such as P89, F98, V150, P89/F98, P89/V150, F98/V150 or P89/F98/V150. Corresponding amino acids are shown in Table 2 above.

### Larger R Groups

The larger side chain (R group) preferably (a) contains an increased number of carbon atoms, (b) has an increased length, (c) has an increased molecular volume and/or (d) has an increased van der Waals volume. The larger side chain (R group) preferably (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d). Each of (a) to (d) may be measured using standard methods in the art.

The larger side chain (R group) preferably increases the (i) electrostatic interactions (ii) (ii) hydrogen bonding and/or (iii) cation-pi (cation-π) interactions between the at least one amino acid and the one or more nucleotides in ssDNA, such as increases (i); (ii); (iii); (i) and (ii); (i) and (iii); (ii) and (iii); and (i), (ii) and (iii). A skilled person can determine if the R group increases any of these interactions. For instance in (i), positively charged amino acids, such as arginine (R), histidine (H) and lysine (K), have R groups which increase electrostatic interactions. For instance in (ii), amino acids such as asparagine (N), serine (S), glutamine (Q), threonine (T) and histidine (H) have R groups which increase hydrogen bonding. For instance in (iii), aromatic amino acids, such as phenylalanine (F), tryptophan (W), tyrosine (Y) or histidine (H), have R groups which increase cation-pi (cation-π) interactions. Specific substitutions below are labelled (i) to (iii) to reflect these changes. Other possible substitutions are labelled (iv). These (iv) substitutions typically increase the length of the side chain (R group).

The amino acid which comprises a larger side chain (R) may be a non-natural amino acid. The non-natural amino acid may be any of those discussed below.

The amino acid which comprises a larger side chain (R group) is preferably not alanine (A), cysteine (C), glycine (G), selenocysteine (U), methionine (M), aspartic acid (D) or glutamic acid (E).

Histidine (H) is preferably substituted with (i) arginine (R) or lysine (K), (ii) glutamine (Q) or asparagine (N) or (iii) phenylalanine (F), tyrosine (Y) or tryptophan (W). Histidine (H) is more preferably substituted with (a) N, Q or W or (b) Y, F, Q or K.

Asparagine (N) is preferably substituted with (i) arginine (R) or lysine (K), (ii) glutamine (Q) or histidine (H) or (iii) phenylalanine (F), tyrosine (Y) or tryptophan (W). Asparagine (N) is more preferably substituted with R, H, W or Y.

Proline (P) is preferably substituted with (i) arginine (R) or lysine (K), (ii) glutamine (Q), asparagine (N), threonine (T) or histidine (H), (iii) tyrosine (Y), phenylalanine (F) or tryptophan (W) or (iv) leucine (L), valine (V) or isoleucine (I). Proline (P) is more preferably substituted with (i) arginine (R) or lysine (K), (ii) glutamine (Q), asparagine (N), threonine (T) or histidine (H), (iii) phenylalanine (F) or tryptophan (W) or (iv) leucine (L), valine (V) or isoleucine (I). Proline (P) is more preferably substituted with (a) F, (b) L, V, I, T or F or (c) W, F, Y, H, I, L or V.

Valine (V) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L). Valine (V) is more preferably substituted with (i) arginine (R) or lysine (K), (ii) glutamine (Q), asparagine (N) or histidine (H), (iii) tyrosine (Y) or tryptophan (W) or (iv) isoleucine (I) or leucine (L). Valine (V) is more preferably substituted with I or H or I, L, N, W or H.

Phenylalanine (F) is preferably substituted with (i) arginine (R) or lysine (K), (ii) histidine (H) or (iii) tyrosine (Y) or tryptophan (W). Phenylalanine (F) is more preferably substituted with (a) W, (b) W, Y or H, (c) W, R or K or (d) K, H, W or R.

Glutamine (Q) is preferably substituted with (i) arginine (R) or lysine (K) or (iii) phenylalanine (F), tyrosine (Y) or tryptophan (W).

Alanine (A) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L).

Serine (S) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L). Serine (S) is preferably substituted with K, R, W or F Lysine (K) is preferably substituted with (i) arginine (R) or (iii) tyrosine (Y) or tryptophan (W).

Arginine (R) is preferably substituted with (iii) tyrosine (Y) or tryptophan (W).

Methionine (M) is preferably substituted with (i) arginine (R) or lysine (K), (ii) glutamine (Q), asparagine (N) or histidine (H) or (iii) phenylalanine (F), tyrosine (Y) or tryptophan (W).

Leucine (L) is preferably substituted with (i) arginine (R) or lysine (K), (ii) glutamine (Q) or asparagine (N) or (iii) phenylalanine (F), tyrosine (Y) or tryptophan (W).

Aspartic acid (D) is preferably substituted with (i) arginine (R) or lysine (K), (ii) glutamine (Q), asparagine (N) or histidine (H) or (iii) phenylalanine (F), tyrosine (Y) or tryptophan (W). Aspartic acid (D) is more preferably substituted with H, Y or K.

Glutamic acid (E) is preferably substituted with (i) arginine (R) or lysine (K), (ii) glutamine (Q), asparagine (N) or histidine (H) or (iii) phenylalanine (F), tyrosine (Y) or tryptophan (W).

Isoleucine (I) is preferably substituted with (i) arginine (R) or lysine (K), (ii) glutamine (Q), asparagine (N) or histidine (H), (iii) phenylalanine (F), tyrosine (Y) or tryptophan (W) or (iv) leucine (L).

Tyrosine (Y) is preferably substituted with (i) arginine (R) or lysine (K) or (iii) tryptophan (W). Tyrosine (Y) is more preferably substituted with W or R.

The helicase more preferably comprises a variant of SEQ ID NO: 8 and comprises (a) P89F, (b) F98W, (c) V150I, (d) V150H, (e) P89F and F98W, (f) P89F and V150I, (g) P89F and V150H, (h) F98W and V150I, (i) F98W and V150H (j) P89F, F98W and V150I or (k) P89F, F98W and V150H.

The helicase more preferably comprises a variant of SEQ ID NO: 8 which comprises:

| | | |
|---|---|---|
| H82N; | V150W; | S287F; |
| H82Q; | V150H; | H396Y; |
| H82W; | P152W; | H396F; |
| N88R; | P152F; | H396Q; |
| N88H; | P152Y; | H396K; |
| N88W; | P152H; | Y415W; |
| N88Y; | P152I; | Y415R; |
| P89L; | P152L; | F98W/H82N; |
| P89V; | P152V; | F98W/H82Q; |
| P89I; | F240W; | F98W/H82W; |
| P89E; | F240Y; | F98W/N88R; |
| P89T; | F240H; | F98W/N88H; |
| P89F; | F276W; | F98W/N88W; |
| D121H; | F276R; | F98W/N88Y; |
| D121Y; | F276K; | F98W/P89L; |
| D121K; | F276H; | F98W/P89V; |
| V150I; | S287K; | F98W/P89I; |
| V150L; | S287R; | F98W/P89T; |
| V150N; | S287W; | F98W/P89F; |
| F98W/D121H; | F98W/P152Y; | F98W/F276H; |
| F98W/D121Y; | F98W/P152H; | F98W/S287K; |
| F98W/D121K; | F98W/P152I; | F98W/S287R; |
| F98W/V150I; | F98W/P152L; | F98W/S287W; |
| F98W/V150L; | F98W/P152V; | F98W/S287F; |
| F98W/V150N; | F98W/F240W; | F98W/H396Y; |
| F98W/V150W; | F98W/F240Y; | F98W/H396F; |
| F98W/V150H; | F98W/F240H; | F98W/H396Q; |
| F98W/P152W; | F98W/F276W; | F98W/Y415W; |
| F98W/P152F; | F98W/F276R; | or |
| | F98W/F276K; | F98W/Y415R. |

### Phosphate Interactions

The helicase of the invention is preferably one in which at least one amino acid which interacts with one or more phosphate groups in one or more nucleotides in ssDNA is substituted. Any number of amino acids may substituted, such as 1 or more, 2 or more, 3 or more, 4 or more, 5 or more or 6 or more amino acids. Nucleotides in ssDNA each comprise three phosphate groups. Each amino which is substituted may interact with any number of the phosphate groups at a time, such as one, two or three phosphate groups at a time. The amino acids which interact with one or more phosphate groups can be identified using protein modelling as discussed above.

The substitution preferably increases the (i) electrostatic interactions, (ii) hydrogen bonding and/or (iii) cation-pi (cation-π) interactions between the at least one amino acid and the one or more phosphate groups in ssDNA. Preferred substitutions which increase (i), (ii) and (iii) are discussed below using the labelling (i), (ii) and (iii).

The substitution preferably increases the net positive charge of the position. The net charge at any position can be measured using methods known in the art. For instance, the isoelectric point may be used to define the net charge of an amino acid. The net charge is typically measured at about 7.5. The substitution is preferably the substitution of a negatively charged amino acid with a positively charged, uncharged, non-polar or aromatic amino acid. A negatively charged amino acid is an amino acid with a net negative charge. Negatively charged amino acids include, but are not limited to, aspartic acid (D) and glutamic acid (E). A positively charged amino acid is an amino acid with a net positive charge. The positively charged amino acid can be naturally-occurring or non-naturally-occurring. The positively charged amino acid may be synthetic or modified. For instance, modified amino acids with a net positive charge may be specifically designed for use in the invention. A number of different types of modification to amino acids are well known in the art. Preferred naturally-occurring positively charged amino acids include, but are not limited to, histidine (H), lysine (K) and arginine (R).

The uncharged amino acid, non-polar amino acid or aromatic amino acid can be naturally occurring or non-naturally-occurring. It may be synthetic or modified. Uncharged amino acids have no net charge. Suitable uncharged amino acids include, but are not limited to, cysteine (C), serine (S), threonine (T), methionine (M), asparagines (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y).

The helicase preferably comprises a variant of SEQ ID NO: 8 wherein the at least one amino acid which interacts with one or more phosphates in one or more nucleotides in ssDNA is at least one of H64, T80, S83, N242, K243, N293, T394 and K397. These numbers correspond to the relevant positions in SEQ ID NO: 8 and may need to be altered in the case of variants where one or more amino acids have been inserted or deleted compared with SEQ ID NO: 8. A skilled person can determine the corresponding positions in a variant as discussed above.

The helicase preferably comprises a variant of SEQ ID NO: 9, 10, 11, 12, 13, 14, 15, 16, 17, 18, 19, 20, 21, 22 or 23 and wherein the at least one amino acid which interacts with one or more phosphates in one or more nucleotides in ssDNA is at least one of the amino acids which correspond to H64, T80, S83, N242, K243, N293, T394 and K397 in SEQ ID NO: 8.

Table 3 shows the amino acids in SEQ ID NOs: 9 to 23 which correspond to H64, T80, S83, N242, K243, N293, T 394 and K397 in SEQ ID NO: 8.

TABLE 3

| SEQ ID NO: | | | | | | | | |
|---|---|---|---|---|---|---|---|---|
| 8 | H64 | T80 | S83 | N242 | K243 | N293 | T394 | K397 |
| 9 | G63 | T79 | S82 | N276 | A277 | N317 | T426 | K429 |
| 10 | N121 | T142 | K145 | N309 | K310 | N361 | T453 | K456 |
| 11 | H66 | T85 | S88 | N242 | K243 | N286 | T375 | R378 |
| 12 | H89 | T110 | S113 | N275 | K276 | V315 | T412 | K415 |
| 13 | H68 | T91 | S94 | N269 | A270 | N308 | T398 | K401 |
| 14 | H56 | T72 | S75 | N232 | D233 | N279 | T376 | K379 |
| 15 | H60 | T76 | S79 | N237 | K238 | N285 | T385 | K388 |
| 16 | H47 | T63 | S66 | N224 | K225 | N272 | T371 | K374 |
| 17 | H66 | T82 | S85 | N245 | D246 | N296 | T397 | K400 |
| 18 | H66 | T82 | S85 | N245 | A246 | N296 | T396 | K399 |
| 19 | H64 | T80 | S83 | N239 | N240 | N291 | T391 | K394 |
| 20 | H70 | T86 | S89 | N248 | K249 | N299 | T397 | K400 |
| 21 | H66 | T82 | S85 | N244 | K245 | N295 | T397 | K400 |
| 22 | H65 | T81 | S84 | N243 | K244 | N294 | T394 | K397 |
| 23 | H65 | T81 | K84 | N243 | K244 | N294 | T395 | K398 |

Histidine (H) is preferably substituted with (i) arginine (R) or lysine (K), (ii) asparagine (N), serine (S), glutamine (Q) or threonine (T), (iii) phenylalanine (F), tryptophan (W) or tyrosine (Y). Histidine (H) is preferably substituted with (a) N, Q, K or F or (b) N, Q or W.

Threonine (T) is preferably substituted with (i) arginine (R), histidine (H) or lysine (K), (ii) asparagine (N), serine (S), glutamine (Q) or histidine (H) or (iii) phenylalanine (F), tryptophan (W), tyrosine (Y) or histidine (H). Threonine (T) is more preferably substituted with (a) K, Q or N or (b) K, H or N.

Serine (s) is preferably substituted with (i) arginine (R), histidine (H) or lysine (K), (ii) asparagine (N), glutamine (Q), threonine (T) or histidine (H) or (iii) phenylalanine (F), tryptophan (W), tyrosine (Y) or histidine (H). Serine (S) is more preferably substituted with H, N, K, T, R or Q.

Asparagine (N) is preferably substituted with (i) arginine (R), histidine (H) or lysine (K), (ii) serine (S), glutamine (Q), threonine (T) or histidine (H) or (iii) phenylalanine (F), tryptophan (W), tyrosine (Y) or histidine (H). Asparagine (N) is more preferably substituted with (a) H or Q or (b) Q, K or H.

Lysine (K) is preferably substituted with (i) arginine (R) or histidine (H), (ii) asparagine (N), serine (S), glutamine (Q), threonine (T) or histidine (H) or (iii) phenylalanine (F), tryptophan (W), tyrosine (Y) or histidine (H). Lysine (K) is more preferably substituted with (a) Q or H or (b) R, H or Y.

The helicase more preferably comprises a variant of SEQ ID NO: 8 and comprises one or more of, such as all of, (a) H64N, H64Q, H64K or H64F, (b) T80K, T80Q or T80N, (c) S83H, S83N, S83K, S83T, S83R, or S83Q (d) N242H or N242Q, (e) K243Q or K243H, (f) N293Q, N293K or N293H, (g) T394K, T394H or T394N or (h) K397R, K397H or K397Y.

### Combinations

The helicase may be one in which (a) at least one amino acid which interacts with the sugar and/or base of one or more nucleotides in ssDNA is substituted with an amino acid which comprises a larger side chain (R group) and (b) at least one amino acid which interacts with one or more phosphate groups in one or more nucleotides in ssDNA is substituted. The helicase preferably comprises:
(a) a variant of SEQ ID NO: 8 comprising a substitution at F98 as defined above and a substitution at one or more of H64, T80, S83, N242, K243, N293, T394 and K397; or
(b) a variant of SEQ ID NO: 9, 10, 11, 12, 13, 14, 15, 16, 17, 18, 19, 20, 21, 22 or 23 comprising a substitution at the amino acid which corresponds to F98 and a substitution at one of more of the amino acids which correspond to H64, T80, S83, N242, K243, N293, T394 and K397 in SEQ ID NO: 8.

The helicase is preferably a variant of SEQ ID NO: 8 which comprises substitutions at:
F98/H64, such as F98W/H64N, F98W/H64Q, F98W/H64K or F98W/H64F;
F98/T80, such as F98W/T80K, F98W/T80Q, F98W/T80N;
F98/H82, such as F98W/H82N, F98W/H82Q or F98W/H82W;
F98/S83, such as F98W/S83H, F98W/S83N, F98W/S83K, F98W/S83T, F98W/S83R or F98W/S83Q;
F98/N242, such as F98W/N242H, F98W/N242Q, F98W/K243Q or F98W/K243H;
F98/N293, such as F98W/N293Q, F98W/N293K, F98W/N293H, F98W/T394K, F98W/T394H, F98W/T394N, F98W/H396Y, F98W/H396F, F98W/H396Q or F98W/H396K; or
F98/K397, such as F98W/K397R, F98W/K397H or F98W/K397Y.

Preferred combinations in SEQ ID NO: 9, 10, 11, 12, 13, 14, 15, 16, 17, 18, 19, 20, 21, 22 or 23 include the combinations of amino acids which correspond to the combinations in SEQ ID NO: 8 listed above.

### Pore Interaction

The helicase of the invention is further one in which the part of the helicase which interacts with a transmembrane pore comprises one or more modifications, preferably one or more substitutions. The part of the helicase which interacts with a transmembrane pore is typically the part of the helicase which interacts with a transmembrane pore when the helicase is used to control the movement of a polynucleotide through the pore, for instance as discussed in more detail below. The part typically comprises the amino acids that interact with or contact the pore when the helicase is used to control the movement of a polynucleotide through the pore, for instance as discussed in more detail below. The part typically comprises the amino acids that interact with or contact the pore when the helicase is bound to or attached to a polynucleotide which is moving through the pore under an applied potential.

In SEQ ID NO: 8, the part which interacts with the transmembrane pore typically comprises the amino acids at positions 1, 2, 3, 4, 5, 6, 51, 176, 177, 178, 179, 180, 181, 185, 189, 191, 193, 194, 195, 197, 198, 199, 200, 201, 202, 203, 204, 207, 208, 209, 210, 211, 212, 213, 216, 219, 220, 221, 223, 224, 226, 227, 228, 229, 247, 254, 255, 256, 257, 258, 259, 260, 261, 298, 300, 304, 308, 318, 319, 321, 337, 347, 350, 351, 405, 415, 422, 434, 437, 438. These numbers correspond to the relevant positions in SEQ ID NO: 8 and may need to be altered in the case of variants where one or more amino acids have been inserted or deleted compared with SEQ ID NO: 8. A skilled person can determine the corresponding positions in a variant as discussed above. The part which interacts with the transmembrane pore preferably comprises the amino acids at
(a) positions 1, 2, 4, 51, 177, 178, 179, 180, 185, 193, 195, 197, 198, 199, 200, 202, 203, 204, 207, 208, 209, 210, 211, 212, 216, 221, 223, 224, 226, 227, 228, 229, 254, 255, 256, 257, 258, 260, 304, 318, 321, 347, 350, 351, 405, 415, 422, 434, 437 and 438 in SEQ ID NO: 8; or
(b) positions 1, 2, 178, 179, 180, 185, 195, 197, 198, 199, 200, 202, 203, 207, 209, 210, 212, 216, 221, 223, 226, 227, 255, 258, 260, 304, 350 and 438 in SEQ ID NO: 8.

The part which interacts with the transmembrane pore preferably comprises one or more of, such as 2, 3, 4 or 5 of, the amino acids at positions K194, W195, K198, K199 and E258 in SEQ ID NO: 8. The variant of SEQ ID NO: 8 preferably comprises a modification at one or more of (a), K194, (b) W195, (c) D198, (d) K199 and (d) E258. The variant of SEQ ID NO: 8 preferably comprises a substitution at one or more of (a) K194, such as K194L, (b) W195, such as W195A, (c) D198, such as D198V, (d) K199, such as K199L and (d) E258, such as E258L. The variant may comprise {a}; {b}; {c}; {d}; {e}; {a,b}; {a,c}; {a,d}; {a,e}; {b,c}; {b,d}; {b,e}; {c,d}; {c,e}; {d,e}; {a,b,c}; {a,b,d}; {a,b,e}; {a,c,d}; {a,c,e}; {a,d,e}; {b,c,d}; {b,c,e}; {b,d,e}; {c,d,e}; {a,b,c,d}; {a,b,c,e}; {a,b,d,e}; {a,c,d,e}; {b,c,d,e}; or {a,b,c,d,e}. The modifications or substitutions set out in this paragraph are preferred when the modified polynucleotide binding protein interacts with a pore derived from MspA, particularly any of the modified pores discussed below.

The part of the polynucleotide binding protein which interacts with the transmembrane pore preferably comprises the amino acid at position 194 or 199 of SEQ ID NO: 8. The variant preferably comprises K194A, K194V, K194F, K194D, K194S, K194W or K194L and/or K199A, K199V, K199F, K199D, K199S, K199W or K199L. In SEQ ID NO: 9, 10, 11, 12, 13, 14, 15, 16, 17, 18, 19, 20, 21, 22 or 23, the part which interacts with the transmembrane pore typically comprises the amino acids at positions which correspond to those in SEQ ID NO: 8 listed above. Amino acids in SEQ ID NOs: 9, 10, 11, 12, 13, 14, 15, 16, 17, 18, 19, 20, 21, 22 and 23 which correspond to these positions in SEQ ID NO: 9 can be identified using the alignment shown below.

| | | | | | |
|---|---|---|---|---|---|
| SEQ ID NO: 8 | K194 | W195 | D198 | K199 | E258 |
| SEQ ID NO: 9 | L230 | E231 | H234 | Y235 | R293 |
| SEQ ID NO: 10 | W259 | N260 | T263 | Y264 | E326 |
| SEQ ID NO: 11 | A192 | D193 | F196 | G197 | A259 |
| SEQ ID NO: 12 | I224 | K225 | D228 | F229 | Q292 |
| SEQ ID NO: 13 | Q213 | D214 | Y217 | A218 | A286 |
| SEQ ID NO: 14 | Q185 | W186 | T189 | N190 | N248 |
| SEQ ID NO: 15 | G190 | W191 | P194 | N195 | K253 |
| SEQ ID NO: 16 | G177 | W178 | Q181 | N182 | K240 |
| SEQ ID NO: 17 | K200 | M201 | P204 | M205 | K261 |
| SEQ ID NO: 18 | K200 | P201 | P204 | L205 | K261 |
| SEQ ID NO: 19 | K193 | W194 | E197 | K198 | A256 |
| SEQ ID NO: 20 | N200 | W201 | E204 | N205 | N264 |
| SEQ ID NO: 21 | G196 | W197 | D200 | C201 | E260 |
| SEQ ID NO: 22 | G195 | W196 | E199 | N200 | E259 |
| SEQ ID NO: 23 | S195 | W196 | E199 | K200 | Q259 |

### Preferred Combinations

The helicase of the invention preferably comprises a variant of SEQ ID NO: 8 which comprises a substitution at F98, such as F98R, F98K, F98Q, F98N, F98H, F98Y, F98F or F98W, and a substitution at K194, such as K194A, K194V, K194F, K194D, K194S, K194W or K194L, and/or K199, such as K199A, K199V, K199F, K199D, K199S, K199W or K199L. The helicase of the invention preferably comprises a variant of SEQ ID NO: 9, 10, 11, 12, 13, 14, 15, 16, 17, 18, 19, 20, 21, 22 or 23 which comprises a substitution at the position which corresponds to F98 in SEQ ID NO: 8 and a substitution at the position(s) which correspond to K194 and/or K199 in SEQ ID NO: 8. These corresponding positions may be replaced with any of the amino acids listed above for F98, K194 and K119 in SEQ ID NO: 8.

The helicase is preferably a variant of SEQ ID NO: 8 which comprises substitutions at:
F98/K194/H64, such as F98W/K194L/H64N, F98W/K194L/H64Q, F98W/K194L/H64K or F98W/K194L/H64F;
F98/K194/T80, such as F98W/K194L/T80K, F98W/K194L/T80Q or F98W/K194L/T80N;
F98/K194/H82, such as F98W/K194L/H82N, F98W/K194L/H82Q or F98W/K194L/H82W
F98/S83/K194, such as F98W/S83H/K194L, F98W/S83T/K194L, F98W/S83R/K194L, F98W/S83Q/K194L, F98W/S83N/K194L, F98W/S83K/K194L, F98W/N88R/K194L, F98W/N88H/K194L, F98W/N88W/K194L or F98W/N88Y/K194L;
F98/S83/K194/F276, such as F98W/S83H/K194L/F276K;
F98/P89/K194, such as F98W/P89L/K194L, F98W/P89V/K194L, F98W/P891/K194L or F98W/P89T/K194L;
F98/D121/K194, such as F98W/D121H/K194L, F98W/D121Y/K194L or F98W/D121K/K194L;
F98/V150/K194, such as F98W/V150I/K194L, F98W/V150L/K194L, F98W/V150N/K194L, F98W/V150W/K194L or F98W/V150H/K194L;
F98/P152/K194, such as F98W/P152W/K194L, F98W/P152F/K194L, F98W/P152Y/K194L, F98W/P152H/K194L, F98W/P1521/K194L, F98W/P152L/K194L or F98W/P152V/K194L;
F98/F240/K194, such as F98W/F240W/K194L, F98W/F240Y/K194L or F98W/F240H/K194L;
F98/N242/K194, such as F98W/N242H/K194L or F98W/N242Q/K194L;
F98/K194/F276, such as F98W/K194L/F276K, F98W/K194L/F276H, F98W/K194L/F276W or F98W/K194L/F276R;
F98/K194/S287, such as F98W/K194L/S287K, F98W/K194L/S287R, F98W/K194L/S287W or F98W/K194L/S287F;
F98/N293/K194, such as F98W/N293Q/K194L, F98W/N293K/K194L or F98W/N293H/K194L;
F98/T394/K194, such as F98W/T394K/K194L, F98W/T394H/K194L or F98W/T394N/K194L;
F98/H396/K194, such as F98W/H396Y/K194L, F98W/H396F/K194L, F98W/H396Q/K194L or F98W/H396K/K194L;
F98/K397/K194, such as F98W/K397R/K194L, F98W/K397H/K194L or F98W/K397Y/K194L; or
F98/Y415/K194, such as F98W/Y415W/K194L or F98W/Y415R/K194L.

In any of the above combinations, K194 may be replaced with any of W195, D198, K199 and E258.

The helicase is preferably a variant of SEQ ID NO: 9, 10, 11, 12, 13, 14, 15, 16, 17, 18, 19, 20, 21, 22 or 23 which comprises substitutions at amino acids which correspond to the combinations in SEQ ID NO: 8 listed above.

Modifications in the Tower Domain and/or Pin Domain and/or 1A Domain

The helicase of the invention is preferably one in which at least one cysteine residue (i.e. one or more cysteine residues) and/or at least one non-natural amino acid (i.e. one or more non-natural amino acids) have been introduced into (i) the tower domain and/or (ii) the pin domain and/or the (iii) 1A (RecA-like motor) domain, wherein the helicase has the ability to control the movement of a polynucleotide. These types of modification are disclosed in PCT/GB2014/052736 (WO 2015/055981). At least one cysteine residue and/or at least one non-natural amino acid may be introduced into the tower domain, the pin domain, the 1A domain, the tower domain and the pin domain, the tower domain and the 1A domain or the tower domain, the pin domain and the 1A domain.

The helicase of the invention is preferably one in which at least one cysteine residue and/or at least one non-natural amino acid have been introduced into each of (i) the tower domain and (ii) the pin domain and/or the 1A (RecA-like motor) domain, i.e. into the tower domain and the pin domain, the tower domain and the 1A domain or the tower domain, the pin domain and the 1A domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are/is preferably introduced by substitution. Methods for doing this are known in the art.

These modifications do not prevent the helicase from binding to a polynucleotide. These modifications decrease the ability of the polynucleotide to unbind or disengage from the helicase. In other words, the one or more modifications increase the processivity of the helicase by preventing dissociation from the polynucleotide strand. The thermal stability of the enzyme is typically also increased by the one or more modifications giving it an improved structural stability that is beneficial in Strand Sequencing.

A non-natural amino acid is an amino that is not naturally found in a helicase. The non-natural amino acid is preferably not histidine, alanine, isoleucine, arginine, leucine, asparagine, lysine, aspartic acid, methionine, cysteine, phenylalanine, glutamic acid, threonine, glutamine, tryptophan, glycine, valine, proline, serine or tyrosine. The non-natural amino acid is more preferably not any of the twenty amino acids in the previous sentence or selenocysteine.

Preferred non-natural amino acids for use in the invention include, but are not limited, to 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O-2-Propyn-1-yl-L-tyrosine, 4-(Dihydroxyboryl)-L-phenylalanine, 4-[(Ethylsulfanyl)carbonyl]-L-phenylalanine, (2S)-2-amino-3-4-[(propan-2-ylsulfanyl)carbonyl]phenyl;propanoic acid, (2S)-2-amino-3-4-[(2-amino-3-sulfanylpropanoyl)amino]phenyl;propanoic acid, O-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, O-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2,2′-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, 0-(4,5-Dimethoxy-2-nitrobenzyl)-L-serine, (2S)-2-amino-6-([(2-nitrobenzyl)oxy]carbonyl;amino)hexanoic acid, 0-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-1-naphthyl]sulfonylamino]propanoic acid, (2S)-2-amino-4-(7-hydroxy-2-oxo-2H-chromen-4-yl)butanoic acid, (2S)-3-[(6-acetylnaphthalen-2-yl)amino]-2-aminopropanoic acid, 4-(Carboxymethyl)phenylalanine, 3-Nitro-L-tyrosine, O-Sulfo-L-tyrosine, (2R)-6-Acetamido-2-ammoniohexanoate, 1-Methylhistidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N⁶-[(2R,3R)-3-Methyl-3,4-dihydro-2H-pyrrol-2-yl]carbonyl;-L-lysine, N⁶-[(Benzyloxy)carbonyl]lysine, (2S)-2-amino-6-[(cyclopentylcarbonyl)amino]hexanoic acid, N⁶-[(Cyclopentyloxy)carbonyl]-L-lysine, (2S)-2-amino-6-[(2R)-tetrahydrofuran-2-ylcarbonyl]amino;hexanoic acid, (2S)-2-amino-8-[(2R,3S)-3-ethynyltetrahydrofuran-2-yl]-8-oxooctanoic acid, N⁶-(tert-Butoxycarbonyl)-L-lysine, (2S)-2-Hydroxy-6-([(2-methyl-2-propanyl)oxy]carbonyl;amino)hexanoic acid, N⁶-[(Allyloxy)carbonyl]lysine, (2S)-2-amino-6-([(2-azidobenzyl)oxy]carbonyl;amino)hexanoic acid, N⁶-L-Prolyl-L-lysine, (2S)-2-amino-6-[(prop-2-yn-1-yloxy)carbonyl]amino;hexanoic acid and N⁶-[(2-Azidoethoxy)carbonyl]-L-lysine. The most preferred non-natural amino acid is 4-azido-L-phenylalanine (Faz).

Table 4 below (which is separated in two parts) identifies the residues making up each domain in each Dda homologue (SEQ ID NOs: 8 to 23).

TABLE 4

| Homologue | SEQ ID NO | 1A | 2A |
|---|---|---|---|
| Dda-Rma-DSM | 9 | M1-I84 + R113-Y211 | R212-E294 + G422-S678 |
| Dda-Csp | 10 | M1-L147 + S166-V240 | R241-N327 + A449-G496 |
| Dda-Sru | 11 | M1-L90 + E108-H173 | R174-D260 + A371-V421 |
| Dda-Sgo | 12 | M1-L115 + N136-V205 | R206-K293 + I408-L500 |
| Dda-Vph12B8 | 13 | M1-L96 + F114-V194 | R195-D287 + V394-Q450 |
| Dda-Vph | 14 | M1-L77 + V96-V166 | R167-T249 + L372-N421 |
| Dda-Aph65 | 15 | M1-M81 + L99-M171 | R172-T254 + L381-K434 |
| Dda-AphCC2 | 16 | M1-M68 + M86-M158 | R159-T241 + L367-K420 |
| Dda-Cph | 17 | M1-L87 + A108-M181 | R182-T262 + L393-V443 |
| Dda-Kph | 18 | M1-L87 + A108-M181 | R182-T262 + L392-V442 |
| Dda-SphIME13 | 19 | M1-L85 + T103-K176 | R177-N257 + L387-V438 |
| Dda-AphAc42 | 20 | M1-L91 + V109-M183 | R184-T265 + L393-I442 |
| Dda-SphSP18 | 21 | M1-L87 + M105-M179 | R180-T261 + L393-V442 |
| Dda-Yph | 22 | M1-L86 + V104-K178 | R179-T260 + L390-I439 |
| Dda-SphS16 | 23 | M1-L86 + V104-M178 | R179-T260 + L391-V441 |
| Dda-1993 | 8 | M1-L85 + V103-K177 | R178-T259 + L390-V439 |

| Homologue | SEQ ID | tower | pin | hook |
|---|---|---|---|---|
| Dda-Rma-DSM | 9 | G295-N309 + F316-Y421 | Y85-L112 | A310-L315 |
| Dda-Csp | 10 | V328-P342 + N360-Y448 | K148-N165 | V343-L359 |
| Dda-Sru | 11 | A261-T275 + T285-Y370 | G91-E107 | W276-L284 |
| Dda-Sgo | 12 | G294-I307 + T314-Y407 | G116-T135 | R308-Y313 |
| Dda-Vph12B8 | 13 | V288-E301 + N307-N393 | G97-P113 | M302-W306 |
| Dda-Vph | 14 | S250-P264 + E278-S371 | K78-E95 | V265-I277 |
| Dda-Aph65 | 15 | K255-P269 + T284-S380 | K82-K98 | V270-F283 |
| Dda-AphCC2 | 16 | D242-P256 + T271-S366 | K69-K85 | V257-F270 |
| Dda-Cph | 17 | T263-P277 + N295-P392 | K88-K107 | L278-Y294 |
| Dda-Kph | 18 | D263-P277 + N295-A391 | K88-K107 | L278-Y294 |
| Dda-SphIME13 | 19 | A258-P272 + N290-P386 | K86-G102 | L273-F289 |
| Dda-AphAc42 | 20 | L266-P280 + N298-A392 | K92-D108 | L281-F297 |
| Dda-SphSP18 | 21 | D262-P276 + N294-A392 | K88-E104 | H277-F293 |
| Dda-Yph | 22 | D261-P275 + N293-A389 | K87-E103 | L276-F292 |
| Dda-SphS16 | 23 | E261-P275 + T293-A390 | K87-E103 | L276-F292 |
| Dda-1993 | 8 | D260-P274 + N292-A389 | K86-E102 | L275-F291 |

The helicase of the invention preferably comprises a variant of SEQ ID NO: 8 in which at least one cysteine residue and/or at least one non-natural amino acid have been introduced into (i) the tower domain (residues D260-P274 and N292-A389) and/or (ii) the pin domain (residues K86-E102) and/or the (iii) 1A domain (residues M1-L85 and V103-K177). The at least one cysteine residue and/or at least one non-natural amino acid are preferably introduced into residues N292-A389 of the tower domain.

The helicase of the invention preferably comprises a variant of SEQ ID NO: 9 in which at least one cysteine residue and/or at least one non-natural amino acid have been introduced into (i) the tower domain (residues G295-N309 and F316-Y421) and/or (ii) the pin domain (residues Y85-L112) and/or the (iii) 1A domain (residues M1-I84 and R113-Y211). The at least one cysteine residue and/or at least one non-natural amino acid are preferably introduced into residues F316-Y421 of the tower domain.

The helicase of the invention preferably comprises a variant of SEQ ID NO: 10 in which at least one cysteine residue and/or at least one non-natural amino acid have been introduced into (i) the tower domain (residues V328-P342 and N360-Y448) and/or (ii) the pin domain (residues K148-N165) and/or the (iii) 1A domain (residues M1-L147 and 5166-V240). The at least one cysteine residue and/or at least one non-natural amino acid are preferably introduced into residues N360-Y448 of the tower domain.

The helicase of the invention preferably comprises a variant of SEQ ID NO: 11 in which at least one cysteine residue and/or at least one non-natural amino acid have been introduced into (i) the tower domain (residues A261-T275 and T285-Y370) and/or (ii) the pin domain (residues G91-E107) and/or the (iii) 1A domain (residues M1-L90 and E108-H173). The at least one cysteine residue and/or at least one non-natural amino acid are preferably introduced into residues T285-Y370 of the tower domain.

The helicase of the invention preferably comprises a variant of SEQ ID NO: 12 in which at least one cysteine residue and/or at least one non-natural amino acid have been introduced into (i) the tower domain (residues G294-I307 and T314-Y407) and/or (ii) the pin domain (residues G116-T135) and/or the (iii) 1A domain (residues M1-L115 and N136-V205). The at least one cysteine residue and/or at least one non-natural amino acid are preferably introduced into residues T314-Y407 of the tower domain.

The helicase of the invention preferably comprises a variant of SEQ ID NO: 13 in which at least one cysteine residue and/or at least one non-natural amino acid have been introduced into (i) the tower domain (residues V288-E301 and N307-N393) and/or (ii) the pin domain (residues G97-P113) and/or the (iii) 1A domain (residues M1-L96 and F114-V194). The at least one cysteine residue and/or at least one non-natural amino acid are preferably introduced into residues N307-N393 of the tower domain.

The helicase of the invention preferably comprises a variant of SEQ ID NO: 14 in which at least one cysteine residue and/or at least one non-natural amino acid have been introduced into (i) the tower domain (residues 5250-P264 and E278-S371) and/or (ii) the pin domain (residues K78-E95) and/or the (iii) 1A domain (residues M1-L77 and V96-V166). The at least one cysteine residue and/or at least one non-natural amino acid are preferably introduced into residues E278-S371 of the tower domain.

The helicase of the invention preferably comprises a variant of SEQ ID NO: 15 in which at least one cysteine residue and/or at least one non-natural amino acid have been introduced into (i) the tower domain (residues K255-P269 and T284-S380) and/or (ii) the pin domain (residues K82-K98) and/or the (iii) 1A domain (residues M1-M81 and L99-M171). The at least one cysteine residue and/or at least one non-natural amino acid are preferably introduced into residues T284-S380 of the tower domain.

The helicase of the invention preferably comprises a variant of SEQ ID NO: 16 in which at least one cysteine residue and/or at least one non-natural amino acid have been introduced into (i) the tower domain (residues D242-P256 and T271-S366) and/or (ii) the pin domain (residues K69-K85) and/or the (iii) 1A domain (residues M1-M68 and M86-M158). The at least one cysteine residue and/or at least one non-natural amino acid are preferably introduced into residues T271-S366 of the tower domain.

The helicase of the invention preferably comprises a variant of SEQ ID NO: 17 in which at least one cysteine residue and/or at least one non-natural amino acid have been introduced into (i) the tower domain (residues T263-P277 and N295-P392) and/or (ii) the pin domain (residues K88-K107) and/or the (iii) 1A domain (residues M1-L87 and A108-M181). The at least one cysteine residue and/or at least one non-natural amino acid are preferably introduced into residues N295-P392 of the tower domain.

The helicase of the invention preferably comprises a variant of SEQ ID NO: 18 in which at least one cysteine residue and/or at least one non-natural amino acid have been introduced into (i) the tower domain (residues D263-P277 and N295-A391) and/or (ii) the pin domain (residues K88-K107) and/or the (iii) 1A domain (residues M1-L87 and A108-M181). The at least one cysteine residue and/or at least one non-natural amino acid are preferably introduced into residues N295-A391 of the tower domain.

The helicase of the invention preferably comprises a variant of SEQ ID NO: 19 in which at least one cysteine residue and/or at least one non-natural amino acid have been introduced into (i) the tower domain (residues A258-P272 and N290-P386) and/or (ii) the pin domain (residues K86-G102) and/or the (iii) 1A domain (residues M1-L85 and T103-K176). The at least one cysteine residue and/or at least one non-natural amino acid are preferably introduced into residues N290-P386 of the tower domain.

The helicase of the invention preferably comprises a variant of SEQ ID NO: 20 in which at least one cysteine residue and/or at least one non-natural amino acid have been introduced into (i) the tower domain (residues L266-P280 and N298-A392) and/or (ii) the pin domain (residues K92-D108) and/or the (iii) 1A domain (residues M1-L91 and V109-M183). The at least one cysteine residue and/or at least one non-natural amino acid are preferably introduced into residues N298-A392 of the tower domain.

The helicase of the invention preferably comprises a variant of SEQ ID NO: 21 in which at least one cysteine residue and/or at least one non-natural amino acid have been introduced into (i) the tower domain (residues D262-P276 and N294-A392) and/or (ii) the pin domain (residues K88-E104) and/or the (iii) 1A domain (residues M1-L87 and M105-M179). The at least one cysteine residue and/or at least one non-natural amino acid are preferably introduced into residues N294-A392 of the tower domain.

The helicase of the invention preferably comprises a variant of SEQ ID NO: 22 in which at least one cysteine residue and/or at least one non-natural amino acid have been introduced into (i) the tower domain (residues D261-P275 and N293-A389) and/or (ii) the pin domain (residues K87-E103) and/or the (iii) 1A domain (residues M1-L86 and V104-K178). The at least one cysteine residue and/or at least one non-natural amino acid are preferably introduced into residues N293-A389 of the tower domain.

The helicase of the invention preferably comprises a variant of SEQ ID NO: 23 in which at least one cysteine residue and/or at least one non-natural amino acid have been introduced into (i) the tower domain (residues E261-P275 and T293-A390) and/or (ii) the pin domain (residues K87-E103) and/or the (iii) 1A domain (residues M1-L86 and V104-M178). The at least one cysteine residue and/or at least one non-natural amino acid are preferably introduced into residues T293-A390 of the tower domain.

The helicase of the invention preferably comprises a variant of any one of SEQ ID NOs: 8 to 23 in which at least one cysteine residue and/or at least one non-natural amino acid have been introduced into each of (i) the tower domain and (ii) the pin domain and/or the 1A domain. The helicase of the invention more preferably comprises a variant of any one of SEQ ID NOs: 8 to 23 in which at least one cysteine residue and/or at least one non-natural amino acid have been introduced into each of (i) the tower domain, (ii) the pin domain and (iii) the 1A domain. Any number and combination of cysteine residues and non-natural amino acids may be introduced as discussed above.

The helicase of the invention preferably comprises a variant of SEQ ID NO: 8 which comprises (i) E94C and/or A360C; (ii) E93C and/or K358C; (iii) E93C and/or A360C; (iv) E93C and/or E361C; (v) E93C and/or K364C; (vi) E94C and/or L354C; (vii) E94C and/or K358C; (viii) E93C and/or L354C; (ix) E94C and/or E361C; (x) E94C and/or K364C; (xi) L97C and/or L354C; (xii) L97C and/or K358C; (xiii) L97C and/or A360C; (xiv) L97C and/or E361C; (xv) L97C and/or K364C; (xvi) K123C and/or L354C; (xvii) K123C and/or K358C; (xviii) K123C and/or A360C; (xix) K123C and/or E361C; (xx) K123C and/or K364C; (xxi) N155C and/or L354C; (xxii) N155C and/or K358C; (xxiii) N155C and/or A360C; (xxiv) N155C and/or E361C; (xxv) N155C and/or K364C; (xxvi) any of (i) to (xxv) and G357C; (xxvii) any of (i) to (xxv) and Q100C; (xxviii) any of (i) to (xxv) and I127C; (xxix) any of (i) to (xxv) and Q100C and I127C; (xxx) E94C and/or F377C; (xxxi) N95C; (xxxii) T91C; (xxxiii) Y92L, E94Y, Y350N, A360C and Y363N; (xxxiv) E94Y and A360C; (xxxv) A360C; (xxxvi) Y92L, E94C, Y350N, A360Y and Y363N; (xxxvii) Y92L, E94C and A360Y; (xxxviii) E94C and/or A360C and F276A; (xxxix) E94C and/or L356C; (xl) E93C and/or E356C; (xli) E93C and/or G357C; (xlii) E93C and/or A360C; (xliii) N95C and/or W378C; (xliv) T91C and/or S382C; (xlv) T91C and/or W378C; (xlvi) E93C and/or N353C; (xlvii) E93C and/or S382C; (xlviii) E93C and/or K381C; (xlix) E93C and/or D379C; (1) E93C and/or S375C; (li) E93C and/or W378C; (lii) E93C and/or W374C; (liii) E94C and/or N353C; (liv) E94C and/or S382C; (lv) E94C and/or K381C; (lvi) E94C and/or D379C; (lvii) E94C and/or S375C; (lviii) E94C and/or W378C; (lix) E94C and/or W374C; (lx) E94C and A360Y; (lxi) E94C, G357C and A360C or (lxii) T2C, E94C and A360C. In any one of (i) to (lxii), and/or is preferably and.

The helicase of the invention preferably comprises a variant of any one of SEQ ID NOs: 9 to 23 which comprises a cysteine residue at the positions which correspond to those in SEQ ID NO: 8 as defined in any of (i) to (lxii). Positions in any one of SEQ ID NOs: 9 to 23 which correspond to those in SEQ ID NO: 8 can be identified using the alignment of SEQ ID NOs: 8 to 23 below. The helicase of the invention preferably comprises a variant of SEQ ID NO: 11 which comprises (a) D99C and/or L341C, (b) Q98C and/or L341C or (d) Q98C and/or A340C. The helicase of the invention preferably comprises a variant of SEQ ID NO: 15 which comprises D90C and/or A349C. The helicase of the invention preferably comprises a variant of SEQ ID NO: 21 which comprises D96C and/or A362C.

The helicase of the invention preferably comprises a variant of any one of SEQ ID NOs: 8 to 23 as defined in any one of (i) to (lxii) in which Faz is introduced at one or more of the specific positions instead of cysteine. Faz may be introduced at each specific position instead of cysteine. The helicase of the invention preferably comprises a variant of SEQ ID NO: 8 which comprises (i) E94Faz and/or A360C; (ii) E94C and/or A360Faz; (iii) E94Faz and/or A360Faz; (iv) Y92L, E94Y, Y350N, A360Faz and Y363N; (v) A360Faz; (vi) E94Y and A360Faz; (vii) Y92L, E94Faz, Y350N, A360Y and Y363N; (viii) Y92L, E94Faz and A360Y; (ix) E94Faz and A360Y; and (x) E94C, G357Faz and A360C.

The helicase of the invention preferably further comprises one or more single amino acid deletions from the pin domain. Any number of single amino acid deletions may be made, such as 1, 2, 3, 4, 5, 6, 7, 8, 9, 10 or more. The helicase more preferably comprises a variant of SEQ ID NO: 8 which comprises deletion of E93, deletion of E95 or deletion of E93 and E95. The helicase more preferably comprises a variant of SEQ ID NO: 8 which comprises (a) E94C, deletion of N95 and A360C; (b) deletion of E93, deletion of E94, deletion of N95 and A360C; (c) deletion of E93, E94C, deletion of N95 and A360C or (d) E93C, deletion of N95 and A360C. The helicase of the invention preferably comprises a variant of any one of SEQ ID NOs: 9 to 23 which comprises deletion of the position corresponding to E93 in SEQ ID NO: 8, deletion of the position corresponding to E95 in SEQ ID NO: 8 or deletion of the positions corresponding to E93 and E95 in SEQ ID NO: 8.

The helicase of the invention preferably further comprises one or more single amino acid deletions from the hook domain. Any number of single amino acid deletions may be made, such as 1, 2, 3, 4, 5, 6, 7, 8, 9, 10 or more. The helicase more preferably comprises a variant of SEQ ID NO: 8 which comprises deletion of any number of positions T278 to S287. The helicase more preferably comprises a variant of SEQ ID NO: 8 which comprises (a) E94C, deletion of Y279 to K284 and A360C, (b) E94C, deletion of T278, Y279, V286 and S287 and A360C, (c) E94C, deletion of 1281 and K284 and replacement with a single G and A360C, (d) E94C, deletion of K280 and P2845 and replacement with a single G and A360C, or (e) deletion of Y279 to K284, E94C, F276A and A230C. The helicase of the invention preferably comprises a variant of any one of SEQ ID NOs: 9 to 23 which comprises deletion of any number of the positions corresponding to 278 to 287 in SEQ ID NO: 8.

The helicase of the invention preferably further comprises one or more single amino acid deletions from the pin domain and one or more single amino acid deletions from the hook domain.

The helicase of the invention is preferably one in which at least one cysteine residue and/or at least one non-natural amino acid have been further introduced into the hook domain and/or the 2A (RecA-like) domain. Any number and combination of cysteine residues and non-natural amino acids may be introduced as discussed above for the tower, pin and 1A domains.

The helicase of the invention preferably comprises a variant of SEQ ID NO: 8 in which at least one cysteine residue and/or at least one non-natural amino acid have further been introduced into the hook domain (residues L275-F291) and/or the 2A (RecA-like) domain (residues R178-T259 and L390-V439).

The helicase of the invention preferably comprises a variant of SEQ ID NO: 9 in which at least one cysteine residue and/or at least one non-natural amino acid have further been introduced into the hook domain (residues A310-L315) and/or the 2A (RecA-like) domain (residues R212-E294 and G422-S678).

The helicase of the invention preferably comprises a variant of SEQ ID NO: 10 in which at least one cysteine residue and/or at least one non-natural amino acid have further been introduced into the hook domain (residues V343-L359) and/or the 2A (RecA-like) domain (residues R241-N327 and A449-G496).

The helicase of the invention preferably comprises a variant of SEQ ID NO: 11 in which at least one cysteine residue and/or at least one non-natural amino acid have further been introduced into the hook domain (residues W276-L284) and/or the 2A (RecA-like) domain (residues R174-D260 and A371-V421).

The helicase of the invention preferably comprises a variant of SEQ ID NO: 12 in which at least one cysteine residue and/or at least one non-natural amino acid have further been introduced into the hook domain (residues R308-Y313) and/or the 2A (RecA-like) domain (residues R206-K293 and I408-L500).

The helicase of the invention preferably comprises a variant of SEQ ID NO: 13 in which at least one cysteine residue and/or at least one non-natural amino acid have further been introduced into the hook domain (residues M302-W306) and/or the 2A (RecA-like) domain (residues R195-D287 and V394-Q450).

The helicase of the invention preferably comprises a variant of SEQ ID NO: 14 in which at least one cysteine residue and/or at least one non-natural amino acid have further been introduced into the hook domain (residues V265-I277) and/or the 2A (RecA-like) domain (residues R167-T249 and L372-N421).

The helicase of the invention preferably comprises a variant of SEQ ID NO: 15 in which at least one cysteine residue and/or at least one non-natural amino acid have further been introduced into the hook domain (residues V270-F283) and/or the 2A (RecA-like) domain (residues R172-T254 and L381-K434).

The helicase of the invention preferably comprises a variant of SEQ ID NO: 16 in which at least one cysteine residue and/or at least one non-natural amino acid have further been introduced into the hook domain (residues V257-F270) and/or the 2A (RecA-like) domain (residues R159-T241 and L367-K420).

The helicase of the invention preferably comprises a variant of SEQ ID NO: 17 in which at least one cysteine residue and/or at least one non-natural amino acid have further been introduced into the hook domain (residues L278-Y294) and/or the 2A (RecA-like) domain (residues R182-T262 and L393-V443).

The helicase of the invention preferably comprises a variant of SEQ ID NO: 18 in which at least one cysteine residue and/or at least one non-natural amino acid have further been introduced into the hook domain (residues L278-Y294) and/or the 2A (RecA-like) domain (residues R182-T262 and L392-V442).

The helicase of the invention preferably comprises a variant of SEQ ID NO: 19 in which at least one cysteine residue and/or at least one non-natural amino acid have further been introduced into the hook domain (residues L273-F289) and/or the 2A (RecA-like) domain (residues R177-N257 and L387-V438).

The helicase of the invention preferably comprises a variant of SEQ ID NO: 20 in which at least one cysteine residue and/or at least one non-natural amino acid have further been introduced into the hook domain (residues L281-F297) and/or the 2A (RecA-like) domain (residues R184-T265 and L393-I442).

The helicase of the invention preferably comprises a variant of SEQ ID NO: 21 in which at least one cysteine residue and/or at least one non-natural amino acid have further been introduced into the hook domain (residues H277-F293) and/or the 2A (RecA-like) domain (residues R180-T261 and L393-V442).

The helicase of the invention preferably comprises a variant of SEQ ID NO: 22 in which at least one cysteine residue and/or at least one non-natural amino acid have further been introduced into the hook domain (residues L276-F292) and/or the 2A (RecA-like) domain (residues R179-T260 and L390-I439).

The helicase of the invention preferably comprises a variant of SEQ ID NO: 23 in which at least one cysteine residue and/or at least one non-natural amino acid have further been introduced into the hook domain (residues L276-F292) and/or the 2A (RecA-like) domain (residues R179-T260 and L391-V441).

The helicase of the invention preferably comprises a variant of SEQ ID NO: 8 which comprises one or more of (i) I181C; (ii) Y279C; (iii) I281C; and (iv) E288C. The helicase may comprise any combination of (i) to (iv), such as (i); (ii); (iii); (iv); (i) and (ii); (i) and (iii); (i) and (iv); (ii) and (iii); (ii) and (iv); (iii) and (iv); or (i), (ii), (iii) and (iv). The helicase more preferably comprises a variant of SEQ ID NO: 8 which comprises (a) E94C, I281C and A360C or (b) E94C, I281C, G357C and A360C. The helicase of the invention preferably comprises a variant of any one of SEQ ID NOs: 9 to 23 which comprises a cysteine residue at one or more of the position(s) which correspond to those in SEQ ID NO: 8 as defined in (i) to (iv), (a) and (b). The helicase may comprise any of these variants in which Faz is introduced at one or more of the specific positions (or each specific position) instead of cysteine.

The helicase of the invention is further modified to reduce its surface negative charge. Surface residues can be identified in the same way as the Dda domains disclosed above. Surface negative charges are typically surface negatively-charged amino acids, such as aspartic acid (D) and glutamic acid (E).

The helicase is preferably modified to neutralise one or more surface negative charges by substituting one or more negatively charged amino acids with one or more positively charged amino acids, uncharged amino acids, non-polar amino acids and/or aromatic amino acids or by introducing one or more positively charged amino acids, preferably adjacent to one or more negatively charged amino acids. Suitable positively charged amino acids include, but are not limited to, histidine (H), lysine (K) and arginine (R). Uncharged amino acids have no net charge. Suitable uncharged amino acids include, but are not limited to, cysteine (C), serine (S), threonine (T), methionine (M), asparagine (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y).

Preferred substitutions include, but are not limited to, substitution of E with R, substitution of E with K, substitution of E with N, substitution of D with K and substitution of D with R.

The helicase of the invention preferably comprises a variant of SEQ ID NO: 8 and the one or more negatively charged amino acids are one or more of D5, E8, E23, E47, D167, E172, D202, D212 and E273. Any number of these amino acids may be neutralised, such as 1, 2, 3, 4, 5, 6, 7 or 8 of them. Any combination may be neutralised. The helicase of the invention preferably comprises a variant of any one of SEQ ID NOs: 9 to 23 and the one or more negatively charged amino acids correspond to one or more of D5, E8, E23, E47, D167, E172, D202, D212 and E273 in SEQ ID NO: 8. Amino acids in SEQ ID NOs: 9 to 23 which correspond to D5, E8, E23, E47, D167, E172, D202, D212 and E273 in SEQ ID NO: 8 can be determined using the alignment below. The helicase of the invention preferably comprises a variant of SEQ ID NO: 8 which comprises (a) E94C, E273G and A360C or (b) E94C, E273G, N292G and A360C.

The helicase of the invention is preferably further modified by the removal of one or more native cysteine residues. Any number of native cysteine residues may be removed. The number of cysteine residues in each of SEQ ID NOs: 9 to 23 is shown in Table 1 (as #C). The one or more cysteine residues are preferably removed by substitution. The one or more cysteine residues are preferably substituted with alanine (A), serine (S) or valine (V). The helicase of the invention preferably comprises a variant of SEQ ID NO: 8 and the one or more native cysteine residues are one or more of C109, C114, C136, C171 and C412. Any number and combination of these cysteine residues may be removed. For instance, the variant of SEQ ID NO: 8 may comprise C109; C114; C136; C171; C412; C109 and C114; C109 and C136; C109 and C171; C109 and C412; C114 and C136; C114 and C171; C114 and C412; C136 and C171; C136 and C412; C171 and C412; C109, C114 and C136; C109, C114 and C171; C109, C114 and C412; C109, C136 and C171; C109, C136 and C412; C109, C171 and C412; C114, C136 and C171; C114, C136 and C412; C114, C171 and C412; C136, C171 and C412; C109, C114, C136 and C171; C109, C114, C136 and C412; C109, C114, C171 and C412; C109, C136, C171 and C412; C114, C136, C171 and C412; or C109, C114, C136, C171 and C412.

The helicase of the invention is preferably one in which at least one cysteine residue (i.e. one or more cysteine residues) and/or at least one non-natural amino acid (i.e. one or more non-natural amino acids) have been introduced into the tower domain only. Suitable modifications are discussed above.

The helicase of the invention preferably comprises a variant of SEQ ID NO: 8 comprising the following mutations:
E93C and K364C;
E94C and K364C;
E94C and A360C;
L97C and E361C;
L97C and E361C and C412A;
K123C and E361C;
K123C, E361C and C412A;
N155C and K358C;
N155C, K358C and C412A;
N155C and L354C;
N155C, L354C and C412A;
deltaE93, E94C, deltaN95 and A360C;
E94C, deltaN95 and A360C;
E94C, Q100C, I127C and A360C;
L354C;
G357C;
E94C, G357C and A360C;
E94C, Y279C and A360C;
E94C, I281C and A360C;
E94C, Y279Faz and A360C;
Y279C and G357C;
I281C and G357C;
E94C, Y279C, G357C and A360C;
E94C, 1281C, G357C and A360C;
E8R, E47K, E94C, D202K and A360C;
D5K, E23N, E94C, D167K, E172R, D212R and A360C;
D5K, E8R, E23N, E47K, E94C, D167K, E172R, D202K, D212R and A360C;
E94C, C114A, C171A, A360C and C412D;
E94C, C114A, C171A, A360C and C412S;
E94C, C109A, C136A and A360C;
E94C, C109A, C114A, C136A, C171A, A360C and C412S;
E94C, C109V, C114V, C171A, A360C and C412S;
C109A, C114A, C136A, G153C, C171A, E361C and C412A;
C109A, C114A, C136A, G153C, C171A, E361C and C412D;
C109A, C114A, C136A, G153C, C171A, E361C and C412S;
C109A, C114A, C136A, G153C, C171A, K358C and C412A;
C109A, C114A, C136A, G153C, C171A, K358C and C412D
C109A, C114A, C136A, G153C, C171A, K358C and C412S;
C109A, C114A, C136A, N155C, C171A, K358C and C412A;
C109A, C114A, C136A, N155C, C171A, K358C and C412D;
C109A, C114A, C136A, N155C, C171A, K358C and C412S;
C109A, C114A, C136A, N155C, C171A, L354C and C412A;
C109A, C114A, C136A, N155C, C171A, L354C and C412D;
C109A, C114A, C136A, N155C, C171A, L354C and C412S;
C109A, C114A, K123C, C136A, C171A, E361C and C412A;
C109A, C114A, K123C, C136A, C171A, E361C and C412D;
C109A, C114A, K123C, C136A, C171A, E361C and C412S;
C109A, C114A, K123C, C136A, C171A, K358C and C412A;
C109A, C114A, K123C, C136A, C171A, K358C and C412D;
C109A, C114A, K123C, C136A, C171A, K358C and C412S;
C109A, C114A, C136A, G153C, C171A, E361C and C412A;
E94C, C109A, C114A, C136A, C171A, A360C and C412D;
E94C, C109A, C114V, C136A, C171A, A360C and C412D;
E94C, C109V, C114A, C136A, C171A, A360C and C412D;
L97C, C109A, C114A, C136A, C171A, E361C and C412A;
L97C, C109A, C114A, C136A, C171A, E361C and C412D; or
L97C, C109A, C114A, C136A, C171A, E361C and C412S.
   Modifications in the Hook Domain and/or 2A Domain

In one embodiment, the helicase of the invention is one in which at least one cysteine residue and/or at least one non-natural amino acid have been introduced into the hook domain and/or the 2A (RecA-like motor) domain, wherein the helicase has the ability to control the movement of a polynucleotide. At least one cysteine residue and/or at least one non-natural amino acid is preferably introduced into the hook domain and the 2A (RecA-like motor) domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are preferably introduced by substitution. Methods for doing this are known in the art. Suitable modifications of the hook domain and/or the 2A (RecA-like motor) domain are discussed above.

The helicase of the invention is preferably a variant of SEQ ID NO: 8 comprising (a) Y279C, I181C, E288C, Y279C and I181C, (b) Y279C and E288C, (c) 1181C and E288C or (d) Y279C, I181C and E288C. The helicase of the invention preferably comprises a variant of any one of SEQ ID NOs: 9 to 23 which comprises a mutation at one or more of the position(s) which correspond to those in SEQ ID NO: 8 as defined in (a) to (d).

### Surface Modification

In one embodiment, the helicase is modified to reduce its surface negative charge, wherein the helicase has the ability to control the movement of a polynucleotide. Suitable modifications are discussed above. Any number of surface negative charges may be neutralised.

The helicase of the invention preferably comprises a variant of SEQ ID NO: 8 comprising the following mutations:
E273G;
E8R, E47K and D202K;
D5K, E23N, D167K, E172R and D212R; or
D5K, E8R, E23N, E47K, D167K, E172R, D202K and D212R.

### Other Modified Helicases

In one embodiment, the helicase of the invention comprises a variant of SEQ ID NO: 8 comprising:
A360K;
Y92L and/or A360Y;
Y92L, Y350N and Y363N;
Y92L and/or Y363N; or
Y92L.

### Other Modifications

In addition to the specific mutations disclosed above, a variant of SEQ ID NO: 8 may comprise one or more of the following mutations:
K38A;
T91F;
T91N;
T91Q;
T91W;
V96E;
V96F;
V96L
V96Q;
V96R;
V96W;
V96Y;
P274G;
V286F;
V286W;
V286Y;
F291G;
N292F;
N292G;
N292P;
N292Y;
G294Y;
G294F;
K364A; and
W378A.

In addition to the specific mutations disclosed above, a variant of SEQ ID NO: 8 may comprise:

K38A, E94C and A360C;
H64K; E94C and A360C;
H64N; E94C and A360C;
H64Q; E94C and A360C;
H64S; E94C and A360C;
H64W, E94C and A360C;
T80K, E94C and A360C;
T80K, S83K, E94C, N242K, N293K and A360C;
T80K, S83K, E94C, N242K,
N293K, A360C and T394K;
T80K, S83K, E94C, N293K and A360C;
T80K, S83K, E94C, A360C and T394K;
T80K, S83K, E94C, A360C and T394N;
T80K, E94C, N242K and A360C;
T80K, E94C, N242K, N293K and A360C;
T80K, E94C, N293K and A360C;
T80N, E94C and A360C;
H82A, E94C and A360C;
H82A, P89A, E94C, A360C;
H82F, E94C and A360C;
H82Q, E94C, A360C;
H82R, E94C and A360C;
H82W, E94C and A360C;
H82W, P89W, E94C, F98W and A360C;
H82Y, E94C and A360C;
S83K, E94C and A360C;
S83K, T80K, E94C, A360C and T394K;
S83N, E94C and A360C;
S83T, E94C and A360C;
N88H, E94C and A360C;
N88Q, E94C and A360C;
P89A, E94C and A360C;
P89A, F98W, E94C and A360C;
P89A, E94C, F98Y and A360C;
P89A, E94C, F98A and A360C;
P89F, E94C and A360C;
P89S, E94C and A360C;
P89T, E94C and A360C;
P89W, E94C, F98W and A360C;
P89Y, E94C and A360C;
T91F, E94C and A360C;
T91N, E94C and A360C;
T91Q, E94C and A360C;
T91W, E94C and A360C;
E94C, V96E and A360C;
E94C, V96F and A360C;
E94C, V96L and A360C;
F98A and E94C, V96Q and A360C;
E94C, V96R and A360C;
E94C, V96W and A360C;
E94C, V96Y and A360C;
E94C, F98A and A360C;
E94C, F98L and A360C;
E94C, F98V and A360C;
E94C, F98Y and A360C;
E94C; F98W and A360C;
E94C, V150A and A360C;
E94C, V150F and A360C;
E94C, V150I and A360C;
E94C, V150K and A360C;
E94C, V150L and A360C;
E94C, V150S and A360C;
E94C, V150T and A360C;
E94C, V150W and A360C;
E94C, V150Y and A360C;
E94C, F240Y and A360C;
E94C, F240W and A360C;
E94C, N242K and A360C;
E94C, N242K, N293K and A360C;
E94C, P274G and A360C;
E94C, L275G and A360C
E94C, F276A and A360C;
E94C, F276I and A360C;
E94C, F276M and A360C;
E94C, F276V and A360C;
E94C, F276W and A360C;
E94C, F276Y and A360C;
E94C, V286F and A360C;
E94C, V286W and A360C;
E94C, V286Y and A360C;
E94C, S287F and A360C;
E94C, S287W and A360C;
E94C, S287Y and A360C;
E94C, F291G and A360C;
E94C, N292F and A360C;
E94C, N292G and A360C;
E94C, N292P and A360C;
E94C, N292Y and A360C;
E94C, N293F and A360C;
E94C, N293K and A360C;
E94C, N293Q and A360C;
E94C, N293Y and A360C;
E94C, G294F and A360C;
E94C, G294Y and A360C;
E94C, A36C and K364A;
E94C, A360C, W378A;
E94C, A360C and T394K;
E94C, A360C and H396Q;
E94C, A360C and H396S;
E94C, A360C and H396W;
E94C, A360C and Y415F;
E94C, A360C and Y415K;
E94C, A360C and Y415M; or
E94C, A360C and Y415W.

The helicase of the invention preferably comprises a variant of SEQ ID NO: 8 which comprises (a) E94C/A360C/W378A or (b) E94C/A360C/C109A/C136A/W378A or (d) E94C/A360C/C109A/C136A/W378A and then (ΔM1)G1G2 (i.e. deletion of M1 and then addition G1 and G2).

Preferred variants of any one of SEQ ID NOs: 8 to 23 have (in addition to the modifications of the invention) the N-terminal methionine (M) replaced with one glycine residue (G). In the examples this is shown as (ΔM1)G1. It may also be termed M1G. Any of the variants discussed above may further comprise M1G.

The most preferred helicases of the invention comprise a variant of SEQ ID NO: 8 which comprises (a) E94C/F98W/A360C/C109A/C136A/K194L, (b) M1G/E94C/F98W/A360C/C109A/C136A/K194L; (c) E94C/F98W/A360C/C109A/C136A/K199L; or (d) M1G/E94C/F98W/A360C/C109A/C136A/K199L.

### Variants

A variant of a helicase is an enzyme that has an amino acid sequence which varies from that of the wild-type helicase and which has polynucleotide binding activity. In particular, a variant of any one of SEQ ID NOs: 8 to 23 is an enzyme that has an amino acid sequence which varies from that of any one of SEQ ID NOs: 8 to 23 and which has polynucleotide binding activity. Polynucleotide binding activity can be determined using methods known in the art. Suitable methods include, but are not limited to, fluorescence anisotropy, tryptophan fluorescence and electrophoretic mobility shift assay (EMSA). For instance, the ability of a variant to bind a single stranded polynucleotide can be determined as described in the Examples.

The variant has helicase activity. This can be measured in various ways. For instance, the ability of the variant to translocate along a polynucleotide can be measured using electrophysiology, a fluorescence assay or ATP hydrolysis.

The variant may include modifications that facilitate handling of the polynucleotide encoding the helicase and/or facilitate its activity at high salt concentrations and/or room temperature.

Over the entire length of the amino acid sequence of any one of SEQ ID NOs: 8 to 23, a variant will preferably be at least 20% homologous to that sequence based on amino acid similarity or identity. More preferably, the variant polypeptide may be at least 30%, at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 8 to 23 over the entire sequence. There may be at least 70%, for example at least 80%, at least 85%, at least 90% or at least 95%, amino acid identity over a stretch of 100 or more, for example 150, 200, 300, 400 or 500 or more, contiguous amino acids (“hard homology”). Homology is determined as described below. The variant may differ from the wild-type sequence in any of the ways discussed below with reference to SEQ ID NOs: 2 and 4. In particular, in addition to the specific modifications discussed above, the variant of any one of SEQ ID NOs: 8 to 23 may comprise one or more substitutions, one or more deletions and/or one or more additions as discussed below.

Preferred variants of any one of SEQ ID NOs: 8 to 23 have a non-natural amino acid, such as Faz, at the amino- (N-) terminus and/or carboxy (C-) terminus. Preferred variants of any one of SEQ ID NOs: 8 to 23 have a cysteine residue at the amino- (N-) terminus and/or carboxy (C-) terminus. Preferred variants of any one of SEQ ID NOs: 8 to 23 have a cysteine residue at the amino- (N-) terminus and a non-natural amino acid, such as Faz, at the carboxy (C-) terminus or vice versa.

Preferred variants of SEQ ID NO: 8 contain one or more of, such as all of, the following modifications E54G, D151E, 1196N and G357A.

### No Connection

In one preferred embodiment, none of the introduced cysteines and/or non-natural amino acids in a modified helicase of the invention are connected to one another.

Connecting Two More of the Introduced Cysteines and/or Non-Natural Amino Acids

In another preferred embodiment, two more of the introduced cysteines and/or non-natural amino acids in a modified helicase of the invention are connected to one another. This typically reduces the ability of the helicase of the invention to unbind from a polynucleotide.

Any number and combination of two more of the introduced cysteines and/or non-natural amino acids may be connected to one another. For instance, 3, 4, 5, 6, 7, 8 or more cysteines and/or non-natural amino acids may be connected to one another. One or more cysteines may be connected to one or more cysteines. One or more cysteines may be connected to one or more non-natural amino acids, such as Faz. One or more non-natural amino acids, such as Faz, may be connected to one or more non-natural amino acids, such as Faz.

The two or more cysteines and/or non-natural amino acids may be connected in any way. The connection can be transient, for example non-covalent. Even transient connection will reduce unbinding of the polynucleotide from the helicase.

The two or more cysteines and/or non-natural amino acids are preferably connected by affinity molecules. Suitable affinity molecules are known in the art. The affinity molecules are preferably (a) complementary polynucleotides (International Application No. PCT/GB10/000132 (published as WO 2010/086602), (b) an antibody or a fragment thereof and the complementary epitope (Biochemistry 6th Ed, W.H. Freeman and co (2007) pp 953-954), (c) peptide zippers (O'Shea et al., Science 254 (5031): 539-544), (d) capable of interacting by β-sheet augmentation (Remaut and Waksman Trends Biochem. Sci. (2006) 31 436-444), (e) capable of hydrogen bonding, pi-stacking or forming a salt bridge, (f) rotaxanes (Xiang Ma and He Tian Chem. Soc. Rev., 2010, 39, 70-80), (g) an aptamer and the complementary protein (James, W. in Encyclopedia of Analytical Chemistry, R. A. Meyers (Ed.) pp. 4848-4871 John Wiley & Sons Ltd, Chichester, 2000) or (h) half-chelators (Hammerstein et al. J Biol Chem. 2011 April 22; 286(16): 14324-14334). For (e), hydrogen bonding occurs between a proton bound to an electronegative atom and another electronegative atom. Pi-stacking requires two aromatic rings that can stack together where the planes of the rings are parallel. Salt bridges are between groups that can delocalize their electrons over several atoms, e. g. between aspartate and arginine.

The two or more parts may be transiently connected by a hexa-his tag or Ni-NTA.

The two or more cysteines and/or non-natural amino acids are preferably permanently connected. In the context of the invention, a connection is permanent if is not broken while the helicase is used or cannot be broken without intervention on the part of the user, such as using reduction to open —S—S— bonds.

The two or more cysteines and/or non-natural amino acids are preferably covalently-attached. The two or more cysteines and/or non-natural amino acids may be covalently attached using any method known in the art.

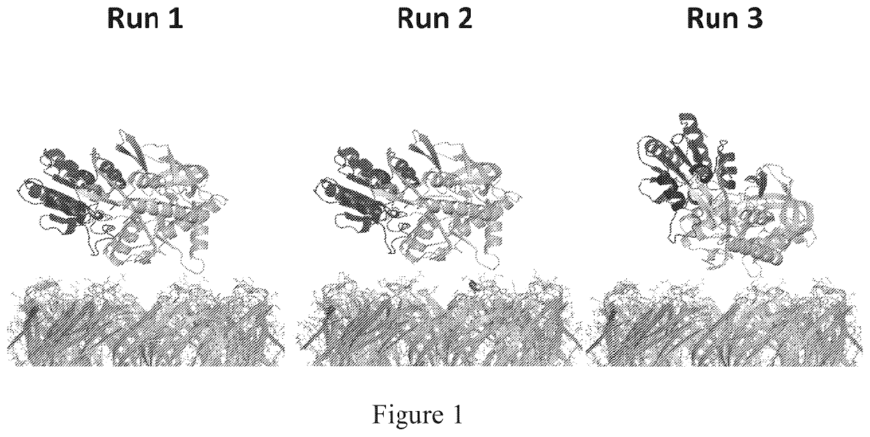

The two or more cysteines and/or non-natural amino acids may be covalently attached via their naturally occurring amino acids, such as cysteines, threonines, serines, aspartates, asparagines, glutamates and glutamines. Naturally occurring amino acids may be modified to facilitate attachment. For instance, the naturally occurring amino acids may be modified by acylation, phosphorylation, glycosylation or farnesylation. Other suitable modifications are known in the art. Modifications to naturally occurring amino acids may be post-translation modifications. The two or more cysteines and/or non-natural amino acids may be attached via amino acids that have been introduced into their sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), any one of the amino acids numbered 1-71 included in FIG. 1 of Liu C. C. and Schultz P. G., Annu. Rev. Biochem., 2010, 79, 413-444 or any one of the amino acids listed below. The introduced amino acids may be modified as discussed above.

In a preferred embodiment, the two or more cysteines and/or non-natural amino acids are connected using linkers. Linker molecules are discussed in more detail below. One suitable method of connection is cysteine linkage. This is discussed in more detail below. The two or more cysteines and/or non-natural amino acids are preferably connected using one or more, such as two or three, linkers. The one or more linkers may be designed to reduce the size of, or close, the opening as discussed above. If one or more linkers are being used to close the opening as discussed above, at least a part of the one or more linkers is preferably oriented such that it is not parallel to the polynucleotide when it is bound by the helicase. More preferably, all of the linkers are oriented in this manner. If one or more linkers are being used to close the opening as discussed above, at least a part of the one or more linkers preferably crosses the opening in an orientation that is not parallel to the polynucleotide when it bound by the helicase. More preferably, all of the linkers cross the opening in this manner. In these embodiments, at least a part of the one or more linkers may be perpendicular to the polynucleotide. Such orientations effectively close the opening such that the polynucleotide cannot unbind from the helicase through the opening.

Each linker may have two or more functional ends, such as two, three or four functional ends. Suitable configurations of ends in linkers are well known in the art.

One or more ends of the one or more linkers are preferably covalently attached to the helicase. If one end is covalently attached, the one or more linkers may transiently connect the two or more cysteines and/or non-natural amino acids as discussed above. If both or all ends are covalently attached, the one or more linkers permanently connect the two or more cysteines and/or non-natural amino acids.

The one or more linkers are preferably amino acid sequences and/or chemical crosslinkers.

Suitable amino acid linkers, such as peptide linkers, are known in the art. The length, flexibility and hydrophilicity of the amino acid or peptide linker are typically designed such that it reduces the size of the opening, but does not to disturb the functions of the helicase. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline. The amino acid sequence of a linker preferably comprises a polynucleotide binding moiety. Such moieties and the advantages associated with their use are discussed below.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to dithiothreitol (DTT).

Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-rnaleimidotriethylene glycol), tris[2-rnaleirnidoethyl]amine (TNMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S—S-PEG3-biotin, DBCO-S—S-PEG3-biotin, DBCO-S—S-PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). The most preferred crosslinker is maleimide-propyl-SRDFWRS-(1,2-diaminoethane)-propyl-maleimide.

The one or more linkers may be cleavable. This is discussed in more detail below.

The two or more cysteines and/or non-natural amino acids may be connected using two different linkers that are specific for each other. One of the linkers is attached to one part and the other is attached to another part. The linkers should react to form a modified helicase of the invention. The two or more cysteines and/or non-natural amino acids may be connected using the hybridization linkers described in International Application No. PCT/GB10/000132 (published as WO 2010/086602). In particular, the two or more cysteines and/or non-natural amino acids may be connected using two or more linkers each comprising a hybridizable region and a group capable of forming a covalent bond. The hybridizable regions in the linkers hybridize and link the two or more cysteines and/or non-natural amino acids. The linked cysteines and/or non-natural amino acids are then coupled via the formation of covalent bonds between the groups. Any of the specific linkers disclosed in International Application No. PCT/GB10/000132 (published as WO 2010/086602) may be used in accordance with the invention.

The two or more cysteines and/or non-natural amino acids may be modified and then attached using a chemical crosslinker that is specific for the two modifications. Any of the crosslinkers discussed above may be used.

The linkers may be labeled. Suitable labels include, but are not limited to, fluorescent molecules (such as Cy3 or AlexaFluor® 555), radioisotopes, e.g. ¹²⁵I, ³⁵S, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting two or more cysteines is via cysteine linkage. This can be mediated by a bi-functional chemical crosslinker or by an amino acid linker with a terminal presented cysteine residue.

The length, reactivity, specificity, rigidity and solubility of any bi-functional linker may be designed to ensure that the size of the opening is reduced sufficiently and the function of the helicase is retained. Suitable linkers include bismaleimide crosslinkers, such as 1,4-bis(maleimido)butane (BMB) or bis(maleimido)hexane. One drawback of bi-functional linkers is the requirement of the helicase to contain no further surface accessible cysteine residues if attachment at specific sites is preferred, as binding of the bi-functional linker to surface accessible cysteine residues may be difficult to control and may affect substrate binding or activity. If the helicase does contain several accessible cysteine residues, modification of the helicase may be required to remove them while ensuring the modifications do not affect the folding or activity of the helicase. This is discussed in International Application No. PCT/GB10/000133 (published as WO 2010/086603). The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S⁻ group. The reactivity of cysteine residues may be protected by thiol protective groups such as 5,5′-dithiobis-(2-nitrobenzoic acid) (dTNB). These may be reacted with one or more cysteine residues of the helicase before a linker is attached. Selective deprotection of surface accessible cysteines may be possible using reducing reagents immobilized on beads (for example immobilized tris(2-carboxyethyl) phosphine, TCEP). Cysteine linkage is discussed in more detail below.

Another preferred method of attachment via Faz linkage. This can be mediated by a bi-functional chemical linker or by a polypeptide linker with a terminal presented Faz residue.

### Other Modifications

The helicase of the invention may also be modified to increase the attraction between (i) the tower domain and (ii) the pin domain and/or the 1A domain. Any known chemical modifications can be made in accordance with the invention. These types of modification are disclosed in PCT/GB2014/052736 (WO 2015/055981).

In particular, the invention provides a helicase of the invention in which at least one charged amino acid has been introduced into (i) the tower domain and/or (ii) the pin domain and/or (iii) the 1A (RecA-like motor) domain, wherein the helicase has the ability to control the movement of a polynucleotide. The ability of the helicase to control the movement of a polynucleotide may be measured as discussed above. The invention preferably provides a helicase of the invention in which at least one charged amino acid has been introduced into (i) the tower domain and (ii) the pin domain and/or the 1A domain.

The at least one charged amino acid may be negatively charged or positively charged. The at least one charged amino acid is preferably oppositely charged to any amino acid(s) with which it interacts in the helicase. For instance, at least one positively charged amino acid may be introduced into the tower domain at a position which interacts with a negatively charged amino acid in the pin domain. The at least one charged amino acid is typically introduced at a position which is not charged in the wild-type (i.e. unmodified) helicase. The at least one charged amino acid may be used to replace at least one oppositely charged amino acid in the helicase. For instance, a positively charged amino acid may be used to replace a negatively charged amino acid.

Suitable charged amino acids are discussed above. The at least one charged amino acid may be natural, such as arginine (R), histidine (H), lysine (K), aspartic acid (D) or glutamic acid (D). Alternatively, the at least one charged amino acid may be artificial or non-natural. Any number of charged amino acids may be introduced into each domain. For instance, 1, 2, 3, 4, 5, 6, 7, 8, 9, 10 or more charged amino acids may be introduced into each domain.

The helicase preferably comprises a variant of SEQ ID NO: 8 which comprises a positively charged amino acid at one or more of the following positions: (i) 93; (ii) 354; (iii) 360; (iv) 361; (v) 94; (vi) 97; (vii) 155; (viii) 357; (ix) 100; and (x) 127. The helicase preferably comprises a variant of SEQ ID NO: 8 which comprises a negatively charged amino acid at one or more of the following positions: (i) 354; (ii) 358; (iii) 360; (iv) 364; (v) 97; (vi) 123; (vii) 155; (viii); 357; (ix) 100; and (x) 127. The helicase preferably comprises a variant of any one of SEQ ID NOs: 9 to 23 which comprises a positively charged amino acid or negatively charged amino acid at the positions which correspond to those in SEQ ID NO: 8 as defined in any of (i) to (x). Positions in any one of SEQ ID NOs: 9 to 23 which correspond to those in SEQ ID NO: 8 can be identified using the alignment of SEQ ID NOs: 8 to 23 below.

The helicase preferably comprises a variant of SEQ ID NO: 8 which is modified by the introduction of at least one charged amino acid such that it comprises oppositely charged amino acid at the following positions: (i) 93 and 354; (ii) 93 and 358; (iii) 93 and 360; (iv) 93 and 361; (v) 93 and 364; (vi) 94 and 354; (vii) 94 and 358; (viii) 94 and 360; (ix) 94 and 361; (x) 94 and 364; (xi) 97 and 354; (xii) 97 and 358; (xiii) 97 and 360; (xiv) 97 and 361; (xv) 97 and 364; (xvi) 123 and 354; (xvii) 123 and 358; (xviii) 123 and 360; (xix) 123 and 361; (xx) 123 and 364; (xxi) 155 and 354; (xxii) 155 and 358; (xxiii) 155 and 360; (xxiv) 155 and 361; (xxv) 155 and 364. The helicase of the invention preferably comprises a variant of any one of SEQ ID NOs: 9 to 23 which comprises oppositely charged amino acids at the positions which correspond to those in SEQ ID NO: 8 as defined in any of (i) to (xxv).

The invention also provides a helicase in which (i) at least one charged amino acid has been introduced into the tower domain and (ii) at least one oppositely charged amino acid has been introduced into the pin domain and/or the 1A (RecA-like motor) domain, wherein the helicase has the ability to control the movement of a polynucleotide. The at least one charged amino acid may be negatively charged and the at least one oppositely charged amino acid may be positively charged or vice versa. Suitable charged amino acids are discussed above. Any number of charged amino acids and any number of oppositely charged amino acids may be introduced. For instance, 1, 2, 3, 4, 5, 6, 7, 8, 9, 10 or more charged amino acids may be introduced and/or 1, 2, 3, 4, 5, 6, 7, 8, 9, 10 or more oppositely charged amino acids may be introduced.

The charged amino acids are typically introduced at positions which are not charged in the wild-type helicase. One or both of the charged amino acids may be used to replace charged amino acids in the helicase. For instance, a positively charged amino acid may be used to replace a negatively charged amino acid. The charged amino acids may be introduced at any of the positions in the (i) tower domain and (ii) pin domain and/or 1A domain discussed above. The oppositely charged amino acids are typically introduced such that they will interact in the resulting helicase. The helicase preferably comprises a variant of SEQ ID NO: 8 in which oppositely charged amino acids have been introduced at the following positions: (i) 97 and 354; (ii) 97 and 360; (iii) 155 and 354; or (iv) 155 and 360. The helicase of the invention preferably comprises a variant of any one of SEQ ID NOs: 9 to 23 which comprises oppositely charged amino acids at the positions which correspond to those in SEQ ID NO: 8 as defined in any of (i) to (iv).

### Construct

The invention also provides a construct comprising a modified helicase of the invention and an additional polynucleotide binding moiety, wherein the helicase is attached to the polynucleotide binding moiety and the construct has the ability to control the movement of a polynucleotide. The construct is artificial or non-natural.

A construct of the invention is a useful tool for controlling the movement of a polynucleotide during Strand Sequencing. A construct of the invention is even less likely than a modified helicase of the invention to disengage from the polynucleotide being sequenced. The construct can provide even greater read lengths of the polynucleotide as it controls the translocation of the polynucleotide through a nanopore.

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

The construct has the ability to control the movement of a polynucleotide. This can be determined as discussed above.

A construct of the invention may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

The helicase may be any of the helicases of the invention discussed above.

The helicase is preferably covalently attached to the additional polynucleotide binding moiety. The helicase may be attached to the moiety at more than one, such as two or three, points.

The helicase can be covalently attached to the moiety using any method known in the art. Suitable methods are discussed above with reference to connecting the two or more parts.

The helicase and moiety may be produced separately and then attached together. The two components may be attached in any configuration. For instance, they may be attached via their terminal (i.e. amino or carboxy terminal) amino acids. Suitable configurations include, but are not limited to, the amino terminus of the moiety being attached to the carboxy terminus of the helicase and vice versa. Alternatively, the two components may be attached via amino acids within their sequences. For instance, the moiety may be attached to one or more amino acids in a loop region of the helicase. In a preferred embodiment, terminal amino acids of the moiety are attached to one or more amino acids in the loop region of a helicase.

In a preferred embodiment, the helicase is chemically attached to the moiety, for instance via one or more linker molecules as discussed above. In another preferred embodiment, the helicase is genetically fused to the moiety. A helicase is genetically fused to a moiety if the whole construct is expressed from a single polynucleotide sequence. The coding sequences of the helicase and moiety may be combined in any way to form a single polynucleotide sequence encoding the construct. Genetic fusion of a pore to a nucleic acid binding protein is discussed in International Application No. PCT/GB09/001679 (published as WO 2010/004265).

The helicase and moiety may be genetically fused in any configuration. The helicase and moiety may be fused via their terminal amino acids. For instance, the amino terminus of the moiety may be fused to the carboxy terminus of the helicase and vice versa. The amino acid sequence of the moiety is preferably added in frame into the amino acid sequence of the helicase. In other words, the moiety is preferably inserted within the sequence of the helicase. In such embodiments, the helicase and moiety are typically attached at two points, i.e. via the amino and carboxy terminal amino acids of the moiety. If the moiety is inserted within the sequence of the helicase, it is preferred that the amino and carboxy terminal amino acids of the moiety are in close proximity and are each attached to adjacent amino acids in the sequence of the helicase or variant thereof. In a preferred embodiment, the moiety is inserted into a loop region of the helicase.

The helicase may be attached directly to the moiety. The helicase is preferably attached to the moiety using one or more, such as two or three, linkers as discussed above. The one or more linkers may be designed to constrain the mobility of the moiety. The helicase and/or the moiety may be modified to facilitate attachment of the one or more linker as discussed above.

Cleavable linkers can be used as an aid to separation of constructs from non-attached components and can be used to further control the synthesis reaction. For example, a hetero-bifunctional linker may react with the helicase, but not the moiety. If the free end of the linker can be used to bind the helicase protein to a surface, the unreacted helicases from the first reaction can be removed from the mixture. Subsequently, the linker can be cleaved to expose a group that reacts with the moiety. In addition, by following this sequence of linkage reactions, conditions may be optimised first for the reaction to the helicase, then for the reaction to the moiety after cleavage of the linker. The second reaction would also be much more directed towards the correct site of reaction with the moiety because the linker would be confined to the region to which it is already attached.

The helicase may be covalently attached to the bifunctional crosslinker before the helicase/crosslinker complex is covalently attached to the moiety. Alternatively, the moiety may be covalently attached to the bifunctional crosslinker before the bifunctional crosslinker/moiety complex is attached to the helicase. The helicase and moiety may be covalently attached to the chemical crosslinker at the same time.

Preferred methods of attaching the helicase to the moiety are cysteine linkage and Faz linkage as described above. In a preferred embodiment, a reactive cysteine is presented on a peptide linker that is genetically attached to the moiety. This means that additional modifications will not necessarily be needed to remove other accessible cysteine residues from the moiety.

Cross-linkage of helicases or moieties to themselves may be prevented by keeping the concentration of linker in a vast excess of the helicase and/or moiety. Alternatively, a “lock and key” arrangement may be used in which two linkers are used. Only one end of each linker may react together to form a longer linker and the other ends of the linker each react with a different part of the construct (i.e. helicase or moiety). This is discussed in more detail below.

The site of attachment is selected such that, when the construct is contacted with a polynucleotide, both the helicase and the moiety can bind to the polynucleotide and control its movement.

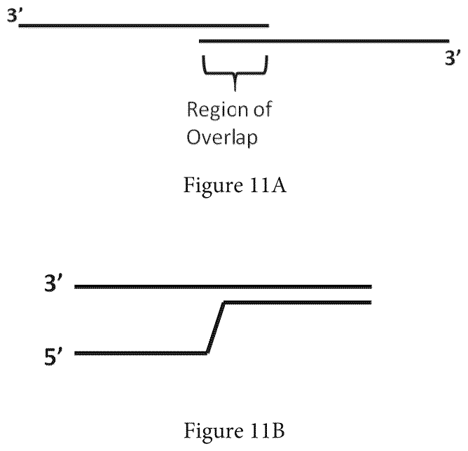

Attachment can be facilitated using the polynucleotide binding activities of the helicase and the moiety. For instance, complementary polynucleotides can be used to bring the helicase and moiety together as they hybridize. The helicase can be bound to one polynucleotide and the moiety can be bound to the complementary polynucleotide. The two polynucleotides can then be allowed to hybridise to each other. This will bring the helicase into close contact with the moiety, making the linking reaction more efficient. This is especially helpful for attaching two or more helicases in the correct orientation for controlling movement of a target polynucleotide. An example of complementary polynucleotides that may be used is shown in FIG. 11A. For helicase-Phi29 constructs the DNA in FIG. 11B could be used.

Tags can be added to the construct to make purification of the construct easier. These tags can then be chemically or enzymatically cleaved off, if their removal is necessary. Fluorophores or chromophores can also be included, and these could also be cleavable.

A simple way to purify the construct is to include a different purification tag on each protein (i.e. the helicase and the moiety), such as a hexa-His-tag and a Strep-Tag®. If the two proteins are different from one another, this method is particularly useful. The use of two tags enables only the species with both tags to be purified easily.

If the two proteins do not have two different tags, other methods may be used. For instance, proteins with free surface cysteines or proteins with linkers attached that have not reacted to form a construct could be removed, for instance using an iodoacetamide resin for maleimide linkers.

Constructs of the invention can also be purified from unreacted proteins on the basis of a different DNA processivity property. In particular, a construct of the invention can be purified from unreacted proteins on the basis of an increased affinity for a polynucleotide, a reduced likelihood of disengaging from a polynucleotide once bound and/or an increased read length of a polynucleotide as it controls the translocation of the polynucleotide through a nanopore

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

### Polynucleotide Binding Moiety

The constructs of the invention comprise a polynucleotide binding moiety. A polynucleotide binding moiety is a polypeptide that is capable of binding to a polynucleotide. The moiety is preferably capable of specific binding to a defined polynucleotide sequence. In other words, the moiety preferably binds to a specific polynucleotide sequence, but displays at least 10 fold less binding to different sequences or more preferably at least 100 fold less binding to different sequences or most preferably at least 1000 fold less binding to different sequences. The different sequence may be a random sequence. In some embodiments, the moiety binds to a specific polynucleotide sequence, but binding to different sequences cannot be measured. Moieties that bind to specific sequences can be used to design constructs that are targeted to such sequences.

The moiety typically interacts with and modifies at least one property of a polynucleotide. The moiety may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated. One or more nucleotides in the target polynucleotide may be damaged. For instance, the polynucleotide may comprise a pyrimidine dimer. Such dimers are typically associated with damage by ultraviolet light and are the primary cause of skin melanomas. One or more nucleotides in the target polynucleotide may be modified, for instance with a label or a tag. Suitable labels are described above. The target polynucleotide may comprise one or more spacers.

A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP) and deoxycytidine monophosphate (dCMP). The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

It is preferred that the tertiary structure of the moiety is known. Knowledge of the three dimensional structure of the moiety allows modifications to be made to the moiety to facilitate its function in the construct of the invention.

The moiety may be any size and have any structure. For instance, the moiety may be an oligomer, such as a dimer or trimer. The moiety is preferably a small, globular polypeptide formed from one monomer. Such moieties are easy to handle and are less likely to interfere with the ability of the helicase to control the movement of the polynucleotide, particularly if fused to or inserted into the sequence of the helicase.

The amino and carboxy terminii of the moiety are preferably in close proximity. The amino and carboxy terminii of the moiety are more preferably presented on same face of the moiety. Such embodiments facilitate insertion of the moiety into the sequence of the helicase. For instance, if the amino and carboxy terminii of the moiety are in close proximity, each can be attached by genetic fusion to adjacent amino acids in the sequence of the helicase.

It is also preferred that the location and function of the active site of the moiety is known. This prevents modifications being made to the active site that abolish the activity of the moiety. It also allows the moiety to be attached to the helicase so that the moiety binds to the polynucleotide and controls its movement. Knowledge of the way in which a moiety may bind to and orient polynucleotides also allows an effective construct to be designed.

The constructs of the invention are useful in Strand Sequencing. The moiety preferably binds the polynucleotide in a buffer background which is compatible with Strand Sequencing and the discrimination of the nucleotides. The moiety preferably has at least residual activity in a salt concentration well above the normal physiological level, such as from 100 mM to 2M. The moiety is more preferably modified to increase its activity at high salt concentrations. The moiety may also be modified to improve its processivity, stability and shelf life.

Suitable modifications can be determined from the characterisation of polynucleotide binding moieties from extremphiles such as halophilic, moderately halophilic bacteria, thermophilic and moderately thermophilic organisms, as well as directed evolution approaches to altering the salt tolerance, stability and temperature dependence of mesophilic or thermophilic exonucleases.

The polynucleotide binding moiety preferably comprises one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

The helix-hairpin-helix (HhH) domains are polypeptide motifs that bind DNA in a sequence non-specific manner. They have been shown to confer salt stability and processivity when fused to polymerases, as well as increasing their thermal stability. Suitable domains include domain H (residues 696-751) and domain HI (residues 696-802) from Topoisomerase V from Methanopyrus kandleri (SEQ ID NO: 47). As discussed below, the polynucleotide binding moiety may be domains H-L of SEQ ID NO: 47 as shown in SEQ ID NO: 48. Topoisomerase V from Methanopyrus kandleri is an example of a double-stranded binding protein as discussed below.

The HhH domain preferably comprises the sequence shown in SEQ ID NO: 24 or 37 or 38 or a variant thereof. This domain increases the processivity and the salt tolerance of a helicase when used in a construct of the invention. A variant of SEQ ID NO: 24 or 37 or 38 is a protein that has an amino acid sequence which varies from that of SEQ ID NO: 24 or 37 or 38 and which has polynucleotide binding activity. This can be measured as described above. A variant typically has at least 50% homology to SEQ ID NO: 24 or 37 or 38 based on amino acid identity over its entire sequence (or any of the % homologies discussed above in relation to helicases) and has polynucleotide binding activity. A variant may differ from SEQ ID NO: 24 or 37 or 38 in any of the ways discussed above in relation to helicases or below in relation to pores. A variant preferably comprises one or more substituted cysteine residues and/or one or more substituted Faz residues to facilitate attachment to the helicase as discussed above.

SSBs bind single stranded DNA with high affinity in a sequence non-specific manner. They exist in all domains of life in a variety of forms and bind DNA either as monomers or multimers. Using amino acid sequence alignment and logarithms (such as Hidden Markov models) SSBs can be classified according to their sequence homology. The Pfam family, PF00436, includes proteins that all show sequence similarity to known SSBs. This group of SSBs can then be further classified according to the Structural Classification of Proteins (SCOP). SSBs fall into the following lineage: Class; All beta proteins, Fold; OB-fold, Superfamily: Nucleic acid-binding proteins, Family; Single strand DNA-binding domain, SSB. Within this family SSBs can be classified according to subfamilies, with several type species often characterised within each subfamily.

The SSB may be from a eukaryote, such as from humans, mice, rats, fungi, protozoa or plants, from a prokaryote, such as bacteria and archaea, or from a virus.

Eukariotic SSBs are known as replication protein A (RPAs). In most cases, they are hetero-trimers formed of different size units. Some of the larger units (e.g. RPA70 of Saccharomyces cerevisiae) are stable and bind ssDNA in monomeric form.

Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). The SSBs from archaeal genomes are considered to be related with eukaryotic RPAs. Few of them, such as the SSB encoded by the crenarchaeote Sulfolobus solfataricus, are homo-tetramers. The SSBs from most other species are closer related to the replication proteins from eukaryotes and are referred to as RPAs. In some of these species they have been shown to be monomeric (Methanococcus jannaschii and Methanothermobacter thermoautotrophicum). Still, other species of Archaea, including Archaeoglobus fulgidus and Methanococcoides burtonii, appear to each contain two open reading frames with sequence similarity to RPAs. There is no evidence at protein level and no published data regarding their DNA binding capabilities or oligomeric state. However, the presence of two oligonucleotide/oligosaccharide (OB) folds in each of these genes (three OB folds in the case of one of the M. burtonii ORFs) suggests that they also bind single stranded DNA.

Viral SSBs bind DNA as monomers. This, as well as their relatively small size renders them amenable to genetic fusion to other proteins, for instance via a flexible peptide linker. Alternatively, the SSBs can be expressed separately and attached to other proteins by chemical methods (e.g. cysteines, unnatural amino-acids). This is discussed in more detail below.

The SSB is preferably either (i) an SSB comprising a carboxy-terminal (C-terminal) region which does not have a net negative charge or (ii) a modified SSB comprising one or more modifications in its C-terminal region which decreases the net negative charge of the C-terminal region. Such SSBs do not block the transmembrane pore and therefore allow characterization of the target polynucleotide.

Examples of SSBs comprising a C-terminal region which does not have a net negative charge include, but are not limited to, the human mitochondrial SSB (HsmtSSB; SEQ ID NO: 39, the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 40) or a variant of any of those proteins. A variant is a protein that has an amino acid sequence which varies from that of the wild-type protein and which has single stranded polynucleotide binding activity. Polynucleotide binding activity can be determined using methods known in the art (and as described above). For instance, the ability of a variant to bind a single stranded polynucleotide can be determined as described in the Examples.

A variant of SEQ ID NO: 39 or 40 typically has at least 50% homology to SEQ ID NO: 39 or 40 based on amino acid identity over its entire sequence (or any of the % homologies discussed above in relation to helicases) and has single stranded polynucleotide binding activity. A variant may differ from SEQ ID NO: 39 or 40 in any of the ways discussed above in relation to helicases. In particular, a variant may have one or more conservative substitutions as shown in Tables 7 and 8.

Examples of SSBs which require one or more modifications in their C-terminal region to decrease the net negative charge include, but are not limited to, the SSB of E. coli (EcoSSB; SEQ ID NO: 41, the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDC13 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 42), the SSB from RB69 (gp32; SEQ ID NO: 25), the SSB from T7 (gp2.5; SEQ ID NO: 26) or a variant of any of these proteins. Hence, the SSB used in the method of the invention may be derived from any of these proteins.

In addition to the one or more modifications in the C-terminal region, the SSB used in the method may include additional modifications which are outside the C-terminal region or do not decrease the net negative charge of the C-terminal region. In other words, the SSB used in the method of the invention is derived from a variant of a wild-type protein. A variant is a protein that has an amino acid sequence which varies from that of the wild-type protein and which has single stranded polynucleotide binding activity. Polynucleotide binding activity can be determined as discussed above.

The SSB used in the invention may be derived from a variant of SEQ ID NO: 25, 26, 41 or 42. In other words, a variant of SEQ ID NO: 25, 26, 41 or 42 may be used as the starting point for the SSB used in the invention, but the SSB actually used further includes one or more modifications in its C-terminal region which decreases the net negative charge of the C-terminal region. A variant of SEQ ID NO: 25, 26, 41 or 42 typically has at least 50% homology to SEQ ID NO: 25, 26, 41 or 42 based on amino acid identity over its entire sequence (or any of the % homologies discussed above in relation to helicases) and has single stranded polynucleotide binding activity. A variant may differ from SEQ ID NO: 25, 26, 41 or 42 in any of the ways discussed above in relation to helicases. In particular, a variant may have one or more conservative substitutions as shown in Tables 7 and 8.

It is straightforward to identify the C-terminal region of the SSB in accordance with normal protein N to C nomenclature. The C-terminal region of the SSB is preferably about the last third of the SSB at the C-terminal end, such as the last third of the SSB at the C-terminal end. The C-terminal region of the SSB is more preferably about the last quarter, fifth or eighth of the SSB at the C-terminal end, such as the last quarter, fifth or eighth of the SSB at the C-terminal end. The last third, quarter, fifth or eighth of the SSB may be measured in terms of numbers of amino acids or in terms of actual length of the primary structure of the SSB protein. The length of the various amino acids in the N to C direction are known in the art.

The C-terminal region is preferably from about the last 10 to about the last 60 amino acids of the C-terminal end of the SSB. The C-terminal region is more preferably about the last 15, about the last 20, about the last 25, about the last 30, about the last 35, about the last 40, about the last 45, about the last 50 or about the last 55 amino acids of the C-terminal end of the SSB.

The C-terminal region typically comprises a glycine and/or proline rich region. This proline/glycine rich region gives the C-terminal region flexibility and can be used to identify the C-terminal region.

Suitable modifications for decreasing the net negative charge are disclosed in International Application No. PCT/GB2013/051924 (published as WO 2014/013259). The SSB may be any of the SSBs disclosed in this International application.

The modified SSB most preferably comprises a sequence selected from those shown in SEQ ID NOs: 33, 34, 43 to 46.

Double-stranded binding proteins bind double stranded DNA with high affinity. Suitable double-stranded binding proteins include, but are not limited to Mutator S (MutS; NCBI Reference Sequence: NP_417213.1; SEQ ID NO: 49), Sso7d (Sulfolobus solfataricus P2; NCBI Reference Sequence: NP_343889.1; SEQ ID NO: 50; Nucleic Acids Research, 2004, Vol 32, No. 3, 1197-1207), Sso10b1 (NCBI Reference Sequence: NP_342446.1; SEQ ID NO: 51), Sso10b2 (NCBI Reference Sequence: NP_342448.1; SEQ ID NO: 52), Tryptophan repressor (Trp repressor; NCBI Reference Sequence: NP_291006.1; SEQ ID NO: 53), Lambda repressor (NCBI Reference Sequence: NP_040628.1; SEQ ID NO: 54), Cren7 (NCBI Reference Sequence: NP_342459.1; SEQ ID NO: 55), major histone classes H1/H5, H2A, H2B, H3 and H4 (NCBI Reference Sequence: NP_066403.2, SEQ ID NO: 56), dsbA (NCBI Reference Sequence: NP_049858.1; SEQ ID NO: 57), Rad51 (NCBI Reference Sequence: NP_002866.2; SEQ ID NO: 58), sliding clamps and Topoisomerase V Mka (SEQ ID NO: 47) or a variant of any of these proteins. A variant of SEQ ID NO: 47, 49, 50, 51, 52, 53, 54, 55, 56, 57 or 58 typically has at least 50% homology to SEQ ID NO: 47, 49, 50, 51, 52, 53, 54, 55, 56, 57 or 58 based on amino acid identity over its entire sequence (or any of the % homologies discussed above in relation to helicases) and has single stranded polynucleotide binding activity. A variant may differ from SEQ ID NO: 47, 49, 50, 51, 52, 53, 54, 55, 56, 57 or 58 in any of the ways discussed above in relation to helicases. In particular, a variant may have one or more conservative substitutions as shown in Tables 7 and 8. Most polymerases achieve processivity by interacting with sliding clamps. In general, these are multimeric proteins (homo-dimers or homo-trimers) that encircle dsDNA. These sliding clamps require accessory proteins (clamp loaders) to assemble them around the DNA helix in an ATP-dependent process. They also do not contact DNA directly, acting as a topological tether. As sliding clamps interact with their cognate polymerases in a specific manner via a polymerase domain, this fragment could be fused to the helicase in order to incite recruitment of helicases onto the sliding clamp. This interaction could be further stabilized by the generation of a covalent bond (introduction of cysteines or unnatural amino-acids).

Related to DNA sliding clamps, processivity factors are viral proteins that anchor their cognate polymerases to DNA, leading to a dramatic increase in the length of the fragments generated. They can be monomeric (as is the case for UL42 from Herpes simplex virus 1) or multimeric (UL44 from Cytomegalovirus is a dimer), they do not form closed rings around the DNA strand and they contact DNA directly. UL42 has been shown to increase processivity without reducing the rate of its corresponding polymerase, suggesting that it interacts with DNA in a different mode to SSBs. The UL42 preferably comprises the sequence shown in SEQ ID NO: 27 or SEQ ID NO: 32 or a variant thereof. A variant of SEQ ID NO: 27 or 32 is a protein that has an amino acid sequence which varies from that of SEQ ID NO: 27 or 32 and which has polynucleotide binding activity. This can be measured as described above. A variant typically has at least 50% homology to SEQ ID NO: 27 or 32 based on amino acid identity over its entire sequence (or any of the % homologies discussed above in relation to helicases) and has polynucleotide binding activity. A variant may differ from SEQ ID NO: 27 or SEQ ID NO: 32 in any of the ways discussed above in relation to helicases or below in relation to pores. A variant preferably comprises one or more substituted cysteine residues and/or one or more substituted Faz residues to facilitate attachment to the helicase as discussed above.

Attaching UL42 to a helicase could be done via genetic fusion or chemical attachment (cysteines, unnatural amino-acids). As the polymerase polypeptide that binds UL42 is visible in the crystal structure, these 35 amino acids (residues 1200-1235) could be fused onto the C-terminus of the helicase and the natural affinity between this polypeptide and the processivity factor used to form a complex. The interaction could be stabilized by introducing a covalent interaction (cysteines or unnatural amino-acids). One option is to utilize a natural UL42 cysteine (C300) that is located close to the polypeptide interaction site and introduce a point mutation into the polymerase polypeptide (e.g. L1234C).

A reported method of increasing polymerase processivity is by exploiting the interaction between E. coli thioredoxin (Trx) and the thioredoxin binding domain (TBD) of bacteriophage T7 DNA polymerase (residues 258-333). The binding of Trx to TBD causes the polypeptide to change conformation to one that binds DNA. TBD is believed to clamp down onto a DNA strand and limit the polymerase off-rate, thus increasing processivity. Chimeric polymerases have been made by transferring TBD onto a non-processive polymerase, resulting in 1000 fold increase in polymerised fragment length. There were no attempts to attach TBD to any other class of proteins, but a covalent link between TBD and Trx was engineered and can be used to stabilise the interaction.

Some helicases use accessory proteins in-vivo to achieve processivity (e.g. cisA from phage Φx174 and geneII protein from phage M13 for E. coli Rep helicase). Some of these proteins have been shown to interact with more than one helicase (e.g. MutL acts on both UvrD and Rep, though not to the same extent). These proteins have intrinsic DNA binding capabilities, some of them recognizing a specific DNA sequence. The ability of some of these accessory proteins to covalently attach themselves to a specific DNA sequence could also be used to create a set starting point for the helicase activity.

The proteins that protect the ends of chromosomes bind to telomeric ssDNA sequences in a highly specific manner. This ability could either be exploited as is or by using point mutations to abolish the sequence specificity.

Small DNA binding motifs (such as helix-turn-helix) recognize specific DNA sequences. In the case of the bacteriophage 434 repressor, a 62 residue fragment was engineered and shown to retain DNA binding abilities and specificity.

An abundant motif in eukaryotic proteins, zinc fingers consist of around 30 amino-acids that bind DNA in a specific manner. Typically each zinc finger recognizes only three DNA bases, but multiple fingers can be linked to obtain recognition of a longer sequence.

Proliferating cell nuclear antigens (PCNAs) form a very tight clamp (doughnut) which slides up and down the dsDNA or ssDNA. The PCNA from crenarchaeota is unique in being a hetero-trimer so it is possible to functionalise one subunit and retain activity. Its subunits are shown in SEQ ID NOs: 28, 29 and 30. The PCNA is preferably a trimer comprising the sequences shown in SEQ ID NOs: 28, 29 and 30 or variants thereof. PCNA sliding clamp (NCBI Reference Sequence: ZP_06863050.1; SEQ ID NO: 59) forms a dimer. The PCNA is preferably a dimer comprising SEQ ID NO: 59 or a variant thereof. A variant is a protein that has an amino acid sequence which varies from that of SEQ ID NO: 28, 29, 30 or 59 and which has polynucleotide binding activity. This can be measured as described above. A variant is typically a trimer comprising sequences that have at least 50% homology to SEQ ID NOs: 28, 29 and 30 or a dimer comprising sequences that have at least 50% homology to SEQ ID NO: 59 based on amino acid identity over each entire sequence (or any of the % homologies discussed above in relation to helicases) and which has polynucleotide binding activity. A variant may comprise sequences which differ from SEQ ID NO: 28, 29, 30 or 59 in any of the ways discussed above in relation to helicases or below in relation to pores. A variant preferably comprises one or more substituted cysteine residues and/or one or more substituted Faz residues to facilitate attachment to the helicase as discussed above. In a preferred embodiment, subunits 1 and 2 of the PCNA from crenarchaeota (i.e. SEQ ID NOs: 28 and 29 or variants thereof) are attached, such as genetically fused, and the resulting protein is attached to a helicase to form a construct of the invention. During use of the construct, subunit 3 (i.e. SEQ ID NO: 30 or a variant thereof) may be added to complete the PCNA clamp (or doughnut) once the construct has bound the polynucleotide. In a preferred embodiment, one monomer of the PCNA sliding clamp (i.e. SEQ ID NO: 59 or a variant thereof) is attached, such as genetically fused, to a helicase to form a construct of the invention. During use of the construct, the second monomer (i.e. SEQ ID NO: 59 or a variant thereof) may be added to complete the PCNA clamp (or doughnut) once the construct has bound the polynucleotide.

The polynucleotide binding motif may be selected from any of those shown in Table 5 below.

TABLE 5
Suitable polynucleotide binding motifs

| | | | | | | | MW | |
|---|---|---|---|---|---|---|---|---|
| No. | Name | Class | Organism | Structure | Sequence | Functional form | (Da) | Notes |
| 1 | SSBEco | ssb | Escherichia coli | 1QVC, | P0AGE0 | homo-tetramer | 18975 | |
| | | | | 1EYG | | | | |
| 2 | SSBBhe | ssb | Bartonella | 3LGJ, | Q6G302 | homo-tetramer | 16737 | structure only |
| | | | henselae | 3PGZ | | | | |
| 3 | SSBCbu | ssb | Coxiella burnetii | 3TQY | Q83EP4 | homo-tetramer | 17437 | structure only |
| 4 | SSBTma | ssb | Thermathoga | 1Z9F | Q9WZ73 | homo-dimer | 16298 | small, |
| | | | maritima | | | | | thermostable, |
| | | | | | | | | salt independent |
| | | | | | | | | DNA binding |
| 5 | SSBHpy | ssb | Helicobacter | 2VW9 | O25841 | homo-tetramer | 20143 | |
| | | | pylori | | | | | |
| 6 | SSBDra | ssb | Deinococcus | 1SE8 | Q9RY51 | homo-dimer | 32722 | |
| | | | radiodurans | | | | | |
| 7 | SSBTaq | ssb | Thermus | 2FXQ | Q9KH06 | homo-dimer | 30026 | |
| | | | aquaticus | | | | | |
| 8 | SSBMsm | ssb | Mycobacterium | 3A5U, 1X3E | Q9AFI5 | homo-tetramer | 17401 | tetramer more |
| | | | smegmatis | | | | | stable than |
| | | | | | | | | E. coli, binding |
| | | | | | | | | less salt |
| | | | | | | | | dependent |
| 9 | SSBSso | ssb/RPA | Sulfolobus | 1O7I | Q97W73 | homo-tetramer | 16138 | similarities with |
| | | | solfataricus | | | | | RPA |
| 10 | SSBMHsmt | ssb | Homo sapiens | 3ULL | Q04837 | homo-tetramer | 17260 | |
| 11 | SSBMle | ssb | Mycobacterium | 3AFP | P46390 | homo-tetramer | 17701 | |
| | | | leprae | | | | | |
| 12 | gp32T4 | ssb | Bacteriohage T4 | 1GPC | P03695 | monomer | 33506 | Homo-dimer in |
| | | | | | | | | the absence of |
| | | | | | | | | DNA, monomer |
| | | | | | | | | when binding |
| | | | | | | | | DNA. |
| 13 | gp32RB69 | ssb | Bacteriophage | 2A1K | Q7Y265 | monomer | 33118 | |
| | | | RB69 | | | | | |
| 14 | gp2.5T7 | ssb | Bacteriohage T7 | 1JE5 | P03696 | monomer | 25694 | |
| 15 | UL42 | processivity | Herpes virus 1 | 1DML | P10226 | monomer | 51159 | binds ssDNA |
| | | factor | | | | | | dsDNA, |
| | | | | | | | | structure shows |
| | | | | | | | | link with |
| | | | | | | | | polymerase |
| 16 | UL44 | processivity | Herpes virus 5 | 1YYP | P16790 | homo-dimer | 46233 | forms C shaped |
| | | factor | (cytomegalovirus) | | | | | clamp on DNA |
| 17 | pf8 | processivity | KSHV | 3I2M | Q77ZG5 | homo-dimer | 42378 | |
| | | factor | | | | | | |
| 18 | RPAMja | RPA | Methanococcus | 3DM3 | Q58559 | monomer | 73842 | contains 4 OB |
| | | | jannaschii | | | | | folds. Structure |
| | | | | | | | | of fragment |
| 19 | RPAMma | RPA | Methanococcus | 3E0E, | Q6LYF9 | monomer | 71388 | Core domain |
| | | | maripaludis | 2K5V | | | | structure |
| 20 | RPAMth | RPA | Methanothermobacter | | | monomer | 120000 | Shown to |
| | | | thermoautotrophicus | | | | | interact directly |
| | | | | | | | | with Hel308. |
| | | | | | | | | Sequence from |
| | | | | | | | | paper. |
| 21 | RPA70Sce | RPA | Saccharomyces | 1YNX | P22336 | hetero-trimer | 70348 | unit has two OB |
| | | | cerevisiae | | | | | folds and binds |
| | | | | | | | | DNA |
| 22 | RPAMbu1 | RPA | Methanococcoides | | Q12V72 | ? | 41227 | three OB folds |
| | | | burtonii | | | | | identified |
| 23 | RPAMbu2 | RPA | Methanococcoides | | Q12W96 | ? | 47082 | two OB folds |
| | | | burtonii | | | | | identified |
| 24 | RPA70Hsa | RPA | Homo sapiens | 1JMC | P27694 | hetero-trimer | 68138 | |
| 25 | RPA14Hsa | RPA | Homo sapiens | 3KDF | P35244 | hetero-trimer | 13569 | in complex with |
| | | | | | | | | RPA32 |
| 26 | gp45T4 | sliding | Bacteriophage | 1CZD | P04525 | homo-trimer | 24858 | ring shape |
| | | clamp | T4 | | | | | threads DNA |
| 27 | BetaEco | sliding | E. coli | 3BEP | P0A988 | homo-dimer | 40587 | ring shape |
| | | clamp | | | | | | threads DNA, |
| | | | | | | | | may bind ssDNA |
| | | | | | | | | in pocket |
| 28 | PCNASce | sliding | Saccharomyces | 1PLQ, 3K4X | P15873 | homo-dimer | 28916 | ring shape |
| | | clamp | cerevisiae | | | | | threads DNA |
| 29 | PCNATko | sliding | Thermococcus | 3LX1 | Q5JF32 | homo-dimer | 28239 | |
| | | clamp | kodakaraensis | | | | | |
| 30 | PCNAHvo | sliding | Haloferax | 3IFV | D0VWY8 | homo-dimer | 26672 | |
| | | clamp | volcanii | | | | | |
| 31 | PCNAPfu | sliding | Pyrococcus | 1GE8 | O73947 | homo-dimer | 28005 | |
| | | clamp | furiosus | | | | | |
| 32 | PCNAMbu | sliding | Methanococcoides | | Q12U18 | homo-dimer | 27121 | Inferred from |
| | | clamp | burtonii | | | | | homology |
| 33 | BetaMtu | sliding | Mycobacterium | 3P16 | Q50790 | homo-dimer | 42113 | |
| | | clamp | tuberculosis | | | | | |
| 34 | Beta Tma | sliding | Thermotoga | 1VPK | Q9WYA0 | homo-dimer | 40948 | |
| | | clamp | maritima | | | | | |
| 35 | BetaSpy | sliding | Streptococcus | 2AVT | Q9EVR1 | homo-dimer | 41867 | |
| | | clamp | pyrogenes | | | | | |
| 36 | gp45RB69 | sliding | Bacteriophage | 1B77 | O80164 | homo-trimer | 25111 | Structure shows |
| | | clamp | RB69 | | | | | interaction with |
| | | | | | | | | polypeptide from |
| | | | | | | | | polymerase |
| 37 | p55Hsa | DNA | Homo sapiens | 2G4C, | Q9UHN | monomer | 54911 | interacts with |
| | | binding | (mitochondrial) | 3IKL, | | | | specific |
| | | protein | | 3IKM | | | | polymerase |
| | | | | | | | | domain |
| 38 | p55Dme | DNA | Drosophylla | | Q9VJV8 | monomer | 41027 | associates with |
| | | binding | melanogaster | | | | | polymerase |
| | | protein | | | | | | Gamma |
| | | | | | | | | conferring salt |
| | | | | | | | | tolerance, |
| | | | | | | | | processivity and |
| | | | | | | | | increased activity |
| 39 | p55Xla | DNA | Xenopus laevis | | Q9W6G7 | monomer | 52283 | |
| | | binding | | | | | | |
| | | protein | | | | | | |
| 40 | RepDSau | replication | Staphylococcus | | P08115 | homo-dimer | 37874 | increases |
| | | initiation | aureus | | | | | processivity of |
| | | protein | | | | | | PcrA, covalently |
| | | | | | | | | and specifically |
| | | | | | | | | links DNA |
| 41 | G2P | replication | Enterobacteria | | P69546 | monomer | 46168 | increases |
| | | initiation | phage 1 | | | | | processivity of |
| | | protein | | | | | | Rep, covalently |
| | | | | | | | | and specifically |
| | | | | | | | | links DNA |
| 42 | MUtLEco | mismatch | Escherichia coli | 1BKN, | P23367 | homo-dimer | 67924 | increases |
| | | repair | | 1B62, | | | | processivity of |
| | | protein | | 1B63 | | | | UvrD (and Rep) |
| 43 | KuMtu | DNA | Mycobacterium | | O05866 | homo-dimer | 30904 | increases |
| | | repair | tuberculosis | | | | | processivity of |
| | | protein | | | | | | UvrD1. Structure |
| | | | | | | | | available for |
| | | | | | | | | human Ku |
| 44 | OnTEBP | telomere | Oxytricha nova- | 1OTC | P29549 | hetero-dimer | 56082 | Specific biding |
| | | binding | Alpha | | | | | to 3′ end |
| | | protein | | | | | | T4G4T4G4. |
| | | | | | | | | Alpha subunit |
| | | | | | | | | may be enough |
| | | | Oxytricha nova- | | P16458 | | 41446 | |
| | | | Beta | | | | | |
| 45 | EcrTEBP | telomere | Euplotes crassus | | Q06183 | monomer | 53360 | Homolog to |
| | | binding | | | | | | OnTEBP with no |
| | | protein | | | | | | Beta subunit in |
| | | | | | | | | genome |
| 46 | TteTEBP | telomere | Tetrachymena | | Q23FB9 | hetero-dimer | 53073 | Homolog to |
| | | binding | termophila Alpha | | | | | OnTEBP-Alpha |
| | | protein | | | | | | |
| | | | Tetrachymena | | Q23FH0 | | 54757 | May be homolog |
| | | | termophila Beta | | | | | to OnTEBP Beta |
| 47 | pot1Spo | telomere | Schizosaccharomyces | | O13988 | monomer | 64111 | related to TEBP |
| | | binding | pombe | | | | | |
| | | proteins | | | | | | |
| 48 | Cdc13pSce | telomere | Saccharomyces | | C7GSV7 | monomer | 104936 | specific binding |
| | | binding | cerevisiae | | | | | to telomeric |
| | | proteins | | | | | | DNA |
| 49 | C1 | repressor | Bacteriophage | | P16117 | homo-dimer | 10426 | binds DNA |
| | | | 434 | | | | | specifically as |
| | | | | | | | | homo-dimer |
| 50 | LexA | repressor | Escherichia coli | 1LEB | P0A7C2 | homo-dimer | 22358 | binds DNA |
| | | | | | | | | specifically as |
| | | | | | | | | homo-dimer |

The polynucleotide binding moiety is preferably derived from a polynucleotide binding enzyme. A polynucleotide binding enzyme is a polypeptide that is capable of binding to a polynucleotide and interacting with and modifying at least one property of the polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide binding moiety does not need to display enzymatic activity as long as it is capable of binding the polynucleotide and controlling its movement. For instance, the moiety may be derived from an enzyme that has been modified to remove its enzymatic activity or may be used under conditions which prevent it from acting as an enzyme.

The polynucleotide binding moiety is preferably derived from a nucleolytic enzyme. The enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are exonucleases, polymerases, helicases and topoisomerases, such as gyrases. Suitable exonucleases include, but are not limited to, exonuclease I from E. coli, exonuclease III enzyme from E. coli, RecJ from T. thermophilus and bacteriophage lambda exonuclease, TatD exonuclease and variants thereof.

The polymerase is preferably a member of any of the Moiety Classification (EC) groups 2.7.7.6, 2.7.7.7, 2.7.7.19, 2.7.7.48 and 2.7.7.49. The polymerase is preferably a DNA-dependent DNA polymerase, an RNA-dependent DNA polymerase, a DNA-dependent RNA polymerase or an RNA-dependent RNA polymerase. The polymerase may be PyroPhage® 3173 DNA Polymerase (which is commercially available from Lucigen® Corporation), SD Polymerase (commercially available from Bioron®) or variants thereof. The polynucleotide binding moiety is preferably derived from Phi29 DNA polymerase (SEQ ID NO: 31). The moiety may comprise the sequence shown in SEQ ID NO: 101 or a variant thereof. A variant of SEQ ID NO: 31 is an enzyme that has an amino acid sequence which varies from that of SEQ ID NO: 31 and which has polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NO: 31, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 31 over the entire sequence. There may be at least 80%, for example at least 85%, 90% or 95%, amino acid identity over a stretch of 200 or more, for example 230, 250, 270 or 280 or more, contiguous amino acids (“hard homology”). Homology is determined as described below. The variant may differ from the wild-type sequence in any of the ways discussed below with reference to SEQ ID NOs: 2 and 4.

The helicase may be any of those discussed above, including any of SEQ ID NOs: 8 to 23. Helicase dimers and multimers are discussed in detail below. The polynucleotide binding moiety may be a polynucleotide binding domain derived from a helicase. For instance, the polynucleotide binding moiety preferably comprises the sequence shown in SEQ ID NOs: 35 or 36 or a variant thereof. A variant of SEQ ID NOs: 35 or 36 is a protein that has an amino acid sequence which varies from that of SEQ ID NOs: 35 or 36 and which has polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NOs: 35 or 36, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOs: 35 or 36 over the entire sequence. There may be at least 80%, for example at least 85%, 90% or 95%, amino acid identity over a stretch of 40 or more, for example 50, 60, 70 or 80 or more, contiguous amino acids (“hard homology”). Homology is determined as described below. The variant may differ from the wild-type sequence in any of the ways discussed below with reference to SEQ ID NOs: 2 and 4.

The topoisomerase is preferably a member of any of the Moiety Classification (EC) groups 5.99.1.2 and 5.99.1.3.

The polynucleotide binding moiety may be any of the enzymes discussed above.

The moiety may be labelled with a revealing label. The label may be any of those described above.

The moiety may be isolated from any moiety-producing organism, such as E. coli, T. thermophilus or bacteriophage, or made synthetically or by recombinant means. For example, the moiety may be synthesized by in vitro translation and transcription as described below. The moiety may be produced in large scale following purification as described below.

### Helicase Oligomers

As will be clear from the discussion above, the polynucleotide binding moiety is preferably derived from a helicase. For instance, it may be a polynucleotide domain from a helicase. The moiety more preferably comprises one or more helicases. The helicases may be any of those discussed above with reference to the constructs of the invention, including the helicases of the invention and helicases which are not modified in accordance with the invention. In such embodiments, the constructs of the invention of course comprise two or more helicases attached together. At least one of the helicases is modified in accordance with the invention. The constructs may comprise two, three, four, five or more helicases. In other words, the constructs of the invention may comprise a helicase dimer, a helicase trimer, a helicase tetramer, a helicase pentamer and the like.

The two or more helicases can be attached together in any orientation. Identical or similar helicases may be attached via the same amino acid position or spatially proximate amino acid positions in each helicase. This is termed the “head-to-head” formation. Alternatively, identical or similar helicases may be attached via positions on opposite or different sides of each helicase. This is termed the “head-to-tail” formation. Helicase trimers comprising three identical or similar helicases may comprise both the head-to-head and head-to-tail formations.

The two or more helicases may be different from one another (i.e. the construct is a hetero-dimer, -trimer, -tetramer or -pentamer etc.). For instance, the constructs of the invention may comprise (a) one or more helicases of the invention and one or more helicases which are not modified in accordance with the invention or (b) two or more different helicases of the invention. The construct may comprise two different variants of the same Dda helicase. For instance, the construct may comprise two variants of one of the helicases discussed above with one or more cysteine residues or Faz residues introduced at different positions in each variant. In this instance, the helicases can be in a head-to-tail formation.

Hetero-dimers can be formed in two possible ways. The first involves the use of a homo-bifunctional linker as discussed above. One of the helicase variants can be modified with a large excess of linker in such a way that one linker is attached to one molecule of the protein. This linker modified variant can then be purified away from unmodified proteins, possible homo-dimers and unreacted linkers to react with the other helicase variant. The resulting dimer can then be purified away from other species.

The second involves the use of hetero-bifunctional linkers. For example, one of the helicase variants can be modified with a first PEG linker containing maleimide or iodoacetamide functional group at one end and a cyclooctyne functional group (DIBO) at the other end. An example of this is shown below:

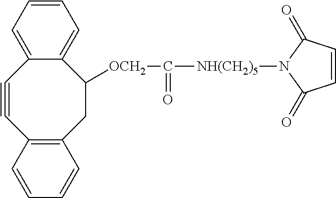

The second helicase variant can be modified with a second PEG linker containing maleimide or iodoacetamide functional group at one end and an azide functional group at the other end. An example is shown below:

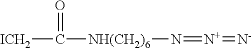

The two helicase variants with two different linkers can then be purified and clicked together (using copper free click chemistry) to make a dimer. Copper free click chemistry has been used in these applications because of its desirable properties. For example, it is fast, clean and not poisonous towards proteins. However, other suitable bio-orthogonal chemistries include, but are not limited to, Staudinger chemistry, hydrazine or hydrazide/aldehyde or ketone reagents (HyNic+4FB chemistry, including all Solulink™ reagents), Diels-Alder reagent pairs and boronic acid/salicyhydroxamate reagents.

These two ways of linking two different variants of the same helicase are also valid for any of the constructs discussed above in which the helicase and the moiety are different from one another, such as dimers of two different helicases and a helicase-polymerase dimer.

Similar methodology may also be used for linking different Faz variants. One Faz variant can be modified with a large excess of linker in such a way that one linker is attached to one molecule of the protein. This linker modified Faz variant can then be purified away from unmodified proteins, possible homo-dimers and unreacted linkers to react with the second Faz variant. The resulting dimer can then be purified away from other species.

Hetero-dimers can also be made by linking cysteine variants and Faz variants of the same helicase or different helicases. Hetero-bifunctional PEG linkers with maleimide or iodoacetamide functionalities at one end and DBCO functionality at the other end can be used in this combination of mutants. An example of such a linker is shown below (DBCO-PEG4-maleimide):

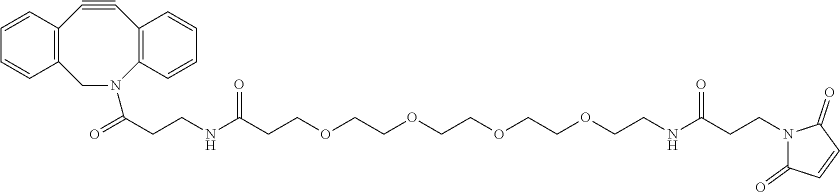 [SMILES: O=C(CCOCCOCCOCCOCCNC(=O)CCN1C(=O)C=CC1=O)NCCC(=O)N1Cc2ccccc2C#Cc2ccccc21]

The length of the linker can be varied by changing the number of PEG units between the two functional groups.

Helicase hetero-trimers can comprise three different types of helicases. The same is true for oligomers comprising more than three helicases. The two or more helicases within a construct may be different variants of the same helicase, such as different variants of any one of SEQ ID NOs: 8 to 23. The different variants may be modified at different positions to facilitate attachment via the different positions. The hetero-trimers may therefore be in a head-to-tail and head-to-head formation.

The two or more helicases in the constructs of the invention may be the same as one another (i.e. the construct is a homo-dimer, -trimer, -tetramer or -pentamer etc.) In such embodiments, the helicases are preferably attached using the same position in each helicase. The helicases are therefore attached head-to-head. The helicases may be linked using a cysteine residue or a Faz residue that has been substituted into the helicases at the same position. Cysteine residues in identical helicase variants can be linked using a homo-bifunctional linker containing thiol reactive groups such as maleimide or iodoacetamide. These functional groups can be at the end of a polyethyleneglycol (PEG) chain as in the following example:

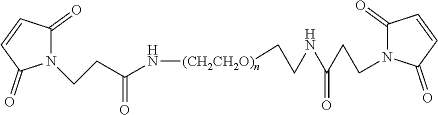

The length of the linker can be varied to suit the required applications. For example, n can be 2, 3, 4, 8, 11, 12, 16 or more. PEG linkers are suitable because they have favourable properties such as water solubility. Other non PEG linkers can also be used in cysteine linkage.

By using similar approaches, identical Faz variants can also be made into homo-dimers. Homo-bifunctional linkers with DIBO functional groups can be used to link two molecules of the same Faz variant to make homo-dimers using Cu²⁺, free click chemistry. An example of a linker is given below:

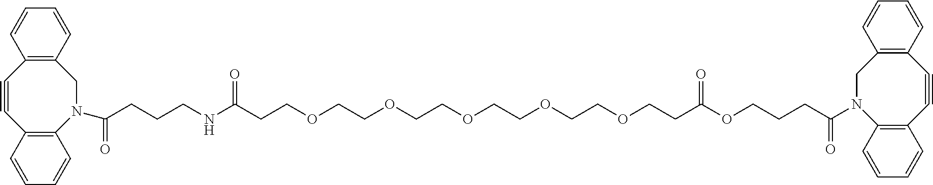 [SMILES: O=C(CCOCCOCCOCCOCCOCCC(=O)OCCCC(=O)N1Cc2ccccc2C#Cc2ccccc21)NCCCC(=O)N1Cc2ccccc2C#Cc2ccccc21]

The length of the PEG linker can vary to include 2, 4, 8, 12, 16 or more PEG units. Such linkers can also be made to incorporate a florescent tag to ease quantifications. Such fluorescence tags can also be incorporated into Maleimide linkers.

Homo-dimers or longer homo-oligomers may also be prepared in the head-to-tail formation if two or more cysteine residues or non-natural amino acids are introduced in the helicase in accordance with the invention and different cysteines or non-natural amino acids in the different helicase monomers are attached together. For instance, homo-oligomers may be formed from variants of SEQ ID NO: 8 comprising Y279C and G357C and the C at 279 in one monomer may be attached to the C at 357 in another monomer. Similarly, homo-oligomers may be formed from variants of SEQ ID NO: 8 comprising 1281C and G357C and the C at 281 in one monomer may be attached to the C at 357 in another monomer. The same is true when Faz is introduced at these positions instead of C. Such C and Faz mutants allow series or trains of helicases to be created.

### Polynucleotide Sequences

The invention provides a polynucleotide comprising a sequence which encodes a helicase of the invention or a construct of the invention. The polynucleotide may consist of such a sequence. The polynucleotide may be any of those discussed above.

Any of the proteins described herein may be expressed using methods known in the art. Polynucleotide sequences may be isolated and replicated using standard methods in the art. Chromosomal DNA may be extracted from a helicase producing organism, such as Methanococcoides burtonii, and/or a SSB producing organism, such as E. coli. The gene encoding the sequence of interest may be amplified using PCR involving specific primers. The amplified sequences may then be incorporated into a recombinant replicable vector such as a cloning vector. The vector may be used to replicate the polynucleotide in a compatible host cell. Thus polynucleotide sequences may be made by introducing a polynucleotide encoding the sequence of interest into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into a suitable expression vector. In an expression vector, the polynucleotide sequence is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence will be chosen to be compatible with the expression vector used to transform the cell. The host cell is typically bacterial and preferably E. coli. Any cell with a λ DE3 lysogen, for example Rosetta2(DE3)pLys, C41 (DE3), BL21 (DE3), JM109 (DE3), B834 (DE3), TUNER, Origami and Origami B, can express a vector comprising the T7 promoter.

### Series

The invention also provides a series of two or more helicases attached (or bound) to a polynucleotide, wherein at least one of the two or more helicases is a helicase of the invention. The series may comprise any number of helicases such as 2, 3, 4, 5, 6, 7, 8, 9, 10 or more helicases. Any number of the helicases may be helicases of the invention. All of the two or more helicases are preferably helicases of the invention. The one or more helicases of the invention may be any of those discussed above.

The two or more helicases may be the same helicase or may be different helicases. For instance, if the series comprises two or more helicases of the invention, they may be the same or may be different.

The series may comprise any number and any combination of helicases of the invention. The series of two or more helicases preferably comprises at least two helicases of the invention. The series may comprise two or more helicases each of which comprises a variant of SEQ ID NO: 8 comprising (or only comprising) (a) P89F, (b) V150I, (c) V150H, (d) P89F and F98W, (e) P89F and V150I, (f) P89F and V150H, (g) F98W and V150I, (h) F98W and V150H (i) P89F, F98W and V150I or (j) P89F, F98W and V150H.

The series may comprise two or more helicases each of which comprises a variant of SEQ ID NO: 8 comprising (i) E94C/A360C, (ii) E94C/A360C and then (ΔM1)G1G2 (i.e. deletion of M1 and then addition G1 and G2), (iii) E94C/A360C/C109A/C136A, (iv) E94C/A360C/C109A/C136A and then (ΔM1)G1G2 (i.e. deletion of M1 and then addition G1 and G2), (v) E94C/A360C/W378A, (vi) E94C/A360C/W378A and then (ΔM1)G1G2 (i.e. deletion of M1 and then addition G1 and G2), (vii) E94C/A360C/C109A/C136A/W378A or (viii) E94C/A360C/C109A/C136A/W378A and then (ΔM1)G1G2 (i.e. deletion of M1 and then addition G1 and G2). One helicase of the invention in the series preferably comprises a variant of SEQ ID NO: 8 comprising one of (i) to (iv) and another helicase of the invention in the series preferably comprises a variant of SEQ ID NO: 8 comprising one of (v) to (viii).

In addition to one or more helicases of the invention, the series may comprise one or more helicases which are not part of the invention. The one or more helicases may be or be derived from a Hel308 helicase, a RecD helicase, such as TraI helicase or a TrwC helicase, a XPD helicase or a Dda helicase (such as any one of SEQ ID NOs: 8 to 23). The one or more helicases may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013/098561); PCT/GB2013/051925 (published as WO 2014/013260); PCT/GB2013/051924 (published as WO 2014/013259) and PCT/GB2013/051928 (published as WO 2014/013262); and PCT/GB2014/052736 (WO 2015/055981). In particular, the one or more helicases are preferably modified to reduce the size of an opening in the polynucleotide binding domain through which in at least one conformational state the polynucleotide can unbind from the helicase. This is disclosed in WO 2014/013260.

The two or more helicases in the series may be separate from one another. The two or more helicases in the series may be brought together by a transmembrane pore as the polynucleotide moves through the pore. The two or more helicases in the series may contact one another.

The two or more helicases are preferably not attached to one another except via the polynucleotide. The two or more helicases are preferably not covalently attached to one another.

The two or more helicases may be attached or covalently attached to one another. The helicases may be attached in any order and using any method. A series of attached helicases may be called a train.

Polynucleotides to which the series of the invention may be attached/bound are discussed in more detail below.

### Methods of the Invention

The invention provides a method of controlling the movement of a target polynucleotide. The method comprises contacting the target polynucleotide with a modified helicase of the invention or a construct of the invention and thereby controlling the movement of the polynucleotide. The method is preferably carried out with a potential applied across the pore. As discussed in more detail below, the applied potential typically results in the formation of a complex between the pore and the helicase or construct. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11; 129(27):8650-5.

The invention also provides a method of characterising a target polynucleotide. The method comprises (a) contacting the target polynucleotide with a transmembrane pore and a modified helicase of the invention or a construct of the invention such that the helicase or construct controls the movement of the target polynucleotide through the pore. The method also comprises (b) taking one or more measurements as the polynucleotide moves with respect to the pore wherein the measurements are indicative of one or more characteristics of the target polynucleotide and thereby characterising the target polynucleotide.

In all of the methods of the invention, the helicases or constructs may be any of those discussed above.

Any number of helicases of the invention may be used in these methods. For instance, 1, 2, 3, 4, 5, 6, 7, 8, 9, 10 or more helicases may be used. If two or more helicases of the invention are used, they may be the same or different. Suitable numbers and combinations are discussed above with reference to the series of the invention. These equally apply to the methods of the invention.

If two or more helicases are used, they may be attached to one another. The two or more helicases may be covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259) and PCT/GB2013/051928 (published as WO 2014/013262); and PCT/GB2014/052736 (WO 2015/055981).

If two or more helicases are used, they are preferably not attached to one another except via the polynucleotide. The two or more helicases are more preferably not covalently attached to one another.

Steps (a) and (b) are preferably carried out with a potential applied across the pore as discussed above. In some instances, the current passing through the pore as the polynucleotide moves with respect to the pore is used to determine the sequence of the target polynucleotide. This is Strand Sequencing.

The method of the invention is for characterising a target polynucleotide. A polynucleotide is defined above.

The whole or only part of the target polynucleotide may be characterised using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The sample may be a non-biological sample. The non-biological sample is preferably a fluid sample. Examples of a non-biological sample include surgical fluids, water such as drinking water, sea water or river water, and reagents for laboratory tests.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well in the membrane along which or into which hydrated ions may flow.

Any transmembrane pore may be used in the invention. The pore may be biological or artificial. Suitable pores include, but are not limited to, protein pores, polynucleotide pores and solid state pores.

Any membrane may be used in accordance with the invention. Suitable membranes are well-known in the art. The membrane is preferably an amphiphilic layer. An amphiphilic layer is a layer formed from amphiphilic molecules, such as phospholipids, which have both at least one hydrophilic portion and at least one lipophilic or hydrophobic portion. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer.

The amphiphilic layer may be a monolayer or a bilayer. The amphiphilic layer is typically a planar lipid bilayer or a supported bilayer.

The amphiphilic layer is typically a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Examples. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial lipid bilayer. The layer may comprise other transmembrane and/or intramembrane proteins as well as other molecules in addition to the pore. Suitable apparatus and conditions are discussed below. The method of the invention is typically carried out in vitro.

The polynucleotide may be coupled to the membrane. The polynucleotide is preferably coupled to the membrane using one or more anchors. The polynucleotide may be coupled to the membrane using any known method.

Each anchor comprises a group which couples (or binds) to the polynucleotide and a group which couples (or binds) to the membrane. Each anchor may covalently couple (or bind) to the polynucleotide and/or the membrane.

Each polynucleotide may be coupled to the membrane using any number of anchors, such as 2, 3, 4 or more anchors. For instance, one polynucleotide may be coupled to the membrane using two anchors each of which separately couples (or binds) to both the polynucleotide and membrane.

The one or more anchors may comprise the one or more helicases and/or the one or more molecular brakes.

If the membrane is an amphiphilic layer, such as a copolymer membrane or a lipid bilayer, the one or more anchors preferably comprise a polypeptide anchor present in the membrane and/or a hydrophobic anchor present in the membrane. The hydrophobic anchor is preferably a lipid, fatty acid, sterol, carbon nanotube, polypeptide, protein or amino acid, for example cholesterol, palmitate or tocopherol. In preferred embodiments, the one or more anchors are not the detector.

The components of the membrane, such as the amphiphilic molecules, copolymer or lipids, may be chemically-modified or functionalised to form the one or more anchors. Examples of suitable chemical modifications and suitable ways of functionalising the components of the membrane are discussed in more detail below. Any proportion of the membrane components may be functionalized, for example at least 0.01%, at least 0.1%, at least 1%, at least 10%, at least 25%, at least 50% or 100%.

The polynucleotide may be coupled directly to the membrane. The one or more anchors used to couple the polynucleotide to the membrane preferably comprise a linker. The one or more anchors may comprise one or more, such as 2, 3, 4 or more, linkers. One linker may be used couple more than one, such as 2, 3, 4 or more, polynucleotides to the membrane.

Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The polynucleotide may hybridise to a complementary sequence on the circular polynucleotide linker.

The one or more anchors or one or more linkers may comprise a component that can be cut to broken down, such as a restriction site or a photolabile group.

Functionalised linkers and the ways in which they can couple molecules are known in the art. For instance, linkers functionalised with maleimide groups will react with and attach to cysteine residues in proteins. In the context of this invention, the protein may be present in the membrane or may be used to couple (or bind) to the polynucleotide. This is discussed in more detail below.

Crosslinkage of polynucleotides can be avoided using a “lock and key” arrangement. Only one end of each linker may react together to form a longer linker and the other ends of the linker each react with the polynucleotide or membrane respectively. Such linkers are described in International Application No. PCT/GB10/000132 (published as WO 2010/086602).

The use of a linker is preferred in the sequencing embodiments discussed below. If a polynucleotide is permanently coupled directly to the membrane in the sense that it does not uncouple when interacting with the pore, then some sequence data will be lost as the sequencing run cannot continue to the end of the polynucleotide due to the distance between the membrane and the detector. If a linker is used, then the polynucleotide can be processed to completion.

The coupling may be permanent or stable. In other words, the coupling may be such that the polynucleotide remains coupled to the membrane when interacting with the pore.

The coupling may be transient. In other words, the coupling may be such that the polynucleotide may decouple from the membrane when interacting with the pore.

For certain applications, such as aptamer detection, the transient nature of the coupling is preferred. If a permanent or stable linker is attached directly to either the 5′ or 3′ end of a polynucleotide and the linker is shorter than the distance between the membrane and the transmembrane pore's channel, then some sequence data will be lost as the sequencing run cannot continue to the end of the polynucleotide. If the coupling is transient, then when the coupled end randomly becomes free of the membrane, then the polynucleotide can be processed to completion. Chemical groups that form permanent/stable or transient links are discussed in more detail below. The polynucleotide may be transiently coupled to an amphiphilic layer or triblock copolymer membrane using cholesterol or a fatty acyl chain. Any fatty acyl chain having a length of from 6 to 30 carbon atom, such as hexadecanoic acid, may be used.

In preferred embodiments, a polynucleotide, such as a nucleic acid, is coupled to an amphiphilic layer such as a triblock copolymer membrane or lipid bilayer. Coupling of nucleic acids to synthetic lipid bilayers has been carried out previously with various different tethering strategies. These are summarised in Table 6 below.

TABLE 6

| Anchor | Type of | |
|---|---|---|
| comprising | coupling | Reference |
| Thiol | Stable | Yoshina-Ishii, C. and S. G. Boxer (2003). |
| | | “Arrays of mobile tethered vesicles on |
| | | supported lipid bilayers.” J Am Chem Soc |
| | | 125(13): 3696-7. |
| Biotin | Stable | Nikolov, V., R. Lipowsky, et al. (2007). |
| | | “Behavior of giant vesicles with anchored |
| | | DNA molecules.” Biophys J 92(12): 4356-68 |
| Cholesterol | Transient | Pfeiffer, I. and F. Hook (2004). “Bivalent |
| | | cholesterol-based coupling of oligonucletides |
| | | to lipid membrane assemblies.” J Am Chem |
| | | Soc 126(33): 10224-5 |
| Surfactant | Stable | van Lengerich, B., R. J. Rawle, et al. |
| (e.g. Lipid, | | “Covalent attachment of lipid vesicles to a |
| Palmitate, | | fluid-supported bilayer allows observation of |
| etc) | | DNA-mediated vesicle interactions.” |
| | | Langmuir 26(11): 8666-72 |

Synthetic polynucleotides and/or linkers may be functionalised using a modified phosphoramidite in the synthesis reaction, which is easily compatible for the direct addition of suitable anchoring groups, such as cholesterol, tocopherol, palmitate, thiol, lipid and biotin groups. These different attachment chemistries give a suite of options for attachment to polynucleotides. Each different modification group couples the polynucleotide in a slightly different way and coupling is not always permanent so giving different dwell times for the polynucleotide to the membrane. The advantages of transient coupling are discussed above.

Coupling of polynucleotides to a linker or to a functionalised membrane can also be achieved by a number of other means provided that a complementary reactive group or an anchoring group can be added to the polynucleotide. The addition of reactive groups to either end of a polynucleotide has been reported previously. A thiol group can be added to the 5′ of ssDNA or dsDNA using T4 polynucleotide kinase and ATPTS (Grant, G. P. and P. Z. Qin (2007). “A facile method for attaching nitroxide spin labels at the 5′ terminus of nucleic acids.” Nucleic Acids Res 35(10): e77). An azide group can be added to the 5′-phosphate of ssDNA or dsDNA using T4 polynucleotide kinase and 7-[2-Azidoethyl]-ATP or 7-[6-Azidohexyl]-ATP. Using thiol or Click chemistry a tether, containing either a thiol, iodoacetamide OPSS or maleimide group (reactive to thiols) or a DIBO (dibenzocyclooxtyne) or alkyne group (reactive to azides), can be covalently attached to the polynucleotide. A more diverse selection of chemical groups, such as biotin, thiols and fluorophores, can be added using terminal transferase to incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169(2): 376-82). Streptavidin/biotin and/or streptavidin/desthiobiotin coupling may be used for any other polynucleotide. The Examples below describes how a polynucleotide can be coupled to a membrane using streptavidin/biotin and streptavidin/desthiobiotin. It may also be possible that anchors may be directly added to polynucleotides using terminal transferase with suitably modified nucleotides (e.g. cholesterol or palmitate).

The one or more anchors preferably couple the polynucleotide to the membrane via hybridisation. Hybridisation in the one or more anchors allows coupling in a transient manner as discussed above. The hybridisation may be present in any part of the one or more anchors, such as between the one or more anchors and the polynucleotide, within the one or more anchors or between the one or more anchors and the membrane. For instance, a linker may comprise two or more polynucleotides, such as 3, 4 or 5 polynucleotides, hybridised together. The one or more anchors may hybridise to the polynucleotide. The one or more anchors may hybridise directly to the polynucleotide or directly to a Y adaptor and/or leader sequence attached to the polynucleotide or directly to a bridging moiety adaptor, such as a hairpin loop adaptor, attached to the polynucleotide (as discussed below). Alternatively, the one or more anchors may be hybridised to one or more, such as 2 or 3, intermediate polynucleotides (or “splints”) which are hybridised to the polynucleotide, to a Y adaptor and/or leader sequence attached to the polynucleotide or to a bridging moiety adaptor attached to the polynucleotide (as discussed below).

The one or more anchors may comprise a single stranded or double stranded polynucleotide. One part of the anchor may be ligated to a single stranded or double stranded polynucleotide. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89(20): 9823-5). Alternatively, either a single stranded or double stranded polynucleotide can be ligated to a double stranded polynucleotide and then the two strands separated by thermal or chemical denaturation. To a double stranded polynucleotide, it is possible to add either a piece of single stranded polynucleotide to one or both of the ends of the duplex, or a double stranded polynucleotide to one or both ends. For addition of single stranded polynucleotides to the a double stranded polynucleotide, this can be achieved using T4 RNA ligase I as for ligation to other regions of single stranded polynucleotides. For addition of double stranded polynucleotides to a double stranded polynucleotide then ligation can be “blunt-ended”, with complementary 3′ dA/dT tails on the polynucleotide and added polynucleotide respectively (as is routinely done for many sample prep applications to prevent concatemer or dimer formation) or using “sticky-ends” generated by restriction digestion of the polynucleotide and ligation of compatible adapters. Then, when the duplex is melted, each single strand will have either a 5′ or 3′ modification if a single stranded polynucleotide was used for ligation or a modification at the 5′ end, the 3′ end or both if a double stranded polynucleotide was used for ligation.

If the polynucleotide is a synthetic strand, the one or more anchors can be incorporated during the chemical synthesis of the polynucleotide. For instance, the polynucleotide can be synthesised using a primer having a reactive group attached to it.

Adenylated polynucleotides are intermediates in ligation reactions, where an adenosine-monophosphate is attached to the 5′-phosphate of the polynucleotide. Various kits are available for generation of this intermediate, such as the 5′ DNA Adenylation Kit from NEB. By substituting ATP in the reaction for a modified nucleotide triphosphate, then addition of reactive groups (such as thiols, amines, biotin, azides, etc) to the 5′ of a polynucleotide can be possible. It may also be possible that anchors could be directly added to polynucleotides using a 5′ DNA adenylation kit with suitably modified nucleotides (e.g. cholesterol or palmitate).

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. Single or multiple nucleotides can be added to 3′ end of single or double stranded DNA by employing a polymerase. Examples of polymerases which could be used include, but are not limited to, Terminal Transferase, Klenow and E. coli Poly(A) polymerase). By substituting ATP in the reaction for a modified nucleotide triphosphate then anchors, such as a cholesterol, thiol, amine, azide, biotin or lipid, can be incorporated into double stranded polynucleotides. Therefore, each copy of the amplified polynucleotide will contain an anchor.

Ideally, the polynucleotide is coupled to the membrane without having to functionalise the polynucleotide. This can be achieved by coupling the one or more anchors, such as a polynucleotide binding protein or a chemical group, to the membrane and allowing the one or more anchors to interact with the polynucleotide or by functionalizing the membrane. The one or more anchors may be coupled to the membrane by any of the methods described herein. In particular, the one or more anchors may comprise one or more linkers, such as maleimide functionalised linkers.

In this embodiment, the polynucleotide is typically RNA, DNA, PNA, TNA or LNA and may be double or single stranded. This embodiment is particularly suited to genomic DNA polynucleotides.

The one or more anchors can comprise any group that couples to, binds to or interacts with single or double stranded polynucleotides, specific nucleotide sequences within the polynucleotide or patterns of modified nucleotides within the polynucleotide, or any other ligand that is present on the polynucleotide.

Suitable binding proteins for use in anchors include, but are not limited to, E. coli single stranded binding protein, P5 single stranded binding protein, T4 gp32 single stranded binding protein, the TOPO V dsDNA binding region, human histone proteins, E. coli HU DNA binding protein and other archaeal, prokaryotic or eukaryotic single stranded or double stranded polynucleotide (or nucleic acid) binding proteins, including those listed below.

The specific nucleotide sequences could be sequences recognised by transcription factors, ribosomes, endonucleases, topoisomerases or replication initiation factors. The patterns of modified nucleotides could be patterns of methylation or damage.

The one or more anchors can comprise any group which couples to, binds to, intercalates with or interacts with a polynucleotide. The group may intercalate or interact with the polynucleotide via electrostatic, hydrogen bonding or Van der Waals interactions. Such groups include a lysine monomer, poly-lysine (which will interact with ssDNA or dsDNA), ethidium bromide (which will intercalate with dsDNA), universal bases or universal nucleotides (which can hybridise with any polynucleotide) and osmium complexes (which can react to methylated bases). A polynucleotide may therefore be coupled to the membrane using one or more universal nucleotides attached to the membrane. Each universal nucleotide may be coupled to the membrane using one or more linkers. The universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl (C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-O′-methylinosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. The universal nucleotide more preferably comprises 2′-deoxyinosine. The universal nucleotide is more preferably IMP or dIMP. The universal nucleotide is most preferably dPMP (2′-Deoxy-P-nucleoside monophosphate) or dKMP (N6-methoxy-2, 6-diaminopurine monophosphate).

The one or more anchors may couple to (or bind to) the polynucleotide via Hoogsteen hydrogen bonds (where two nucleobases are held together by hydrogen bonds) or reversed Hoogsteen hydrogen bonds (where one nucleobase is rotated through 1800 with respect to the other nucleobase). For instance, the one or more anchors may comprise one or more nucleotides, one or more oligonucleotides or one or more polynucleotides which form Hoogsteen hydrogen bonds or reversed Hoogsteen hydrogen bonds with the polynucleotide. These types of hydrogen bonds allow a third polynucleotide strand to wind around a double stranded helix and form a triplex. The one or more anchors may couple to (or bind to) a double stranded polynucleotide by forming a triplex with the double stranded duplex.

In this embodiment at least 1%, at least 10%, at least 25%, at least 50% or 100% of the membrane components may be functionalized.

Where the one or more anchors comprise a protein, they may be able to anchor directly into the membrane without further functonalisation, for example if it already has an external hydrophobic region which is compatible with the membrane. Examples of such proteins include, but are not limited to, transmembrane proteins, intramembrane proteins and membrane proteins. Alternatively the protein may be expressed with a genetically fused hydrophobic region which is compatible with the membrane. Such hydrophobic protein regions are known in the art.

The one or more anchors are preferably mixed with the polynucleotide before contacting with the membrane, but the one or more anchors may be contacted with the membrane and subsequently contacted with the polynucleotide.

In another aspect the polynucleotide may be functionalised, using methods described above, so that it can be recognised by a specific binding group. Specifically the polynucleotide may be functionalised with a ligand such as biotin (for binding to streptavidin), amylose (for binding to maltose binding protein or a fusion protein), Ni-NTA (for binding to poly-histidine or poly-histidine tagged proteins) or a peptides (such as an antigen).

According to a preferred embodiment, the one or more anchors may be used to couple a polynucleotide to the membrane when the polynucleotide is attached to a leader sequence which preferentially threads into the pore. Leader sequences are discussed in more detail below. Preferably, the polynucleotide is attached (such as ligated) to a leader sequence which preferentially threads into the pore. Such a leader sequence may comprise a homopolymeric polynucleotide or an abasic region. The leader sequence is typically designed to hybridise to the one or more anchors either directly or via one or more intermediate polynucleotides (or splints). In such instances, the one or more anchors typically comprise a polynucleotide sequence which is complementary to a sequence in the leader sequence or a sequence in the one or more intermediate polynucleotides (or splints). In such instances, the one or more splints typically comprise a polynucleotide sequence which is complementary to a sequence in the leader sequence.

An example of a molecule used in chemical attachment is EDC (1-ethyl-3-[3-dimethylaminopropyl]carbodiimide hydrochloride). Reactive groups can also be added to the 5′ of polynucleotides using commercially available kits (Thermo Pierce, Part No. 22980). Suitable methods include, but are not limited to, transient affinity attachment using histidine residues and Ni-NTA, as well as more robust covalent attachment by reactive cysteines, lysines or non natural amino acids.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as at least 6, at least 7, at least 8 or at least 9 subunits. The pore is preferably made up of 6, 7, 8 or 9 subunits. The pore is preferably a hexameric, heptameric, octameric or nonameric pore. The pore may be a homo-oligomer or a hetero-oligomer.

The transmembrane protein pore typically comprises a barrel or channel through which the ions may flow. The subunits of the pore typically surround a central axis and contribute strands to a transmembrane R barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from lysenin. Suitable pores derived from lysenin are disclosed in International Application No. PCT/GB2013/050667 (published as WO 2013/153359). Suitable pores derived from CsgG are disclosed in International Application No. PCT/EP2015/069965. The transmembrane pore may be derived from Msp or from α-hemolysin (α-HL).

The transmembrane protein pore is preferably derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp typically comprises the sequence shown in SEQ ID NO: 2 or a variant thereof. SEQ ID NO: 2 is wild-type MspA monomer. A variant of SEQ ID NO: 2 is a polypeptide that has an amino acid sequence which varies from that of SEQ ID NO: 2 and which has the ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid similarity or identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP, BLAST and PSIBLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

The variant may comprise the following mutations: D90N, D91N, D93N, D118R, D134R and E139K. The variant may be any of the variants disclosed in International Application No. PCT/GB2012/050301 (WO 2012/107778).

The variant preferably (a) does not comprise aspartic acid (D) at position 90; (b) does not comprise aspartic acid (D) at position 91; (c) comprises aspartic acid (D) or glutamic acid (E) at position 93; and (d) comprises one or more modifications which decrease the net negative charge of the inward facing amino acids in the cap forming region and/or the barrel forming region of the monomer. Preferred mutations in (d) include, but are not limited to, D118R, Q126R, D134R and E139K. The variant preferably comprises D90N, D91N, D or E at 93, D118R, D134R and E139K. The variant may be any of the variants disclosed in International Application No. PCT/GB2015/051290.

SEQ ID NO: 2 is the MS-(B1)8 mutant of the MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB: A138P. The variant may comprise one or more of the following substitutions present in MspC: A96G, N102E and A138P. The variant may comprise one or more of the following mutations present in MspD: Deletion of G1, L2V, E5Q, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D. The variant preferably comprises the mutation L88N. A variant of SEQ ID NO: 2 has the mutation L88N in addition to all the mutations of MS-(B1)8 and is called MS-(B2)8. The pore used in the invention is preferably MS-(B2)8. The further preferred variant comprises the mutations G75S/G77S/L88N/Q126R. The variant of SEQ ID NO: 2 has the mutations G75S/G77S/L88N/Q126R in addition to all the mutations of MS-(B1)8 and is called MS-(B2C)8. The pore used in the invention is preferably MS-(B2)8 or MS-(B2C)8.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 7 below. Where amino acids have similar polarity, this can also be determined by reference to the hydropathy scale for amino acid side chains in Table 8.

TABLE 7
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 8
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues 88, 90, 91, 105, 118 and 134 of SEQ ID NO: 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which has its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant has the ability to form a pore. A variant of SEQ ID NO: 2 preferably includes one or more modifications, such as substitutions, additions or deletions, within its α-helices and/or loop regions.

The monomers derived from Msp may be modified to assist their identification or purification, for example by the addition of histidine residues (a hist tag), aspartic acid residues (an asp tag), a streptavidin tag or a flag tag, or by the addition of a signal sequence to promote their secretion from a cell where the polypeptide does not naturally contain such a sequence. An alternative to introducing a genetic tag is to chemically react a tag onto a native or engineered position on the pore. An example of this would be to react a gel-shift reagent to a cysteine engineered on the outside of the pore. This has been demonstrated as a method for separating hemolysin hetero-oligomers (Chem Biol. 1997 July; 4(7):497-505).

The monomer derived from Msp may be labelled with a revealing label. The revealing label may be any suitable label which allows the pore to be detected. Suitable labels are described above.

The monomer derived from Msp may also be produced using D-amino acids. For instance, the monomer derived from Msp may comprise a mixture of L-amino acids and D-amino acids. This is conventional in the art for producing such proteins or peptides.

The monomer derived from Msp contains one or more specific modifications to facilitate nucleotide discrimination. The monomer derived from Msp may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomer derived from Msp can be produced using standard methods known in the art. The monomer derived from Msp may be made synthetically or by recombinant means. For example, the pore may be synthesized by in vitro translation and transcription (IVTT). Suitable methods for producing pores are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

The transmembrane protein pore is also preferably derived from α-hemolysin (α-HL).

In some embodiments, the transmembrane protein pore is chemically modified. The pore can be chemically modified in any way and at any site. The transmembrane protein pore is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein pore may be chemically modified by the attachment of any molecule. For instance, the pore may be chemically modified by attachment of a dye or a fluorophore.

Any number of the monomers in the pore may be chemically modified. One or more, such as 2, 3, 4, 5, 6, 7, 8, 9 or 10, of the monomers is preferably chemically modified as discussed above.

The reactivity of cysteine residues may be enhanced by modification of the adjacent residues. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the pore before a linker is attached.

The molecule (with which the pore is chemically modified) may be attached directly to the pore or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

The helicase or construct may be covalently attached to the pore. The helicase or construct is preferably not covalently attached to the pore. The application of a voltage to the pore and helicase or construct typically results in the formation of a sensor that is capable of sequencing target polynucleotides. This is discussed in more detail below.

Any of the proteins described herein, i.e. the helicases, the transmembrane protein pores or constructs, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the helicase, pore or construct. An example of this would be to react a gel-shift reagent to a cysteine engineered on the outside of the pore. This has been demonstrated as a method for separating hemolysin hetero-oligomers (Chem Biol. 1997 July; 4(7):497-505).

The helicase, pore or construct may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Proteins may be made synthetically or by recombinant means. For example, the helicase, pore or construct may be synthesized by in vitro translation and transcription (IVTT). The amino acid sequence of the helicase, pore or construct may be modified to include non-naturally occurring amino acids or to increase the stability of the protein. When a protein is produced by synthetic means, such amino acids may be introduced during production. The helicase, pore or construct may also be altered following either synthetic or recombinant production.

The helicase, pore or construct may also be produced using D-amino acids. For instance, the pore or construct may comprise a mixture of L-amino acids and D-amino acids. This is conventional in the art for producing such proteins or peptides.

The helicase, pore or construct may also contain other non-specific modifications as long as they do not interfere with pore formation or helicase or construct function. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

The helicase, pore and construct can be produced using standard methods known in the art. Polynucleotide sequences encoding a helicase, pore or construct may be derived and replicated using standard methods in the art. Polynucleotide sequences encoding a helicase, pore or construct may be expressed in a bacterial host cell using standard techniques in the art. The helicase, pore and/or construct may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The helicase, pore and/or construct may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

The method of the invention involves measuring one or more characteristics of the target polynucleotide. The method may involve measuring two, three, four or five or more characteristics of the target polynucleotide. The one or more characteristics are preferably selected from (i) the length of the target polynucleotide, (ii) the identity of the target polynucleotide, (iii) the sequence of the target polynucleotide, (iv) the secondary structure of the target polynucleotide and (v) whether or not the target polynucleotide is modified. Any combination of (i) to (v) may be measured in accordance with the invention.

For (i), the length of the polynucleotide may be measured for example by determining the number of interactions between the target polynucleotide and the pore or the duration of interaction between the target polynucleotide and the pore.

For (ii), the identity of the polynucleotide may be measured in a number of ways. The identity of the polynucleotide may be measured in conjunction with measurement of the sequence of the target polynucleotide or without measurement of the sequence of the target polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the target polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106(19):7702-7, Lieberman K R et al, J Am Chem Soc. 2010; 132(50):17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the target polynucleotide is modified by methylation, by oxidation, by damage, with one or more proteins or with one or more labels, tags or spacers. Specific modifications will result in specific interactions with the pore which can be measured using the methods described below. For instance, methylcytosine may be distinguished from cytosine on the basis of the current flowing through the pore during its interaction with each nucleotide.

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and International Application WO-2000/28312. Alternatively, electrical measurements may be made using a multi-channel system, for example as described in International Application WO-2009/077734 and International Application WO-2011/067559.

In a preferred embodiment, the method comprises:
(a) contacting the target polynucleotide with a transmembrane pore and a helicase of the invention or a construct of the invention such that the target polynucleotide moves through the pore and the helicase or construct controls the movement of the target polynucleotide through the pore; and
(b) measuring the current passing through the pore as the polynucleotide moves with respect to the pore wherein the current is indicative of one or more characteristics of the target polynucleotide and thereby characterising the target polynucleotide.

The methods may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The methods may be carried out using the apparatus described in International Application No. PCT/GB08/000562 (WO 2008/102120).

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp.

The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Examples. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. Hel308, XPD, RecD and TraI helicases surprisingly work under high salt concentrations. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffer include, but are not limited to, HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

The method may be carried out in the presence of free nucleotides or free nucleotide analogues and/or an enzyme cofactor that facilitates the action of the helicase or construct. The method may also be carried out in the absence of free nucleotides or free nucleotide analogues and in the absence of an enzyme cofactor. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the helicase or construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The target polynucleotide may be contacted with the helicase or construct and the pore in any order. In is preferred that, when the target polynucleotide is contacted with the helicase or construct and the pore, the target polynucleotide firstly forms a complex with the helicase or construct. When the voltage is applied across the pore, the target polynucleotide/helicase or construct complex then forms a complex with the pore and controls the movement of the polynucleotide through the pore.

### Other Methods

The invention also provides a method of forming a sensor for characterising a target polynucleotide. The method comprises forming a complex between a pore and a helicase of the invention or a construct of the invention. The helicase may be any of those discussed above. Any number and combination of helicases of the invention discussed above with reference to the series and methods of the invention may be used.

The complex may be formed by contacting the pore and the helicase or construct in the presence of the target polynucleotide and then applying a potential across the pore. The applied potential may be a chemical potential or a voltage potential as described above. Alternatively, the complex may be formed by covalently attaching the pore to the helicase or construct. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target polynucleotide. The method preferably comprises forming a complex between a pore derived from Msp and a helicase of the invention or a construct of the invention. Any of the embodiments discussed above with reference to the methods of the invention equally apply to this method. The invention also provides a sensor produced using the method of the invention.

### Kits

The present invention also provides kits for characterising a target polynucleotide.

In one embodiment, the kit comprises (a) a pore and (b) a helicase of the invention of the invention or a construct of the invention. The pore may be any of those discussed above.

In another embodiment, the kit comprises (a) a helicase of the invention or a construct of the invention and (b) one or more loading moieties. Each loading moiety may be any moiety that is capable of being attached to the target polynucleotide. Each loading moiety may be any length as long as the helicase or construct may bind and it can be attached to the target polynucleotide.

The one or more loading moieties are preferably synthetic or artificial. The one or more loading moieties are preferably non-natural.

Suitable loading moieties include, but are not limited to a polymeric linker, a chemical linker, a polynucleotide or a polypeptide. The one or more loading moieties preferably comprise a polynucleotide or a loading polynucleotide. In such embodiments, the helicase or construct are preferably bound to (or attached to) the polynucleotide. Any of the polynucleotides discussed above may be used. Preferably, the one or more loading moieties comprise DNA, RNA, modified DNA (such as abasic DNA), RNA, PNA, LNA, BNA or PEG. The one or more loading moieties more preferably comprise single stranded or double stranded DNA or RNA.

The one or more loading moieties preferably comprise a single stranded polynucleotide to which the one or more polynucleotide binding proteins are bound (or attached).

At least one of the one or more loading moieties is preferably a Y adaptor. The Y adaptor typically comprises (a) a double stranded region and (b) a single stranded region or a region that is not complementary at the other end. The Y adaptor may be described as having an overhang if it comprises a single stranded region. The presence of a non-complementary region in the Y adaptor gives the adaptor its Y shape since the two strands typically do not hybridise to each other unlike the double stranded portion. The Y adaptor preferably comprises one or more anchors capable of coupling the Y adaptor to a membrane. Anchors are discussed in more detail above. A preferred anchor is cholesterol.

The Y adaptor preferably comprises a leader sequence which preferentially threads into the pore. The leader sequence facilitates the method of the invention. The leader sequence is designed to preferentially thread into the pore and thereby facilitate the movement of target polynucleotide with respect to the pore, such as through the pore. The leader sequence can also be used to link the polynucleotide to the one or more anchors as discussed above.

The leader sequence typically comprises a polymer. The polymer is preferably negatively charged. The polymer is preferably a polynucleotide, such as DNA or RNA, a modified polynucleotide (such as abasic DNA), PNA, LNA, BNA, polyethylene glycol (PEG) or a polypeptide. The leader preferably comprises a polynucleotide and more preferably comprises a single stranded polynucleotide. The leader sequence can comprise any of the polynucleotides discussed above. The single stranded leader sequence most preferably comprises a single strand of DNA, such as a poly dT section. The leader sequence preferably comprises a spacer as discussed below.

The leader sequence can be any length, but is typically 10 to 150 nucleotides in length, such as from 20 to 150 nucleotides in length. The length of the leader typically depends on the transmembrane pore used in the method.

At least one of the one or more loading moieties is preferably a bridging moiety. The bridging moiety is most preferably a hairpin loop or a hairpin loop adaptor. Suitable hairpin loop adaptors can be designed using methods known in the art. The hairpin loop may be any length. If used as a loading moiety, the hairpin loop is typically 400 or fewer nucleotides, such as 350 or fewer nucleotides, 300 or fewer nucleotides, 250 or fewer nucleotides, 200 or fewer nucleotides, 150 or fewer nucleotides, 100 or fewer nucleotides, 90 or fewer nucleotides, 80 or fewer nucleotides, 70 or fewer nucleotides, 60 or fewer nucleotides, 50 or fewer nucleotides, 40 or fewer nucleotides, 30 or fewer nucleotides, 20 or fewer nucleotides or 10 or fewer nucleotides, in length. The hairpin loop is preferably from about 1 to 400, from 2 to 300, from 5 to 200, from 6 to 100 nucleotides in length. Hairpin loops are formed when two complementary parts of a polynucleotide hybridise to form a double stranded sequence (called a stem). If used as a loading moiety, the stem of the hairpin loop is preferably 200 or fewer nucleotide pairs, such as 150 or fewer nucleotide pairs, 100 or fewer nucleotide pairs, 90 or fewer nucleotide pairs, 80 or fewer nucleotide pairs, 70 or fewer nucleotide pairs, 60 or fewer nucleotide pairs, 50 or fewer nucleotide pairs, 40 or fewer nucleotide pairs, 30 or fewer nucleotide pairs, 20 or fewer nucleotide pairs or 10 or fewer nucleotide pairs, in length. The one or more polynucleotide binding proteins typically bind to the loop of the hairpin, i.e. not the stem.

If the target polynucleotide is double stranded, the one or more loading moieties preferably comprise a Y adaptor and optionally a bridging moiety, such as a hairpin loop adaptor. If at least one or more of the loading moieties is Y adaptor, it may be used in combination with a bridging adaptor that does not have any polynucleotide binding proteins bound or attached.

The helicase or construct may be stalled at one or more spacers on the one or more loading moieties. Spacers are defined in PCT/GB2014/050175 (WO 2014/135838). Preferred spacers include, but are not limited to, nitroindoles, 5-nitroindoles, inosines, acridines, 2-aminopurines, 2-6-diaminopurines, 5-bromo-deoxyuridines, inverted thymidines (inverted dTs), inverted dideoxy-thymidines (ddTs), dideoxy-cytidines (ddCs), 5-methylcytidines, 5-hydroxymethylcytidines, 2′-O-Methyl RNA bases, Iso-deoxycytidines (Iso-dCs), Iso-deoxyguanosines (Iso-dGs), iSpC3 groups (i.e. nucleotides which lack sugar and a base), photo-cleavable (PC) groups, hexandiol groups, spacer 9 (iSp9) groups, spacer 18 (iSp18) groups, a polymer or thiol connections. The spacer may comprise any combination of these groups. Many of these groups are commercially available from IDT® (Integrated DNA Technologies@).

Any number of one or more loading moieties may be used. The method may comprise attaching two or more loading moieties each having a helicase or construct bound (attached) thereto. For instance, a loading moiety may be attached to each end of the target polynucleotide. In such embodiments, one loading moiety is preferably a Y adaptor and the other loading moiety may be a bridging moiety, such as a hairpin loop adaptor.

The one or more loading moieties may be attached to the target polynucleotide in any manner. The one or more loading moieties are preferably covalently attached to the target polynucleotide.

The one or more loading moieties are most preferably ligated to the target polynucleotide. The one or more loading moieties may be ligated to either end of the polynucleotide, i.e. the 5′ or the 3′ end. Loading moieties may be ligated to both ends of the target polynucleotide. The one or more loading moieties may be ligated to the polynucleotide using any method known in the art. The one or more loading moieties may be ligated to the polynucleotide in the absence of ATP or using gamma-S-ATP (ATPγS) instead of ATP.

The one or more loading moieties may be ligated using a ligase, such as T4 DNA ligase, E. coli DNA ligase, Taq DNA ligase, Tma DNA ligase and 9°N DNA ligase. The ligase is preferably used under the conditions set out in Example 3.

The helicase or construct preferably remains bound (attached) to the loading moiety once the loading moiety has been attached to the target polynucleotide. After it has been attached in accordance with the invention, the helicase or construct may unbind from the one or more loading moieties.

Any of the embodiments discussed above with reference to the method of the invention equally apply to the kits. The helicase may be any of those discussed. The kit may comprise any number and combination of helicases of the invention discussed above with reference to the series and methods of the invention.

The kit may further comprise the components of a membrane, such as the phospholipids needed to form an amphiphilic layer, such as a lipid bilayer.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

### Apparatus

The invention also provides an apparatus for characterising a target polynucleotide. The apparatus comprises a plurality of pores and a plurality of helicases of the invention or a plurality of constructs of the invention. The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention. The helicase may be any of those discussed above with reference to the constructs of the invention, including the helicases of the invention and helicases which are not modified in accordance with the invention. The apparatus may comprise any number and combination of helicases of the invention.

The apparatus is preferably set up to carry out the method of the invention.

The apparatus preferably comprises:
a sensor device that is capable of supporting the plurality of pores and membranes and being operable to perform polynucleotide characterisation using the pores and membranes; and
at least one port for delivery of the material for performing the characterisation.

Alternatively, the apparatus preferably comprises:
a sensor device that is capable of supporting the plurality of pores and membranes being operable to perform polynucleotide characterisation using the pores and membranes; and
at least one reservoir for holding material for performing the characterisation.

The apparatus more preferably comprises:
a sensor device that is capable of supporting the membrane and plurality of pores and membranes and being operable to perform polynucleotide characterising using the pores and membranes;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device. The apparatus may be any of those described in International Application No. PCT/GB08/004127 (published as WO 2009/077734), PCT/GB10/000789 (published as WO 2010/122293), International Application No. PCT/GB10/002206 (published as WO 2011/067559) or International Application No. PCT/US99/25679 (published as WO 00/28312).

### Methods of Producing Helicases of the Invention

The invention also provides methods of producing a modified helicase of the invention. The method comprises providing a Dda helicase and modifying the helicase to form a modified helicase of the invention.

The method preferably further comprises determining whether or not the helicase is capable of controlling the movement of a polynucleotide. Assays for doing this are described above. If the movement of a polynucleotide can be controlled, the helicase has been modified correctly and a helicase of the invention has been produced. If the movement of a polynucleotide cannot be controlled, a helicase of the invention has not been produced.

### Methods of Producing Constructs of the Invention

The invention also provides a method of producing a construct of the invention. The method comprises attaching, preferably covalently attaching, a helicase of the invention to an additional polynucleotide binding moiety. Any of the helicases and moieties discussed above can be used in the methods. The site of and method of covalent attachment are selected as discussed above.

The method preferably further comprises determining whether or not the construct is capable of controlling the movement of a polynucleotide. Assays for doing this are described above. If the movement of a polynucleotide can be controlled, the helicase and moiety have been attached correctly and a construct of the invention has been produced. If the movement of a polynucleotide cannot be controlled, a construct of the invention has not been produced.

The following Examples illustrate the invention.

### EXAMPLES

### Example 1

This example describes the simulations which were run to investigate the interaction between MspA—(G75S/G77S/L88N/D90N/D91N/D118R/Q126R/D134R/E139K)8 (SEQ ID NO: 2 with mutations G75S/G77S/L88N/D90N/D91N/D118R/Q126R/D134R/E139K=MspA mutant 1) or MspA—((Del-L74/G75/D118/L119)D56N/E59R/L88N/D90N/D91N/Q126R/D134R/E139K)8 (SEQ ID NO: 2 with mutations D56N/E59R/L88N/D90N/D91N/Q126R/D134R/E139K and deletion of the amino acids L74/G75/D118/L119=MspA mutant 2) with T4 Dda—E94C/A360C/C109A/C136A (SEQ ID NO: 8 with mutations E94C/A360C/C114A/C171A/C421D)=enzyme mutant 1a).

Simulations were performed using the GROMACS package version 4.0.5, with the GROMOS 53a6 forcefield and the SPC water model.

The MspA mutant 1 and MspA mutant 2 models were based on the crystal structure of MspA found in the protein data bank, accession code 1UUN. The relevant mutations were made using PyMOL, and in the case of MspA mutant 2 the residues L74/G75/D118/L119 were deleted from the barrel. The resultant pore models were then energy minimised using the steepest descents algorithm. The enzyme mutant 1a model was based on the Dda1993 structure found in the protein data bank, accession code 3UPU. Again, relevant mutations were made using PyMOL, and the model was energy minimised using the steepest descents algorithm.

The enzyme mutant 1a model was then placed above MspA mutant 1 and MspA mutant 2. Three simulations were performed for the enzyme mutant 1a/MspA mutant 1 and enzyme mutant 1a/MspA mutant 2 systems, with the orientation of enzyme mutant 1a differing in each simulation (See FIG. 1 for cartoon representations of the three different simulation orientations). The pore was placed into a lipid membrane comprising DPPC molecules and the simulation box was solvated. Throughout the simulation, restraints were applied to the backbone of the pore. However, the enzyme was unrestrained. The system was simulated in the NPT ensemble for 40 ns, using the Berendsen thermostat and Berendsen barostat to 300 K.

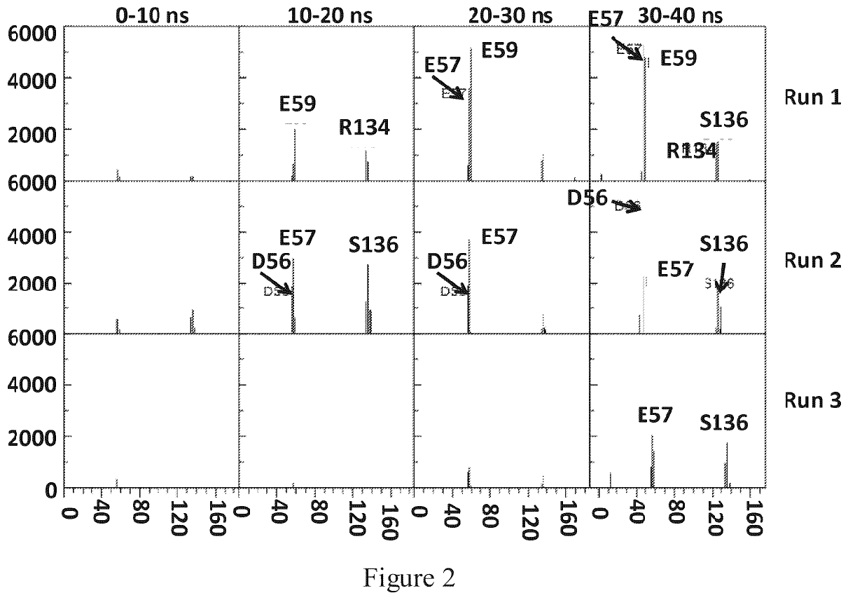

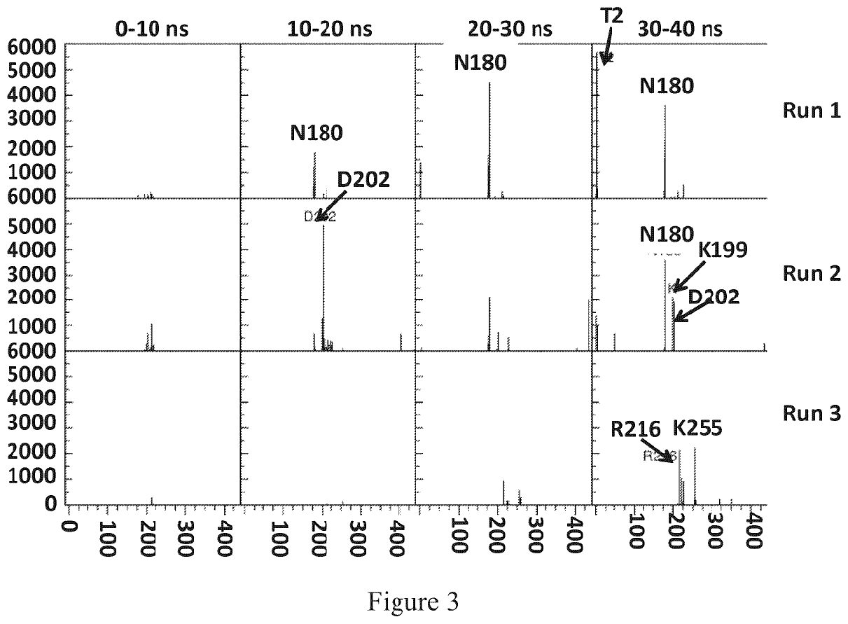

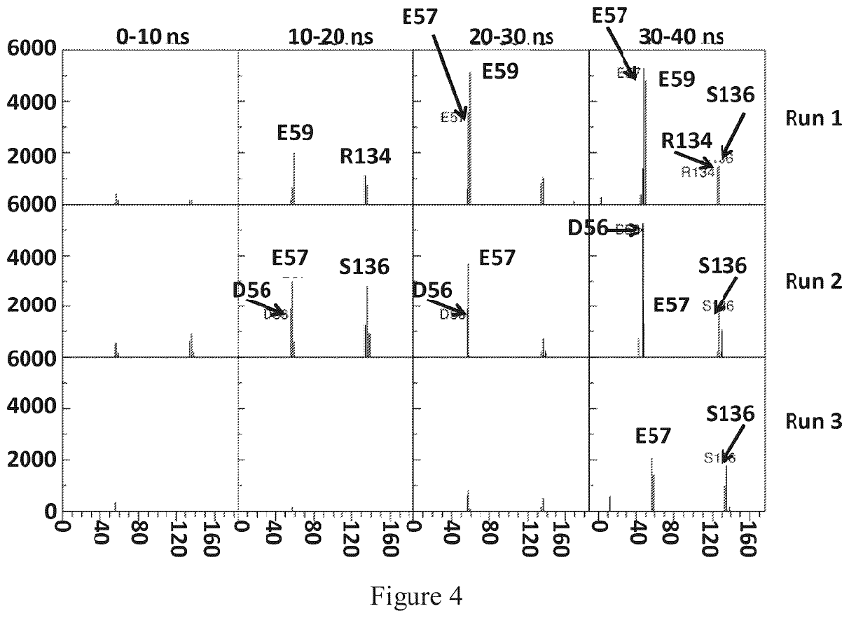

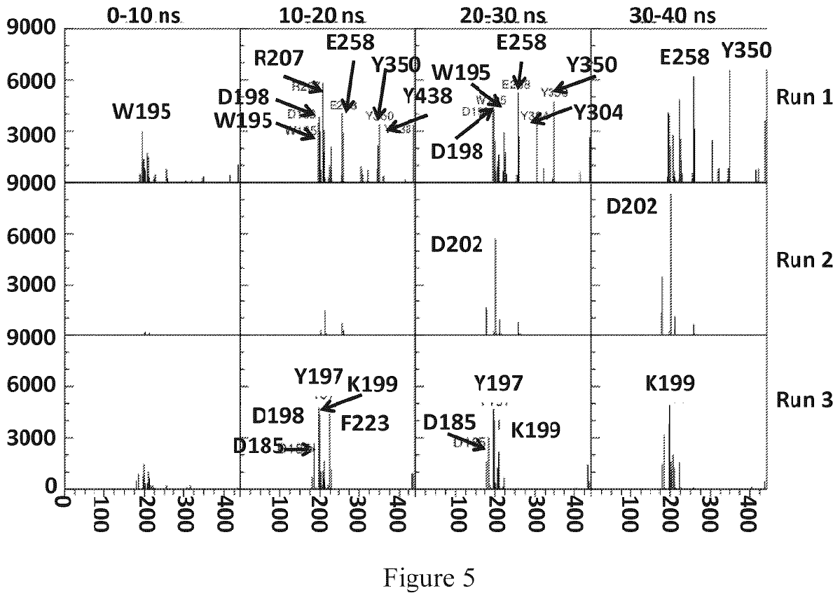

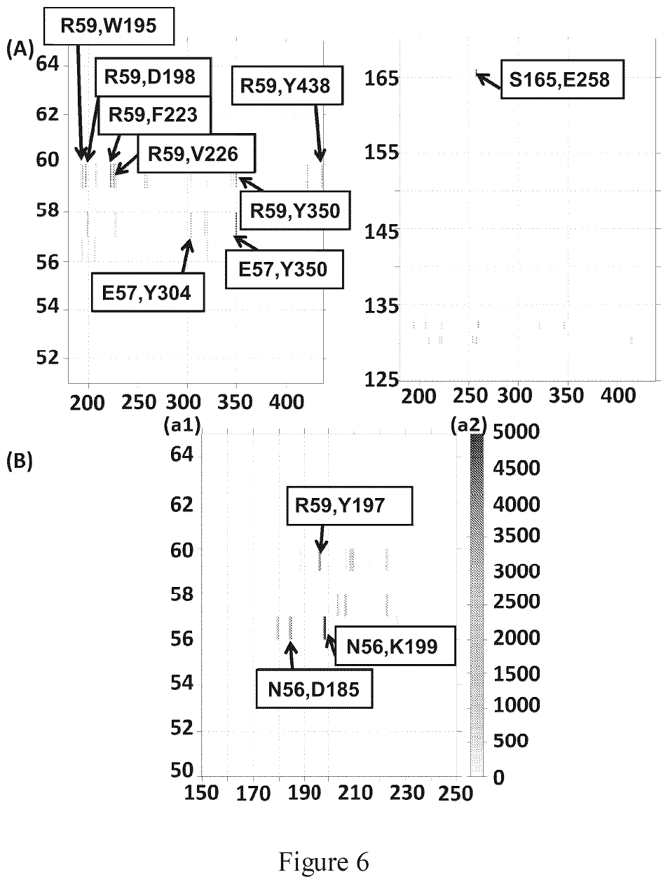

The contacts between the enzyme and pore were analysed using both GROMACS analysis software and also locally written code. FIGS. 2 to 5 showed the amino acid residues which interacted in MspA mutant 1 (FIGS. 2 and 3) and MspA mutant 2 (FIGS. 4 and 5) with the enzyme mutant 1a. The tables below show the amino acid positions in both the pore and the enzyme which were found to interact (Table 9 shows the MspA mutant 1 amino acid contact points observed when the interactions were measured between MspA mutant 1 and enzyme mutant 1a, Table 10 shows the enzyme mutant 1a amino acid contact points observed when the interactions were measured between MspA mutant 1 and enzyme mutant 1a, Table 11 shows the MspA mutant 2 amino acid contact points observed when the interactions were measured between MspA mutant 2 and enzyme mutant 1a, Table 12 shows the enzyme mutant 1a amino acid contact points observed when the interactions were measured between MspA mutant 2 and enzyme mutant 1a). FIG. 6 shows which amino acids in the pore (MspA mutant 2) interacted with particular amino acids in the enzyme (enzyme mutant 1a). The simulation data can be used to identify parts of enzyme mutant 1a which could be modified in order to improve the interaction between the enzyme and the nanopore in order to provide more consistent movement of the target polynucleotide with respect to, such as through, the transmembrane pore.

TABLE 9

| Run 1 | Run 2 | Run 3 |
|---|---|---|
| Pore | Pore | Pore |
| Amino Acid | Amino Acid | Amino Acid |
| Residue | Residue | Residue |
| 57 | 56 | 57 |
| 59 | 57 | 136 |
| 136 | 136 | 59 |
| 134 | 139 | 134 |
| 56 | 52 | 56 |
| 54 | 134 | 12 |
| 12 | 138 | 139 |
| 169 | 55 | 58 |
| 14 | 59 | 137 |
| 58 | | 14 |
| 55 | | 48 |
| 52 | | 169 |
| 138 | | |
| 139 | | |
| 137 | | |

TABLE 10

| Run 1 | Run 2 | Run 3 |
|---|---|---|
| Enzyme | Enzyme | Enzyme |
| Amino Acid | Amino Acid | Amino Acid |
| Residue | Residue | Residue |
| 2 | 180 | 255 |
| 180 | 199 | 216 |
| 179 | 202 | 221 |
| 178 | 1 | 227 |
| 227 | 4 | 351 |
| 4 | 51 | 321 |
| 177 | 434 | 254 |
| 212 | 179 | 258 |
| 1 | 178 | 224 |
| 194 | 177 | 257 |
| 204 | 197 | 256 |
| 176 | 5 | 223 |
| 213 | 201 | 212 |
| 3 | 181 | 308 |
| 216 | 200 | 207 |
| 211 | 6 | 350 |
| 202 | | 228 |
| 224 | | 210 |
| 223 | | 319 |
| 191 | | 304 |
| 199 | | 209 |
| 201 | | 347 |
| 434 | | 261 |
| 405 | | 260 |
| 255 | | 247 |

TABLE 11

| Run 1 | Run 2 | Run 3 |
|---|---|---|
| Pore | Pore | Pore |
| Amino Acid | Amino Acid | Amino Acid |
| Residue | Residue | Residue |
| 59 | 59 | 56 |
| 57 | 57 | 59 |
| 134 | 169 | 57 |
| 136 | 134 | 136 |
| 169 | 136 | 12 |
| 56 | 56 | 14 |
| 137 | 54 | 134 |
| 58 | 14 | 54 |
| 14 | 12 | 169 |
| 135 | | 53 |
| 60 | | |
| 170 | | |

TABLE 12

| Run 1 | Run 2 | Run 3 |
|---|---|---|
| Enzyme | Enzyme | Enzyme |
| Amino Acid | Amino Acid | Amino Acid |
| Residue | Residue | Residue |
| 350 | 202 | 199 |
| 258 | 180 | 197 |
| 223 | 179 | 185 |
| 195 | 212 | 198 |
| 198 | 258 | 207 |
| 438 | 211 | 202 |
| 260 | 198 | 223 |
| 207 | 265 | 180 |
| 226 | 260 | 209 |
| 304 | 259 | 210 |
| 200 | 255 | 203 |
| 227 | 1 | 204 |
| 347 | 200 | 437 |
| 321 | 300 | 200 |
| 422 | 203 | 211 |
| 318 | 261 | 405 |
| 415 | 216 | 227 |
| 210 | 177 | 258 |
| 229 | 213 | 212 |
| 255 | 207 | 256 |
| 224 | 337 | 216 |
| 228 | 204 | 189 |
| 208 | 434 | 228 |
| 193 | 298 | 220 |
| 256 | | 219 |

### Example 2

This example describes the simulations which were run to investigate the interaction between two different enzymes (wild-type Dda 1993 (SEQ ID NO: 8)) and T4 Dda-E94C/A360C (SEQ ID NO: 8 with mutations E94C/A360C)=enzyme mutant 18) and a polynucleotide.

Simulations were performed to assess which residues made contact with the DNA that was within the enzyme binding site. The simulations were performed using the GROMACS package version 4.0.5, with the AMBER-99SB force field and the TIP3P water model.

Two enzymes were simulated, wild-type Dda1993 and enzyme mutant 18. Enzyme mutant 18 was simulated in its closed-complex form, such that a disulphide bond was present between E94C and A360C. The initial structure of wild-type Dda1993 was based on the structure available in the protein data bank, with accession code 3UPU. The structure in this PDB file is Dda1993-K38A. Hence, in the wild-type Dda1993 simulations, residue 38 was mutated back to lysine using PyMOL. The enzyme mutant 18 model was also based on the structure in 3UPU, with the relevant mutations made in PyMOL. The DNA simulated in both enzyme simulations was the DNA present in the crystal structure of 3UPU (DNA sequence is poly(dT)). The resultant enzyme/DNA models were then energy minimised using the steepest descents algorithm. The simulation box was then solvated and another round of energy minimisation was performed. Throughout the simulation the enzyme and DNA were unrestrained. The system was simulated in the NPT ensemble for 20 ns, using the Berendsen thermostat and Berendsen barostat to 300 K.

The contacts between the enzyme and DNA were analysed using both GROMACS analysis software and also locally written code. The tables below show the amino acids in the two enzymes which interacted with the DNA present in the crystal structure 3UPU (Tables 13 and 14 show the wild-type Dda 1993 amino acid contact points observed when the interactions were measured between wild-type Dda1993 and the DNA and Tables 15 and 16 show enzyme mutant 18 amino acid contact points observed when the interactions were measured between enzyme mutant 18 and the DNA). The simulation data can be used to identify positions in the Dda1993 enzyme and enzyme mutant 18 which could be mutated in order to improve the interaction between the enzyme and the DNA in order to provide more consistent movement of the target polynucleotide with respect to, such as through, the transmembrane pore.

TABLE 13

Amino Acid
Residue WT
Dda1993
N242
K397
H396
N293
H82
H64
F98
V150
Y415
T63
F240
T241
T394
T80
F276
P89
N88
S83
I289
P152
P274

TABLE 14

Amino Acid
Residue WT
Dda1993
I87
D417
P62
S287
H414
K243
M119
A416
L420
K86
N292
V96
D151
N155
W378
E288
T278
V286
K123
R148
L97

TABLE 15

Amino Acid
Residue Enzyme
Mutant 18
H82
K397
N242
H64
V150
H396
T241
N293
T63
N88
F98
F240
T80
S83
P89
T394
S287
F276
Y415
I87
D417

TABLE 16

Amino Acid
Residue Enzyme
Mutant 18
V286
I289
P62
N292
R148
K101
K243
A416
K86
L420
N155
E288
P274
D151
P152
P285
M119
T278
D121
K284
Q272

### Example 3

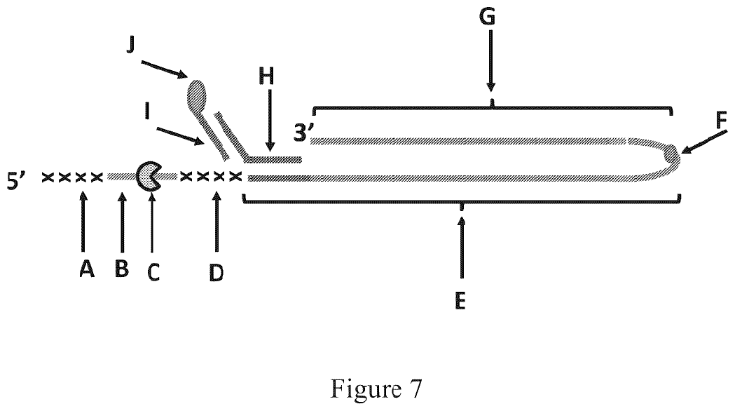

This example compares movement control of DNA construct X (see FIG. 7) through a nanopore using T4 Dda-E94C/C109A/C136A/A360C (SEQ ID NO: 8 with mutations E94C/C109A/C136A/A360C and then (ΔM1)G1)) (Enzyme mutant 1) with a number of different helicases. All of the helicases tested controlled the movement of the DNA through the nanopore and changes in current as the DNA translocated through the nanopore were observed. The helicases tested had either a) at least one amino acid substitution which interacted with one or more nucleotides in single stranded DNA (ssDNA) or b) one or more modifications in the part of the helicase which interacted with the transmembrane pore or both changes a) and b). This example investigates the number of complement slips forward per 3.6 kb, number of complement slips forward per kb, the % of bases missed in construct X due to slipping forward, total length of slips forward in the complement and average length of slip forward. The helicases investigated in the example moved along the polynucleotide in a 5′ to 3′ direction. When the 5′end of the polynucleotide (the end away from which the helicase moves) was captured by the pore, the helicase worked with the direction of the field resulting from the applied potential and moved the threaded polynucleotide into the pore and into the trans chamber. In this Example, slipping forward involved the DNA moving forwards relative to the pore (i.e. towards its 3′ and away from it 5′ end) at least 4 consecutive nucleotides.

### Materials and Methods

Prior to setting up the experiment, DNA construct X (final concentration 0.1 nM) was pre-incubated at room temperature for five minutes with the appropriate enzyme (see list of enzymes provided below (final concentration added to the nanopore system 10 nM, which was provided in buffer (253 mM KCl, 50 mM potassium phosphate, pH 8.0, 2 mM EDTA)). After five minutes, TMAD (100 μM final concentration added to the nanopore system) was added to the pre-mix and the mixture incubated for a further 5 minutes. Finally, MgCl2 (2 mM final concentration added to the nanopore system), ATP (2 mM final concentration added to the nanopore system) and KCl (500 mM final concentration added to the nanopore system) were added to the pre-mix.

Electrical measurements were acquired from single MspA nanopores (MspA—((Del-L74/G75/D118/L119)D56F/E59R/L88N/D90N/D91N/Q126R/D134R/E139K)8 (SEQ ID NO: 2 with mutations D56F/E59R/L88N/D90N/D91N/Q126R/D134R/E139K and deletion of the amino acids L74/G75/D118/L119) (MspA mutant 3)) inserted in block co-polymer in buffer (25 mM K Phosphate buffer, 150 mM Potassium Ferrocyanide (II), 150 mM Potassium Ferricyanide (III), pH 8.0). After achieving a single pore inserted in the block co-polymer, then buffer (2 mL, 25 mM K Phosphate buffer, 150 mM Potassium Ferrocyanide (II), 150 mM Potassium Ferricyanide (III), pH 8.0) was flowed through the system to remove any excess MspA nanopores. 150 uL of 500 mM KCl, 25 mM K Phosphate, pH8.0 was then flowed through the system. After 10 minutes a further 150 uL of 500 mM KCl, 25 mM K Phosphate, pH8.0 was flowed through the system and then the enzyme (see list below, 10 nM final concentration), DNA construct X (0.1 nM final concentration), fuel (MgCl2 2 mM final concentration, ATP 2 mM final concentration) pre-mix (150 μL total) was then flowed into the single nanopore experimental system. The experiment was run at −140 mV and helicase-controlled DNA movement monitored.

### Results

A number of different helicases were investigated in order to determine the effect of at least one or more substitutions to regions of the helicase which were thought to interact with the DNA construct X or one or more modifications which were thought to interact with the nanopore. Five different parameters were investigated in order to identify helicases which exhibited improved helicase controlled DNA translocation 1) the number of complement slips forward per 3.6 kb, 2) number of complement slips forward per kb, 3) the % of bases missed in construct X due to slipping forward, 4) total length of slips forward in the complement and 5) average length of slip forward.

The measurement of slips forward per kilobase or per 3.6 kb were calculated using the following procedure 1) the helicase controlled DNA movements were mapped to a model using an HMM algorithm, 2) the helicase-controlled DNA movements were then subjected to filtering, 3) the mapped helicase controlled DNA movements were checked to ensure accurate mapping, 4) the transitions that were classified as a slipping forward movement of at least four consecutive nucleotides were determined per kilobase or per 3.6 kb. The % bases missed in construct X due to slipping forward is a measure of the number of bases in construct X which are missed as a result of slips forward along DNA construct X expressed as a percentage. The total length of complement slips is the sum of all slips in the complement section of the strand. Average slip length is the sum of all slips in the complement section of the strand divided by the total number of slips in the complement.

Table 17 below shows the different enzymes tested which were compared to enzyme mutant 1. Of the enzymes tested, mutants 2 to 13 and 15 to 17 exhibited an improvement in at least one of the parameters 1 to 5 when compared to enzyme mutant 1.

Mutants 5 to 13 have at least one amino acid, which interacted with the sugar and/or base of one or more nucleotides in single stranded DNA (ssDNA), substituted with an amino acid which comprised a larger side chain (R group) and had no one or more modifications in the part of the helicase which interacted with the transmembrane pore. All of mutants 5 to 13 exhibited an improvement in at least one of parameters 1 to 5 when compared with enzyme mutant 1 which was attributed to the amino acid substitution which comprised a larger side chain (R group) and which interacted with the sugar and/or base of one or more nucleotides in single stranded DNA (ssDNA). Clearly, making at least one substitution with a larger side chain group at positions which interacted with the sugar and or base of one or more nucleotides in the single stranded (ssDNA) resulted in improved movement control.

Mutant 14 has at least one amino acid, which interacted with the sugar and/or base of one or more nucleotides in single stranded DNA (ssDNA), substituted with an amino acid which comprised a smaller side chain (R group) and had no one or more modifications in the part of the helicase which interacted with the transmembrane pore. Mutant 14 exhibited no improvement in any of parameters 1 to 5 when compared with enzyme mutant 1 which was attributed to the amino acid substitution which comprised a smaller side chain (R group) and which interacted with the sugar and/or base of one or more nucleotides in single stranded (ssDNA). Clearly, making at least one substitution with a smaller side chain groups at positions which interacted with the sugar and or base of one or more nucleotides in single stranded DNA (ssDNA) resulted in poorer movement control.

Mutant 4 has at least one amino acid substitution which interacted with one or more phosphate groups in one or more nucleotides in single stranded DNA (ssDNA), at least one amino acid, which interacted with the sugar and/or base of one or more nucleotides in single stranded DNA (ssDNA), substituted with an amino acid which comprised a larger side chain (R group) and had no one or more modifications in the part of the helicase which interacted with the transmembrane pore. Mutant 4 exhibited an improvement in at least one of parameters 1 to 5 when compared with enzyme mutant 1 which was attributed to the combination of amino acid substitutions e.g. one which interacted with one or more phosphate groups in one or more nucleotides in single stranded DNA (ssDNA) and the second which interacted with the sugar and/or base of one or more nucleotides in single stranded DNA (ssDNA). Furthermore, Mutant 4 exhibited an improvement in at least one of parameters 1 to 5 when compared with enzyme mutant 9 which was attributed to the amino acid substitution which interacted with one or more phosphate groups in one or more nucleotides in single stranded DNA (ssDNA). Clearly, making substitutions with larger side chain groups at positions which interacted with the sugar and/or base of one or more nucleotides and making substitutions which interacted with one or more phosphate groups in one or more nucleotides in single stranded DNA (ssDNA) resulted in improved movement control.

Mutant 2 had at least one amino acid, which interacted with the sugar and/or base of one or more nucleotides in single stranded DNA (ssDNA), substituted with an amino acid which comprised a larger side chain (R group) and had one or more modifications in the part of the helicase which interacted with the transmembrane pore. Mutant 2 exhibited an improvement in at least one of parameters 1 to 5 when compared with enzyme mutant 1 which was attributed to the combination of changes e.g. the first at least one substitution which interacted with the sugar and/or base of one or more nucleotides in single stranded DNA (ssDNA) and the second one or more modifications in the part of the helicase which interacted with the transmembrane pore. Mutant 2 also exhibited an improvement in at least one of parameters 1 to 5 when compared with enzyme mutant 9 which was attributed to the second one or more modifications in the part of the helicase which interacted with the transmembrane pore. Furthermore, mutant 2 exhibited an improvement in at least one of parameters 1 to 5 when compared with enzyme mutant 16 which was attributed to the first at least one substitution which interacted with the sugar and/or base of one or more nucleotides in single stranded DNA (ssDNA). Clearly, making the combination of changes (the first at least one substitution which interacted with the sugar and/or base of one or more nucleotides in single stranded DNA (ssDNA) and the second one or more modifications in the part of the helicase which interacted with the transmembrane pore) resulted in an enzyme which exhibited improved movement control.

Mutant 3 had at least one amino acid, which interacted with the sugar and/or base of one or more nucleotides in single stranded DNA (ssDNA), substituted with an amino acid which comprised a larger side chain (R group) and had one or more modifications in the part of the helicase which interacted with the transmembrane pore. Mutant 3 exhibited an improvement in at least one of parameters 1 to 5 when compared with enzyme mutant 1 which was attributed to the combination of changes e.g. the first at least one substitution which interacted with the sugar and/or base of one or more nucleotides in single stranded DNA (ssDNA) and the second one or more modifications in the part of the helicase which interacted with the transmembrane pore. Mutant 3 also exhibited an improvement in at least one of parameters 1 to 5 when compared with enzyme mutant 9 which was attributed to the second one or more modifications in the part of the helicase which interacted with the transmembrane pore. Furthermore, mutant 3 exhibited an improvement in at least one of parameters 1 to 5 when compared with enzyme mutant 17 which was attributed to the first at least one substitution which interacted with the sugar and/or base of one or more nucleotides in single stranded DNA (ssDNA). Clearly, making the combination of substitutions (the first at least one substitution which interacted with the sugar and/or base of one or more nucleotides in single stranded DNA (ssDNA) and the second one or more modifications in the part of the helicase which interacted with the transmembrane pore) resulted in an enzyme which exhibited improved movement control.

### Enzyme ID's

Enzyme mutant 1 = T4 Dda-E94C/C109A/C136A/A360C (SEQ ID NO: 8 with mutations
E94C/C109A/C136A/A360C and then (ΔM1)G1))
Enzyme mutant 2 = T4 Dda-E94C/F98W/C109A/C136A/K199L/A360C (SEQ ID NO: 8 with
mutations E94C/F98W/C109A/C136A/K199L/A360C and then (ΔM1)G1))
Enzyme mutant 3 = T4 Dda- F98W/E94C/C109A/C136A/K194L/A360C (SEQ ID NO: 8 with
mutations F98W/E94C/C109A/C136A/K194L/A360C and then (ΔM1)G1))
Enzyme mutant 4 = T4 Dda-S83H/E94C/F98W/C109A/C136A/A360C (SEQ ID NO: 8 with
mutations S83H/E94C/F98W/C109A/C136A/A360C and then (ΔM1)G1))
Enzyme mutant 5 = T4 Dda-E94C/F98W/C109A/C136A/F276K/A360C (SEQ ID NO: 8 with
mutations E94C/F98W/C109A/C136A/F276K/A360C and then (ΔM1)G1))
Enzyme mutant 6 = T4 Dda-E94C/F98W/C109A/C136A/S287R/A360C (SEQ ID NO: 8 with
mutations E94C/F98W/C109A/C136A/S287R/A360C and then (ΔM1)G1))
Enzyme mutant 7 = T4 Dda-E94C/F98W/C109A/C136A/S287W/A360C (SEQ ID NO: 8 with
mutations E94C/F98W/C109A/C136A/S287W/A360C and then (ΔM1)G1))
Enzyme mutant 8 = T4 Dda-E94C/F98W/C109A/C136A/S287F/A360C (SEQ ID NO: 8 with
mutations E94C/F98W/C109A/C136A/S287F/A360C and then (ΔM1)G1))
Enzyme mutant 9 = T4 Dda-E94C/F98W/C109A/C136A/A360C (SEQ ID NO: 8 with mutations
E94C/F98W/C109A/C136A/A360C and then (ΔM1)G1))
Enzyme mutant 10 = T4 Dda-P89F/E94C/C109A/C136A/A360C (SEQ ID NO: 8 with mutations
P89F/E94C/C109A/C136A/A360C and then (ΔM1)G1))
Enzyme mutant 11 = T4 Dda-E94C/C109A/C136A/V150H/A360C (SEQ ID NO: 8 with
mutations /C109A/C136A/V150H/A360C and then (ΔM1)G1))
Enzyme mutant 12 = T4 Dda-E94C/C109A/C136A/V150I/A360C (SEQ ID NO: 8 with
mutations E94C/C109A/C136A/V150I/A360C and then (ΔM1)G1))
Enzyme mutant 13 = T4 Dda-E94C/C109A/C136A/P152F/A360C (SEQ ID NO: 8 with
mutations E94C/C109A/C136A/P152F/A360C and then (ΔM1)G1))
Enzyme mutant 14 = T4 Dda-E94C/F98A/C109A/C136A/A360C (SEQ ID NO: 8 with
mutations E94C/F98A/C109A/C136A/A360C and then (ΔM1)G1))
Enzyme mutant 15 = T4 Dda-E94C/C109A/C136A/K199L/A360C (SEQ ID NO: 8 with
mutations E94C/C109A/C136A/K199L/A360C and then (ΔM1)G1))
Enzyme mutant 16 = T4 Dda-E94C/C109A/C136A/K194L/A360C (SEQ ID NO: 8 with
mutations E94C/C109A/C136A/K194L/A360C and then (ΔM1)G1))
Enzyme mutant 17 = T4 Dda-E94C/C109A/C136A/W195A/A360C (SEQ ID NO: 8 with
mutations E94C/C109A/C136A/W195A/A360C and then (ΔM1)G1))

TABLE 17

| | | | % complement | | |
|---|---|---|---|---|---|
| | Number complement | Number complement | bases missed | Total length of | Average |
| Enzyme | slips per 3.6 kb | slips per kb | through slipping | complement slips | slip length |
| 1 | 16 | 4.4 | 10.5 | 377 | 23.6 |
| 2 | 6 | 1.7 | 3.6 | 129 | 21.5 |
| 3 | 3 | 0.8 | 1.9 | 70 | 23.3 |
| 4 | 6 | 1.7 | 3.4 | 121 | 24 |
| 5 | 7.7 | 2.1 | 3.9 | 140 | 22 |
| 6 | 8.1 | 2.3 | 4.4 | 158 | 27 |
| 7 | 5.8 | 1.6 | 3.1 | 112 | 27 |
| 8 | 6.7 | 1.9 | 3.7 | 134 | 28 |
| 9 | 11 | 3.1 | 6.5 | 233 | 21.2 |
| 10 | 17 | 4.7 | 10.3 | 372 | 21.9 |
| 11 | 6 | 1.7 | 3.7 | 134 | 22.3 |
| 12 | 12 | 3.3 | 8.7 | 314 | 26.2 |
| 13 | 12 | 3.3 | 9.9 | 355 | 29.6 |
| 14 | 17 | 4.7 | 11.7 | 420 | 24.7 |
| 15 | 8 | 2.2 | 5.3 | 191 | 23.9 |
| 16 | 4 | 1.1 | 2.8 | 100 | 25.0 |
| 17 | 15 | 4.2 | 10.3 | 371 | 24.7 |

### Example 4

This example shows how the helicase T4 Dda—E94C/F98W/C109A/C136A/K194L/A360C (SEQ ID NO: 8 with mutations E94C/F98W/C109A/C136A/K194L/A360C and then (ΔM1)G1)) controlled the movement of DNA construct X (see FIG. 7) through a CsgG nanopore (CsgG-Eco-(Y51T/F56Q)-StrepII(C))9 (SEQ ID NO: 66 with mutations Y51T/F56Q where StepII(C) is SEQ ID NO: 67 and is attached at the C-terminus).

### Materials and Methods

DNA construct X helicase (T4 Dda—E94C/F98W/C109A/C136A/K194L/A360C (SEQ ID NO: 8 with mutations E94C/F98W/C109A/C136A/K194L/A360C and then (ΔM1)G1)) pre-mix was prepared as described in the materials and methods section of Example 3.

Electrical measurements were acquired from single CsgG nanopores (CsgG-Eco-(Y51T/F56Q)-StrepII(C))9 (SEQ ID NO: 66 with mutations Y51T/F56Q where StepII(C) is SEQ ID NO: 67 and is attached at the C-terminus) inserted in block co-polymer in a similar method as described in Example 3 except the nanopore was CsgG and not MspA.

### Results

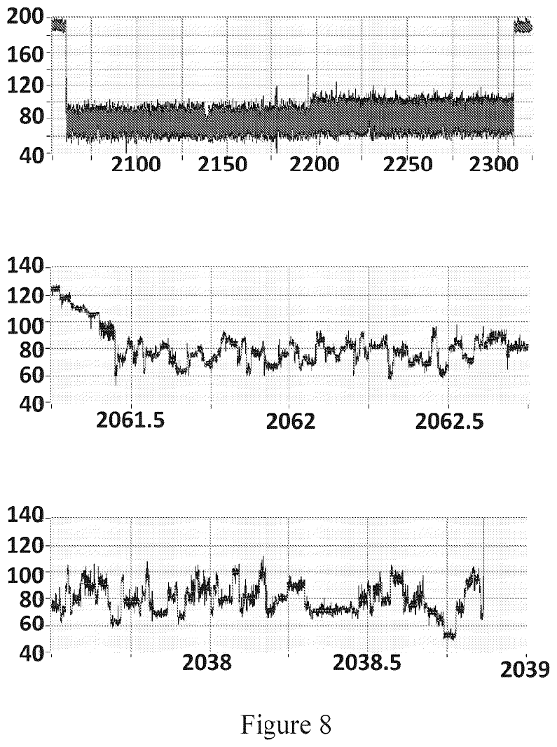

Helicase controlled DNA movement was observed as T4 Dda—E94C/F98W/C109A/C136A/K194L/A360C (SEQ ID NO: 8 with mutations E94C/F98W/C109A/C136A/K194L/A360C and then (ΔM1)G1)) controlled the movement of DNA construct X (see FIG. 7) through a CsgG nanopore (CsgG-Eco-(Y51T/F56Q)-StrepII(C))9 (SEQ ID NO: 66 with mutations Y51T/F56Q where StepII(C) is SEQ ID NO: 67 and is attached at the C-terminus). An example of a current trace of a helicase controlled DNA movement is shown in FIG. 8A and zoomed in views of the same trace are shown in FIGS. 8B and C.

### Example 5

This example shows how a hairpin was attached to the 3′ end of an RNA strand and the RNA strand was reverse transcribed to create an RNA/DNA hybrid. Subsequently a non-RNA polynucleotide was attached to the 5′ end of the RNA strand in the RNA/DNA hybrid to facilitate loading of a DNA helicase, T4 Dda (E94C/F98W/C109A/C136A/K194L/A360C (SEQ ID NO: 8 with mutations E94C/F98W/C109A/C136A/K194L/A360C and then (ΔM1)G1)). Helicase-controlled movement of the RNA/DNA construct through a nanopore was observed.

### Materials and Methods

### 1. Hairpin Ligation

The reagents listed in Table 18 below were mixed and placed on a thermocycler set to the program in Table 19 below. The mixture was then purified using Agencourt Ampure SPRI beads at a ratio of 1.8 μL SPRI beads per μL of sample. After purification, reverse transcription was performed using Life Technologies Super Script II: reagents in Table 20 and were mixed according to the manufacturer's protocol and placed on a thermocycler set to the program in Table 21. The mixture was then purified using Agencourt Ampure SPRI beads at a ratio of 1.8 μL SPRI beads per μL of sample. This sample was known as reverse transcribed sample 1.

TABLE 18

| | | | Stock | | Final | |
|---|---|---|---|---|---|---|
| Reagent | Volume | | Concentration | | Concentration | |
| RNA strand (3′ polyadenylated with open | 0.72 | ul | 556 | ng/ul | 0.2 | uM |
| reading frame SEQ ID NO: 68) | | | | | | |
| polyT hairpin (SEQ ID NO: 72 is attached at | 0.4 | ul | 50 | uM | 1 | uM |
| its 5′ end to a phosphate group and SEQ ID | | | | | | |
| NO: 72 is attached at its 3′ end to four iSpC3 | | | | | | |
| spacers which are attached at the opposite end | | | | | | |
| to the 5′ end of SEQ ID NO: 73) | | | | | | |
| T4 DNA ligase buffer | 4 | ul | 5 | x | 1 | x |
| T4 DNA ligase | 1 | ul | 2000 | U/ul | 2000 | U |
| NF H₂O | 13.88 | ul | | | | |
| Total | 20 | ul | | | | |

TABLE 19

| Number of Cycles | Step | Temp (° C.) | Time |
|---|---|---|---|
| 1 | Ligate | 16 | 2:00:00 |

TABLE 20

| | | | Stock | | Final | |
|---|---|---|---|---|---|---|
| Reagent | Volume | | Concentration | | Concentration | |
| RNA after polyT | 7 | ul | 35.5 | ng/ul | 248.5 | ng/reaction |
| hairpin ligation | | | | | | |
| dNTPs | 1 | ul | 10 uM each | | 0.5 | uM |
| NF H₂O | 5 | ul | | | | |
| First-Strand Buffer | 4 | ul | 5x | | 1x | |
| 0.1M DTT | 2 | ul | 0.1M | | 0.01M | |
| Super Script II | 1 | ul | 200 | U | 200 | U |
| Total | 20 | ul | | | | |

TABLE 21

| Number of Cycles | Step | Temp (° C.) | Time |
|---|---|---|---|
| 1 | Reverse Transcription | 42 | 0:50:00 |
| 2 | Denaturation | 70 | 0:15:00 |

Subsequently, a “non-RNA polynucleotide” (30 SpC3 spacers attached to the 5′ end of SEQ ID NO: 69 which is attached at the 3′ end to the 5′ end of four iSp18 spacers which were attached at the 3′ end to the 5′ end of SEQ ID NO: 70 which was attached at the 3′ end to the 5′ end of four 5-nitroindoles which were attached at the 3′ end to the RNA sequence CAAGGG) was ligated to the RNA polynucleotide (which was reverse transcribed in the previous step) by mixing the reagents listed in a Table 22 and placing the mixture on a thermocycler set to the program in Table 23. The mixture was then purified using Agencourt Ampure SPRI beads at a ratio of 1.8 μL SPRI beads per μL of sample. This sample was known as ligated sample 1.

TABLE 22

| | | | Stock | | Final | |
|---|---|---|---|---|---|---|
| Reagent | Volume | | Concentration | | Concentration | |
| transcribed sample 1 | 1.5 | ul | 166 | ng/ul | 250 | ng/reaction |
| T4 RNA ligase 1 | 2 | ul | 10x | | 1x | |
| reaction buffer | | | | | | |
| “non-RNA | 2.4 | ul | 50 | uM | 8.33 | uM |
| polynucleotide (see | | | | | | |
| description above) | | | | | | |
| ATP | 0.4 | ul | 50 | mM | 1 | mM |
| NF H₂O | 0.8 | ul | | | | |
| T4 RNA ligase 1 | 2.9 | ul | 10 | U/ul | 29 | U |
| PEG 8k | 10 | ul | 50% | | 25% | |
| Total | 20 | ul | | | | |

TABLE 23

| Number of Cycles | Step | Temp (° C.) | Time |
|---|---|---|---|
| 1 | Ligation | 16 | 4:00:00 |

The reagents listed in Table 24 below were mixed and incubated at 65° C. and then cooled to 4° C. at a rate of 0.1° C. per second. This sample was known as DNA/RNA construct Y.

TABLE 24

| | | | Concentration | | Final | |
|---|---|---|---|---|---|---|
| Reagent | Volume | | of Stock | | Concentration | |
| Ligated sample 1 | 9 | μl | ~1 | μM | 942 | μM |
| Anchor (SEQ ID NO: 71 attached at its 3′ | 0.36 | μl | 100 | μM | 3.77 | μM |
| end to the 5′ end of six iSp18 spacers, two | | | | | | |
| thymines and a 3′ cholesterol TEG) | | | | | | |
| 10 mM TRIS pH 7.5 50 mM NaCl | 0.19 | μl | 50 | x | 1 | x |
| Total | 9.55 | μl | | | | |

### Electrophysiology

DNA/RNA construct Y was incubated with 2 μl of 17.4 μM T4 Dda (E94C/F98W/C109A/C136A/K194L/A360C (SEQ ID NO: 8 with mutations E94C/F98W/C109A/C136A/K194L/A360C and then (ΔM1)G1)) for 20 minutes. 2.1 μl of 800 μM TMAD was then added to the incubated mixture and kept at room temperature for 10 min. This sample was then diluted into buffer (276 μL of 500 mM KCl, 25 mM potassium phosphate pH 8.0) MgCl2 (4 μL, 150 mM) and ATP (4 μL, 150 mM) giving a total volume of 300 μL.

Electrical measurements were acquired from single MspA nanopores inserted in block co-polymer in buffer (25 mM K Phosphate buffer, 150 mM Potassium Ferrocyanide, 150 mM Potassium Ferricyanide˜pH 8.0). After achieving a single pore inserted in the block co-polymer, then buffer (2 mL, 25 mM K Phosphate buffer, 150 mM Potassium Ferrocyanide, 150 mM Potassium Ferricyanide, pH 8.0) was flowed through the system to remove any excess MspA nanopores.

An excess of buffer (500 mM KCl, 25 mM potassium phosphate pH 8.0) was flowed through the system prior to the addition of DNA/RNA Construct Y and helicase. Finally, (T4 Dda (E94C/F98W/C109A/C136A/K194L/A360C (SEQ ID NO: 8 with mutations E94C/F98W/C109A/C136A/K194L/A360C and then (ΔM1)G1)), bound to DNA/RNA construct Y) was then added to the nanopore system, the experiment was run at −140 mV and helicase-controlled DNA movement monitored.

### Results:

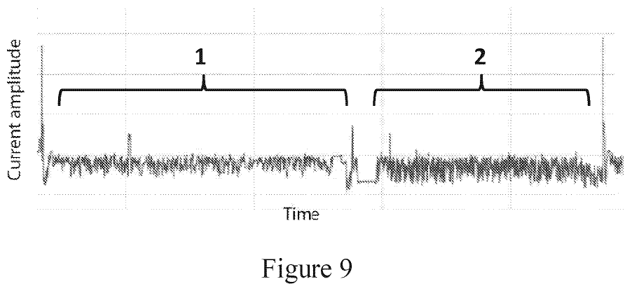

This example shows how a non-RNA polynucleotide was attached to RNA (which had been transcribed) to facilitate loading of a DNA helicase, T4 Dda (E94C/F98W/C109A/C136A/K194L/A360C (SEQ ID NO: 8 with mutations E94C/F98W/C109A/C136A/K194L/A360C and then (ΔM1)G1)), and subsequent helicase controlled movement of the construct was observed. An example of a T4 Dda (E94C/F98W/C109A/C136A/K194L/A360C (SEQ ID NO: 8 with mutations E94C/F98W/C109A/C136A/K194L/A360C and then (ΔM1)G1)) helicase-controlled movement is shown in FIG. 9.

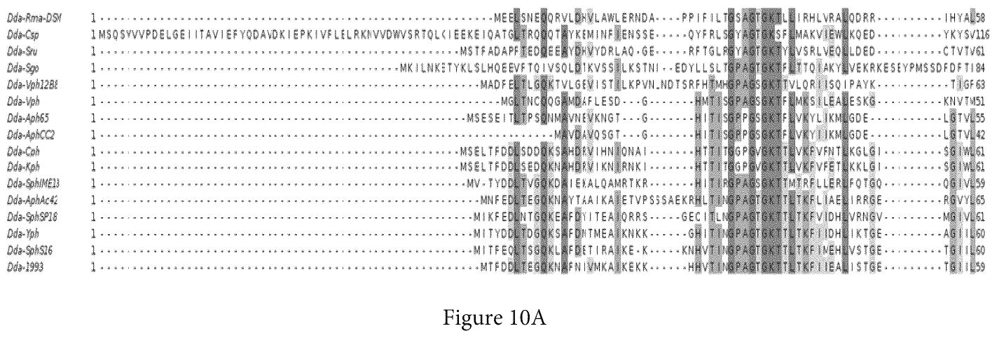

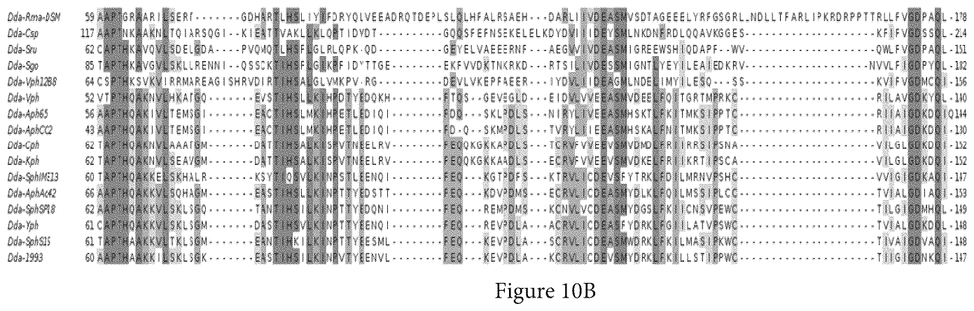

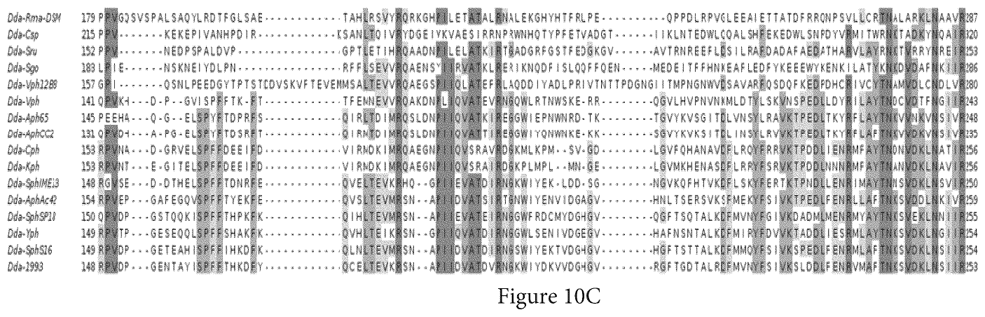

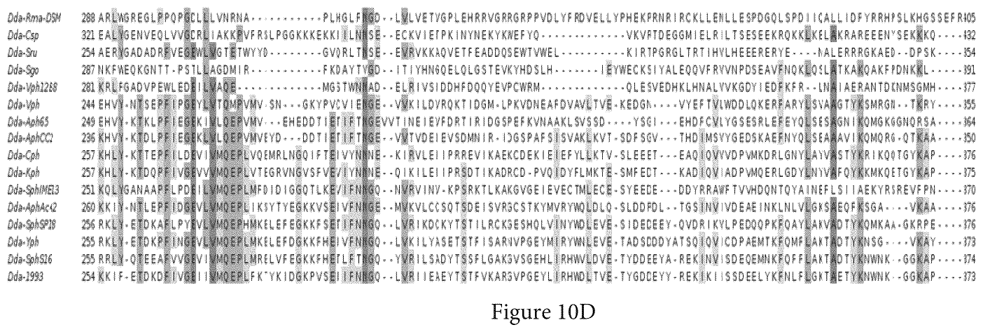

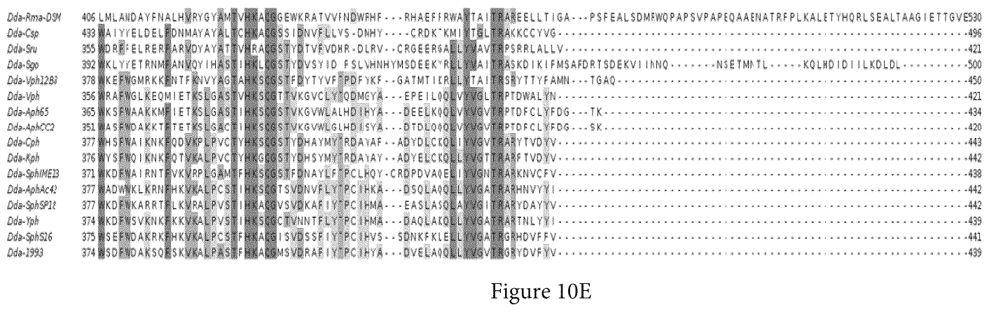

An alignment of the preferred Dda helicases of the invention (SEQ ID NOs: 8 to 23) is shown in FIGS. 10A-10E.

1. A DNA-dependent ATPase (Dda) helicase in which:
(a) at least one amino acid which interacts with one or more nucleotides in single stranded DNA (ssDNA) is substituted; and
(b) the part of the helicase which interacts with a transmembrane pore comprises one or more modifications,
wherein the helicase has the ability to control the movement of a polynucleotide.

2-24. (canceled)

25. A construct comprising a helicase according to claim 1 and an additional polynucleotide binding moiety, wherein the helicase is attached to the polynucleotide binding moiety and the construct has the ability to control the movement of a polynucleotide.

26. (canceled)

27. A polynucleotide which comprises a sequence which encodes a helicase according to claim 1.

28. A vector which comprises a polynucleotide according to claim 27 operably linked to a promoter.

29. A host cell comprising a vector according to claim 28.

30. A method of making a helicase according to claim 1 or a construct according to claim 25, which encodes a helicase according to claim 1, transfecting a cell with a vector comprising said polynucleotide operably linked to a promoter or culturing a host cell comprising said vector.

31. A method of controlling the movement of a polynucleotide, comprising contacting the polynucleotide with a DNA-dependent ATPase (Dda) helicase, in which
(a) at least one amino acid which interacts with one or more nucleotides in single stranded DNA (ssDNA) is substituted; and
(b) the part of the helicase which interacts with a transmembrane pore comprises one or more modifications,
wherein the helicase has the ability to control the movement of a polynucleotide and thereby controlling the movement of the polynucleotide.

32. A method according to claim 31, wherein the method is for controlling the movement of a polynucleotide through a transmembrane pore.

33. A method of characterising a target polynucleotide, comprising:
(a) contacting the target polynucleotide with a transmembrane pore and a helicase according to claim 1 such that the helicase or construct controls the movement of the target polynucleotide through the pore; and
(b) taking one or more measurements as the polynucleotide moves with respect to the pore wherein the measurements are indicative of one or more characteristics of the target polynucleotide and thereby characterising the target polynucleotide.

34-43. (canceled)

44. A method of forming a sensor for characterising a target polynucleotide, comprising forming a complex between (a) a pore and (b) a helicase according to claim 1 and thereby forming a sensor for characterising the target polynucleotide.

45. A method according to claim 44, wherein the complex is formed by (a) contacting the pore and the helicase or construct in the presence of the target polynucleotide and (a) applying a potential across the pore.

46. A method according to claim 45, wherein the potential is a voltage potential or a chemical potential.

47. A method according to claim 46, wherein the complex is formed by covalently attaching the pore to the helicase or construct.

48. A sensor for characterising a target polynucleotide, comprising a complex between (a) a pore and (b) a helicase according to claim 1.

49. Use of a helicase according to claim 1 to control the movement of a target polynucleotide through a pore.

50-51. (canceled)

52. A method of producing a DNA-dependent ATPase (Dda) helicase, comprising:
(a) providing a helicase; and
(b) modifying the helicase to produce a helicase in which:
(i) at least one amino acid which interacts with one or more nucleotides in single stranded DNA (ssDNA) is substituted; and
(ii) the part of the helicase which interacts with a transmembrane pore comprises one or more modifications,
wherein the helicase has the ability to control the movement of a polynucleotide.

53. A method according to claim 52, wherein the method further comprises (c) determining whether or not the resulting helicase is capable of controlling the movement of a polynucleotide.

54. A method of producing a construct according to claim 25, comprising attaching the helicase according to an additional polynucleotide binding moiety and thereby producing the construct.

55. A method according to claim 54, wherein the method further comprises determining whether or not the resulting construct is capable of controlling the movement of a polynucleotide.

56. A series of two or more helicases attached to a polynucleotide, wherein at least one of the two or more helicases is a helicase according to claim 1.
